(19) United States
(12) Patent Application Publication
(10) Pub. No.: US 2023/0330244 A1
(43) Pub. Date: Oct. 19, 2023

(54) NICOTINAMIDE PHOSPHORIBOSYLTRANSFERASE (NAMPT) INHIBITOR-CONJUGATES AND USES THEREOF

(71) Applicant: Ontario Institute for Cancer Research (OICR), Toronto (CA)

(72) Inventor: Ahmed Mamai, Mississauga (CA)

(21) Appl. No.: 18/014,890

(22) PCT Filed: Jul. 12, 2021
(86) PCT No.: PCT/CA2021/050952

§ 371 Date: Jan. 6, 2023

Related U.S. Application Data
(60) Provisional application No. 63/051,053, filed on Jul. 13, 2020.

(51) Int. Cl. A61K 47/68; C07D 401/12; C07D 213/55

(52) U.S. Cl. CPC A61K 47/6803; A61K 47/6849; C07D 401/12; C07D 213/55; A61K 47/6851; C07B 2200/05

(57) ABSTRACT

The present application relates to nicotinamide phosphoribosyltransferase (NAMPT) inhibitor-linker conjugates of Formula (I) comprising NAMPT inhibitors linked to linker groups, to processes and intermediates for their preparation, and to compositions comprising these compounds, as well as their use, for example, in the treatment or diagnosis of diseases and conditions, including, but not limited to, cancer. (I)

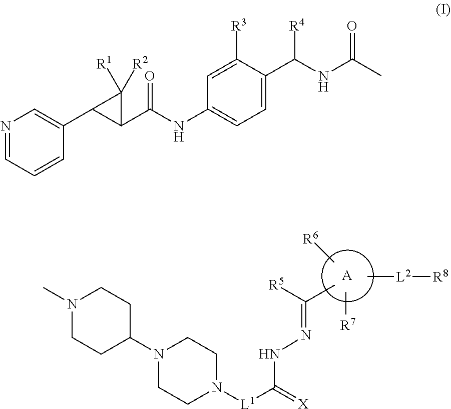

### CROSS-REFERENCE TO RELATED APPLICATIONS

The present application claims the benefit of priority from co-pending U.S. patent application No. 63/051,053, filed Jul. 13, 2020, the contents of which are incorporated herein by reference in their entirety.

### FIELD

The present application relates to nicotinamide phosphoribosyltransferase (NAMPT) inhibitor-linker conjugates comprising NAMPT inhibitors linked to linker groups, to processes and intermediates for their preparation, and to compositions comprising these compounds, as well as their use, for example, in the treatment or diagnosis of diseases and conditions, including, but not limited to, cancer. The present application also relates to deuterated 2-(pyridin-3-yl)cyclopropane-1-carboxamide derivatives as NAMPT inhibitors, to processes for their preparation, and to compositions comprising them.

### BACKGROUND

The first line therapy for many cancers is chemotherapy which targets rapidly dividing cancer cells. This modality constitutes one of the major advances in the fight against several malignancies and continues to save many human lives. However, this approach is limited by the fact that it also affects healthy cells, typically resulting in moderate to severe side effects.¹⁻² The advent of targeted therapies is starting to shift this paradigm by selectively targeting cancerous cells while not harming healthy cells hence leading to a safer class of cancer therapeutics.³⁻⁷ Biologics such as monoclonal antibodies have emerged as options for cancer therapy due to their inherent specificity for cancer associated targets and their potential to have fewer off-target effects.⁸⁻¹⁰ In addition to carrying out the immune modulating functions of antibodies,¹¹ monoclonal antibodies have been used as a means of delivering cytotoxic drugs to cancer cells with high specificity, giving way to a type of therapeutic known as antibody-drug conjugates (ADCs).¹²⁻¹⁶ ADCs have garnered considerable interest in drug discovery since they constitute a means of targeted delivery of cytotoxic agents to cancer cells. ADCs could be described as a three component entity: a cytotoxic payload, a linker and the targeting antibody. The ADC is then built by chemically attaching the cytotoxic warhead to the antibody through the linker moiety. The ADC mode of action consists of the antibody part seeking and binding to the target antigen on the tumour cell surface. Upon internalization, the drug is released inside the cell and exerts its desired cytotoxic effects. The idea of using an antibody as a vehicle to deliver selectively highly cytotoxic payloads has a huge potential. However, its application is limited by the variable in vivo stability of the linker, which will lead to lower efficacy and higher off-target effects.

ADCs (FIG. 1) contain three distinct entities: (1) an antibody designed to target a tumour-associated antigen,¹⁷⁻¹⁸(2) cytotoxic drugs,¹⁹⁻²¹ and (3) linkers that connect the drugs to the antibody.²²⁻²³ It is desirable that the ADC be stable, but upon antibody binding to the target cell and internalization, the drug is ideally released from the antibody to exert its actions.¹⁶ The efficacy and toxicity of ADCs depend heavily on the linker between the drug and the antibody and is affected by two factors: stability in plasma and drug to antibody ratio (DAR) and conjugation sites.²⁴ Currently, over 60 ADCs are in clinical trials, with 10 clinically approved: Adcetris™ (Brentuximab vedotin) targeting CD30 for anaplastic large cell lymphoma and Hodgkin's lymphoma approved in 2011, Kadcyla™ (Trastuzumab emtansine) was approved in 2013 for Her2⁺ metastatic breast cancer, Mylotarg™ (Gemtuzumab ozogamicin) targeting CD33 for acute myeloid leukemia, which was withdrawn from the market in 2010 due to excessive toxicity, was approved in 2017 under a different dosing regimen, Besponsa™ (Inotuzumab ozogamicin) was approved targeting CD22 for the treatment of refractory acute lymphoblastic leukemia²⁷⁻²⁸, Polivy™ (Polatuzumab vedotin) targeting CD79b was granted FDA approval for the treatment of diffuse large B-cell lymphomas in June 2019, Padcev™ (Enfortumab vedotin) targeting Nectin-4 was approved in December 2019 for the treatment of adult patients with locally advanced or metastatic urothelial cancers, Enhertu™ (fam-Trastuzumab deruxtecan) targeting Her2+ was approved in December 2019 as a treatment for unresectable or metastatic breast cancer following two or more prior anti-HER2 based regimens, Trodelvy™ (Sacituzumab govitecan), targeting Trop-2, was approved in April 2020 for the treatment of adult patients with metastatic triple-negative breast cancer who have received at least two prior therapies for metastatic disease, in August 2020 Blenrep™ (belantamab mafodotin-blmf) targeting BCMA was approved for the treatment of patients with relapsed or refractory multiple myeloma and finally in April 2021 Zynlonta™ (loncastuximab tesirine-lpyl) targeting CD19 was approved for the treatment of patients suffering from refractory diffuse large B-cell lymphoma.

There are currently two major classes of linkers used in ADCs: cleavable linkers such as acyl hydrazones,^{12,27,37-38} disulfides,^{20,39-42} peptides,^{22,43-46} and non-cleavable linkers.^{22,40-41} ADCs with acyl hydrazones as linkers are cleaved by the acidic environments of the lysosome. Disulfides and peptidic linkers are cleaved in thiol rich environments and by lysosomal peptidases but may have reduced potency, in part due to a greater difficulty of cleavage.^{37,47} Noncleavable linkers will only break apart upon proteolytic degradation of the antibody post-internalization. While this linkage is very stable, internalization is essential, which may reduce its range of targets.⁴⁸ Taken together it is clear that the structure of the linker has a great impact on the stability, efficacy and safety of ADCs. Moreover, cleavable linkers can release a neutral drug-linker vestige component which can have a bystander effect by killing neighboring cells that do not have the surface antigen of interest.⁴⁹ Nonclevable linkers, after proteolytic degradation, usually release a charge drug-linker vestige species that is unable to diffuse into other cells.⁵⁰

The Applicant has recently developed a platform of acyl hydrazone linkers whose lability is modulated either by steric or stereoelectronic effects and are therefore useful in the preparation of ACDs. See, for example, copending International patent application no, PCT/CA2018/051561, copending International patent application no. PCT/CA2018/051638 and copending U.S. provisional application No. 62/860,527 filed Jun. 12, 2019, entitled “Unsaturated Heterocycloalkyl and Heteroaromatic Acyl Hydrazone Linkers, Methods and Uses Thereof”.

Despite the recent successes in the ADC field with the approval of eight drugs, their payloads have in general only few modes of action: DNA damaging effect (Besponsa™ and Mylotarg™ with calicheamicin as a payload), tubulin binding mechanism (Adcertis™, Polivy™, Padcev™ and Kadcyla™ with monomethyl auristatin E and DM1 warheads), and topoisomerase I inhibition (Trodelvy™ and Enhertu™ with campthotecin derivatives as warheads). While there is a large number of ADCs undergoing clinical trials, they have payloads with only a limited diversity of mode of actions such as DNA alkylation (duocarmycins) and DNA minor groove binders (pyrrolobenzodiazepines).^{51, 52} Given the failure of several ADCs in late stage clinical trials due to severe toxicity events, there is a great need for payloads with novel mechanisms of action to, hopefully, mitigate these setbacks.

Recently, there has been extensive efforts aimed at identifying payloads with different modes of action to complement the ADC arsenal. One such approach, is to use a targeted drug that has showed promising activity in either preclinical or clinical settings but has been discontinued due to dose limiting toxicities. This repositioning as an ADC payload would deliver these potent therapeutics at a much lower dose hence expanding their therapeutic window. One such strategy is to repurpose nicotinamide phosphoribosyltransferase inhibitors (NAMPTi) as payloads. NAMPT belongs to the glycosyl transferase family. It catalyzes the conversion of nicotinamide to nicotinamide mononucleotide (NMN). It has been shown to be the rate-limiting enzyme that plays a central in role in regulating intracellular NAD+ concentration.⁵⁶ Upon NAMPT inhibition, the NAD levels decrease and can reach a critical level where normal cellular metabolism is no longer fully supported. This in turn leads to a cellular energy imbalance that can potentially cause cell death.^{57, 58}

NAMPT inhibitors have been studied as payloads using antibodies as c-Kit or HER2.⁵⁴ In addition, NAMPT inhibitors have also been used as warheads to prepare ADCs with other antibodies such as CD30.⁵⁵ Both studies produced antibody drug conjugates that showed very potent cellular activity as well as robust in vivo efficacy in different xenograft models. This constitutes strong supporting evidence for NAMPTi as viable candidates for ADC payloads.

The incorporation of deuterium in drug entities has gained momentum in the last few years. Deuterium being a hydrogen isostere has the ability to modulate the metabolic profile without having deleterious effects on the desired biological activity.⁵⁹ It is well established that the deuterium kinetic isotope effect confers to the carbon-deuterium bond a higher degree of stability than its carbon-hydrogen counterpart. Indeed, when the C—H bond breakage is the rate limiting step in a metabolic process, replacing a hydrogen with deuterium can decrease the kinetic rate of this reaction up to 10-fold which can translate into an improved stability of the compound. In many cases this leads to compound with a better pharmacokinetic profile.

The effects of deuterium substitution on metabolic stability have been reported for a very small percentage of approved drugs (see, e.g., Blake, M I et al, J Pharm Sci, 1975, 64:367-91; Foster, AB, Adv Drug Res, 1985, 14:1-40 (“Foster”); Kushner, D J et al, Can J Physiol Pharmacol, 1999, 79-88; Fisher, M B et al, Curr Opin Drug Discov Devel, 2006, 9:101-09 (“Fisher”)). In general, whether or not deuterium modification will affect a compound's metabolic properties is not predictable even when deuterium atoms are incorporated at known sites of metabolism. It is only by preparing and testing the pharmacological properties of a deuterated compound that the effect of deuteration on the rate of metabolism of the compound can be determined (see, for example, Fukuto et al. (J. Med. Chem., 1991, 34, 2871-76). One reason for this is that many compounds have multiple sites where metabolism is possible. Therefore, the site(s) where deuterium substitution is required and the extent of deuteration necessary to see an effect on metabolism, if any, will be different for each drug.

### SUMMARY

Compounds comprising 2-(pyridin-3-yl)cyclopropane-1-carboxamide based nicotinamide phosphoribosyltransferase (NAMPT) inhibitors linked to linker groups have been prepared. These NAMPT inhibitor-linker compounds are useful in antibody-drug conjugates (ADCs).

Accordingly, the present application includes a compound of Formula (I) useful in the preparation of NAMPT inhibitor-linked conjugates:

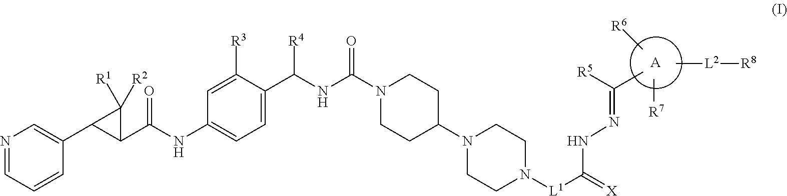

or a pharmaceutically acceptable salt and/or solvate thereof,
wherein:
Ring A is phenyl, a 5 or 6 membered unsaturated heterocycloalkyl or a 5 or 6 membered heteraromatic ring, the latter two groups comprising 1 to 4 heteroatoms selected from O, N, and S, and Ring A is optionally substituted with one or two additional substituents independently selected from CN, NO₂, halo, C₁₋₆alkyl, C₁₋₆fluoroalkyl, ═O, OR⁹ and SR⁹;
R¹ and R² are independently selected from D and H;
R³ is selected from H and halo;
R⁴ is selected from H, C₁₋₄alkyl, and C₁₋₄fluoroalkyl;
R⁵ is selected from H, C₁₋₄alkyl and C₁₋₄fluoroalkyl;
R⁶ is absent or selected from H, CN, NO₂, halo, C₁₋₆alkyl, C₁₋₆fluoroalkyl, OR¹⁰, SR¹⁰ and NR¹⁰R¹¹, and when present R⁶ is adjacent to

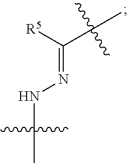

or
R⁵ and R⁶ are joined to form, together with the atoms therebetween, a 4 to 7 membered saturated or unsaturated ring, optionally containing one or two heteroatoms selected from O, N, S, S(O) and S(O)₂ and optionally substituted with one or more substituents independently selected from C₁₋₆alkyl and C₁₋₆fluoroalkyl;
R⁷ is selected from H, halo, C₁₋₆alkyl, C₁₋₆fluoroalkyl, OR¹², SR¹² and NR¹²R¹³;
R⁸ is a reactive functional group;
X is selected from O, S and NR¹⁴;
R⁹, R¹⁰, R¹¹, R¹², R¹³ and R¹⁴, are independently selected from H, C₁₋₆alkyl and C₁₋₆fluoroalkyl; and
L¹ and L² are independently a linker moiety,
provided when Ring A is phenyl, R⁵ and R⁶ are joined to form, together with the atoms therebetween, a 4 to 7 membered saturated or unsaturated ring, optionally containing one or two heteroatoms selected from O, N, S, S(O) and S(O)₂ and optionally substituted with one or more substituents independently selected from C₁₋₆alkyl and C₁₋₆fluoroalkyl, and Ring A is optionally substituted with one or two additional substituents independently selected from CN, NO₂, halo, C₁₋₆alkyl, C₁₋₆fluoroalkyl, OR⁹ and SR⁹, or
when Ring A is phenyl, R⁷ is OH and Ring A is

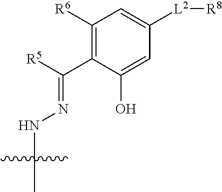

and optionally substituted with one or two additional substituents independently selected from CN, NO₂, halo, C₁₋₆alkyl, C₁₋₆fluoroalkyl, OR⁹ and SR⁹.

The present application also includes a compound of Formula (II):

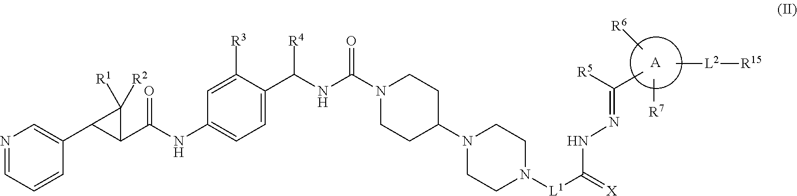

or a pharmaceutically acceptable salt and/or solvate thereof,
wherein
Ring A, L¹, L², R¹, R², R³, R⁴, R⁵, R⁶ and R⁷ are as defined above; and
R¹⁵ is a compound to be linked.

In another aspect, the present application includes an antibody-drug conjugate (ADC), the conjugate having a Formula (III):

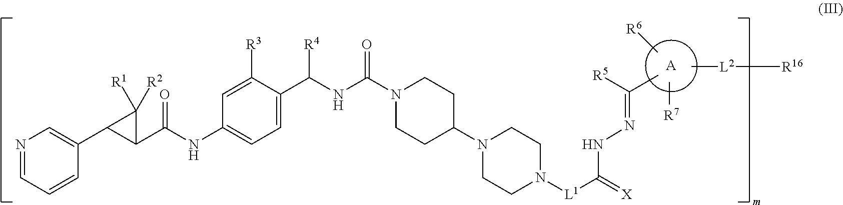

or a pharmaceutically acceptable salt and/or solvate thereof,
wherein
R¹⁶ is an antibody;
Ring A, L¹, L², R¹, R², R³, R⁴, R⁵, R⁶ and R⁷ are as defined as above; and
m is an integer from 1 to 20.

In a further aspect, the present application also includes one or more compounds of Formula (IV)

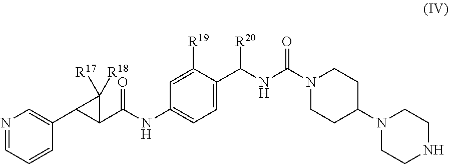

or a pharmaceutically acceptable salt and/or solvate thereof,
wherein:
R¹⁷ and R¹⁸ are independently selected from D and H;
R¹⁹ is selected from H and halo; and
R²⁰ is selected from H, C₁₋₄alkyl, and C₁₋₄fluoroalkyl;
provided at least one of R¹⁷ and R¹⁸ is D.

In another aspect, the present application includes a method of preparing an ADC of Formula (III) as defined above comprising:
(a) reacting a compound of Formula (I) as defined above with an antibody to provide the ADC of Formula (III); and optionally
(b) purifying the ADC of Formula (III).

In another aspect of the present application is a use of one or more compounds Formula (II) and/or (III), as defined above, or a pharmaceutically acceptable salt and/or solvate thereof, as a medicament and/or a diagnostic agent.

In a further aspect of the application there is provided a use of one or more compounds of Formula (II), (III), and/or (IV) as defined above, or a pharmaceutically acceptable salt and/or solvate thereof, to treat and/or diagnose cancer.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 is a schematic showing the general structure of an exemplary antibody-drug conjugate.

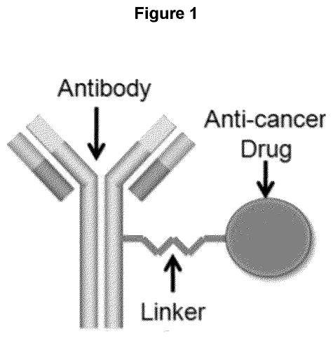

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “and/or” as used herein means that the listed items are present, or used, individually or in combination. In effect, this term means that “at least one of” or “one or more” of the listed items is used or present. The term “and/or” with respect to salts and/or solvates thereof means that the compounds of the application exist as individual salts or hydrates, as well as a combination of, for example, a salt of a solvate of a compound of the application or a solvate of a salt of a compound of the application.

As used in the present application, the singular forms “a”, “an” and “the” include plural references unless the content clearly dictates otherwise. For example, an embodiment including “a compound” should be understood to present certain aspects with one compound or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “suitable” or “suitably” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The present description refers to a number of chemical terms and abbreviations used by those skilled in the art. Nevertheless, definitions of selected terms are provided for clarity and consistency.

The term “compound(s) of the application” or “compound(s) of the present application” and the like as used herein refers to a compound of Formula (I), (II), (III) or (IV) and/or salts and/or solvates thereof.

The term “composition of the application” or “composition of the present application” and the like as used herein refers to a composition comprising one or more compounds of the application.

The compounds of the present application may further exist in varying polymorphic forms and it is contemplated that any polymorphs, or mixtures thereof, which form are included within the scope of the present application.

The compounds of the present application may further be radiolabeled and accordingly all radiolabeled versions of the compounds of the application are included within the scope of the present application. There the compounds of the application also include those in which one or more radioactive atoms are incorporated within their structure.

The term “linker moiety” as used herein refers to any molecular structure that joins two or more other molecular structures together.

The term “small molecule” as used herein refers to a molecule having a low molecular weight and with a size, for example, on the order of about 10 nm.

The term “reactive functional group” as used herein refers to a group of atoms or a single atom that will react with another group of atoms or a single atom (so called “complementary functional group”) to form a chemical interaction between the two groups or atoms.

The term “chemical interaction” as used herein refers to the formation of either a covalent or ionic bond between the reactive functional groups. The chemical interaction is one that is strong enough to append the acyl hydrazone linkers of the present application to compounds to be linked together.

The term “reacts with” as used herein generally means that there is a flow of electrons or a transfer of electrostatic charge resulting in the formation of a chemical interaction.

The term “conjugating” as used herein means to bind two molecules together via a chemical interaction.

The term “binding moiety” as used herein refers to any moiety that binds to a receptor or active site in a biological molecule. In an embodiment, the binding is specific binding, that is, the binding moiety will bind to one receptor or active site preferentially over other receptors or active sites.

The term “labelling agent” as used herein refers to any agent that is used for detection of molecules. Different types of labelling agents are known in the art depending on the form of detection to be used. For example, the labelling agent is selected from a radiolabel, a fluorescent label, a spin label, isotope label, a positron emission topography (PET) and a single-photon emission computer tomography label.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₆alkyl means an alkyl group having 1, 2, 3, 4, 5 or 6 carbon atoms. All alkyl groups are optionally fluorosubstituted unless otherwise indicated.

The term “alkylene” as used herein, whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms. All alkylene groups are optionally fluorosubstituted.

The term “alkenylene” as used herein, whether it is used alone or as part of another group, means a straight or branched chain, unsaturated alkylene group, that is, an unsaturated carbon chain that contains substituents on two of its ends and at least one double bond. The number of carbon atoms that are possible in the referenced alkenylene group are indicated by the prefix “Cₙ₁₋ₙ₂”. For example, the term C₂₋₆alkenylene means an alkenylene group having 2, 3, 4, 5 or 6 carbon atoms. All alkenylene groups are optionally fluorosubstituted, unless otherwise indicated.

The term “alkynylene” as used herein, whether it is used alone or as part of another group, means a straight or branched chain, unsaturated alkylene group, that is, an unsaturated carbon chain that contains substituents on two of its ends and at least one triple bond. The number of carbon atoms that are possible in the referenced alkynylene group are indicated by the prefix “Cₙ₁₋ₙ₂”. For example, the term C₂₋₆alkynylene means an alkynylene group having 2, 3, 4, 5 or 6 carbon atoms. All alkynylene groups are optionally fluorosubstituted, unless otherwise indicated.

The term “heterocycloalkyl” as used herein, whether it is used alone or as part of another group, refers to cyclic groups containing at least one non-aromatic ring in which one or more of the atoms are a heteroatom selected from O, S and N. Heterocycloalkyl groups are either saturated or unsaturated (i.e. contain one or more double bonds). When a heterocycloalkyl group contains the prefix “n1-n2-membered” or “n1 or n2-membered” this prefix indicates the number of atoms in the cyclic group, of which one or more are a heteroatom as defined above.

The term “unsaturated heterocycloalkyl” as used herein whether it is used alone or as part of another group, refers to cyclic groups containing at least one non-aromatic ring comprising one or more double bonds, and one or more of the atoms are a heteroatom selected from O, S and N. When a heterocycloalkyl group contains the prefix “n1-n2-membered” or “n1 or n2-membered” this prefix indicates the number of atoms in the cyclic group, of which one or more are a heteroatom as defined above.

The term “heteroaromatic” or “heteroaryl” as used herein, whether it is used alone or as part of another group, refers to cyclic groups containing at least one aromatic ring in which one or more of the atoms are a heteroatom selected from O, S and N. When a heteroaryl group contains the prefix “n1-n2-membered” or “n1 or n2-membered” this prefix indicates the number of atoms in the cyclic group, of which one or more are a heteroatom as defined above.

The term “heteroatom” as used herein, unless otherwise specified, refers to an atom other than carbon or hydrogen, and generally herein refers to O, S or N. Heteroatoms, such as N, may be substituted with additional substituents or hydrogen to fulfill valency requirements as would be known to those skilled in the art.

The term “optionally substituted” refers to groups, structures, or molecules that are either unsubstituted or are substituted with one or more substituents.

The term “fluorosubstituted” refers to the substitution of one or more, including all, hydrogens in a referenced group with fluorine.

The term “deuteroalkyl” refers to the substitution of one or more, including all, hydrogens in an alkyl group with deuterium.

The term “halo” or “halogen” as used herein, whether it is used along or as part of another group, refers to a halogen atom and includes fluoro, chloro, bromo and iodo.

The symbol “ [symbol] ” is used herein to represent the point of attachment of a group to the remainder of a molecule or chemical formula.

The term “cell” as used herein refers to a single cell or a plurality of cells and includes a cell either in a cell culture or in a subject.

The term “subject” as used herein includes all members of the animal kingdom including mammals, and suitably refers to humans. Thus the methods of the present application are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt of a compound, wherein molecules of a suitable solvent are incorporated in the crystal lattice. A suitable solvent is physiologically tolerable at the dosage administered.

The term “MS” as used herein refers to mass spectrometry.

DCM as used herein refers to dichloromethane.

DIEA or DIPEA as used herein refers to N,N-diisopropylethylamine

DMF as used herein refers to dimethylformamide.

THE as used herein refers to tetrahydrofuran.

DMSO as used herein refers to dimethylsulfoxide.

EtOAc as used herein refers to ethyl acetate.

MeOH as used herein refers to methanol.

HCl as used herein refers to hydrochloric acid.

TFA as used herein refers to trifluoroacetic acid.

NMM are used herein refers to N-methylmorpholine.

RT as used herein refers to room temperature.

RB as used herein refers to a round bottom flask.

TBAF as used herein refers to tetra-n-butylammonium fluoride.

MW as used herein refers to molecular weight.

HATU as used herein refers to 1-[bis(dimethylamino)methylene]-1H-1,2,3-triazolo[4,5-b]pyridinium 3-oxid hexafluorophosphate or hexafluorophosphate azabenzotriazole tetramethyl uronium.

HPLC as used herein refers to high performance liquid chromatography.

LCMS as used herein refers to liquid chromatography-mass spectrometry.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3^{rd} Edition, 2003, Georg Thieme Verlag (The Americas).

The term “treating” or “treatment” as used herein and as is well understood in the art, means an approach for obtaining beneficial or desired results, including clinical results. In some embodiments, beneficial or desired clinical results may include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” may also mean prolonging survival as compared to expected survival if not receiving treatment. “Treating” and “treatment” as used herein may also include prophylactic treatment. For example, a subject with early cancer may be treated to prevent progression, or alternatively a subject in remission may be treated to prevent recurrence. Treatment methods comprise administering to a subject a therapeutically effective amount of one or more of the compounds and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

As used herein, the term “effective amount” or “therapeutically effective amount” means an amount of one or more compounds that is effective, at dosages and for periods of time necessary to achieve the desired result. For example in the context of a treatment for a disease, disorder of condition, an effective amount is an amount that, for example, increases said treatment compared to the treatment without administration of the one or more compounds.

The term “administered” as used herein means administration of a therapeutically effective amount of one or more compounds or compositions to a cell, tissue, organ or subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder.

The term “cancer” as used herein refers to cellular-proliferative disease states.

The term “antibody” as used herein refers to a full-length antibody molecule or an immunologically active portion of a full-length antibody molecule, i.e., a molecule that contains an antigen binding site that immunospecifically binds antigen of a target of interest or part thereof, such targets including but not limited to, cancer cells that produce specific identifiable antigens. The term “antibody” also refers to monoclonal antibodies, polyclonal antibodies, multispecific antibodies (e.g., bispecific antibodies), and antibody fragments. Antibodies may be murine, human humanized, chimeric, or derived from other species.

The term “monoclonal antibody” as used herein refers to an antibody obtained from a population of substantially homogenous antibodies, i.e., the individual antibodies comprising the population are identical except for possible naturally occurring mutations that may be present in minor amounts. Monoclonal antibodies are highly specific, being directed towards a single antigenic site. In contrast to polyclonal antibody preparations which include different antibodies directed against different determinants (epitopes), each monoclonal antibody is directed against a single determinant on the antigen. In addition to their specificity, the monoclonal antibodies are advantageous as they can be synthesized uncontaminated by other antibodies. The modifier “monoclonal” indicates the character of the antibody as being obtained from a substantially homogenous population of antibodies, and is not to be construed as requiring production of the antibody by any particular method.

The term “ErbB” as used herein is a receptor protein tyrosine kinase which belongs to the ErbB receptor family responsible for mediating cell growth, differentiation and survival. The ErbB receptor family includes four distinct members including epidermal growth factor receptor (EGFR, ErbB1, HER1), HER2 (ErbB2 or p185^{neu}), HER3 (ErbB3) and HER4 (ErbB4 or tyro2).

The terms “epidermal growth factor receptor” or “EGFR”, includes naturally occurring and mutant forms thereof (e.g., a deletion mutant EGFR).

The term “ErbB-expressing cancer” is a cancer characterized by comprising cells which have ErbB protein present at least at their cell surface. In an embodiment, the ErbB protein is the EGFR protein which is produced at sufficient levels at the surface of the cells such that an anti-EGFR antibody can bind thereto and have a therapeutic and/or diagnostic effect with respect to the cancer.

The term “c-Kit” as used herein is a receptor protein tyrosine kinase which plays a role in cell survival, proliferation, and differentiation.

The term “c-Kit-expressing cancer” is a cancer characterized by comprising cells which have c-Kit protein present at least at their cell surface.

The term “CD30” as used herein is a cell membrane protein which belongs to the tumor necrosis factor receptor family.

The term “CD30-expressing cancer” is a cancer characterized by comprising cells which have CD30 protein present at least at their cell surface.

A “chemotherapeutic agent” or “anticancer agent” are terms that refer to a chemical compound useful in the treatment of a neoplastic disorder or cancer.

The term “drug” as used herein, is intended to refer to any compound or mixture of compounds which is capable of exerting an effective pharmacological effect.

The term DM1 as used herein refers to a compound of the formula

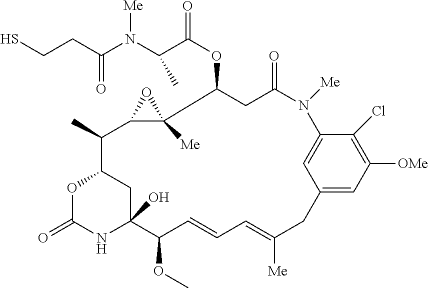 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

including pharmaceutically acceptable salts and/or solvates thereof. DM1 is also known as mertansine, and in some of its forms, emtansine.

The term “monomethyl auristatin E” or “MMAE” as used herein refers to a compound of the formula

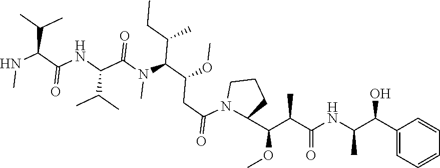 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

including pharmaceutically acceptable salts and/or solvates thereof.

The term “NAMPT” as used herein refers to the nicotinamine phosphoribosyltransferase enzyme.

The term “disease, disorder or condition” as used herein refers to a disease, disorder or condition treatable by inhibiting NAMPT.

The expression “inhibiting NAMPT” as used herein refers to inhibiting, blocking and/or disrupting NAMPT enzymatic activity in a cell. The inhibiting, blocking and/or disrupting causes a therapeutic effect in the cell.

By “inhibiting, blocking and/or disrupting” it is meant any detectable inhibition, block and/or disruption in the presence of a compound compared to otherwise the same conditions, except for in the absence in the compound.

The term “NAMPT inhibitor” as used herein refers to a compound capable of inhibiting, blocking and/or disrupting NAMPT enzymatic activity in a cell. The inhibiting, blocking and/or disrupting causes a therapeutic effect in the cell.

### II. Compounds of the Application

Compounds comprising 2-(pyridin-3-yl)cyclopropane-1-carboxamide based nicotinamide phosphoribosyltransferase (NAMPT) inhibitors linked to linker groups have been prepared. These NAMPT inhibitor-linker compounds are useful in antibody-drug conjugates (ADCs). Accordingly, these compounds are useful in in therapy, for example, in the treatment of neoplastic disorders such as cancer.

Accordingly, the present application includes a compound of Formula (I) useful in the preparation of NAMPT inhibitor-linked conjugates:

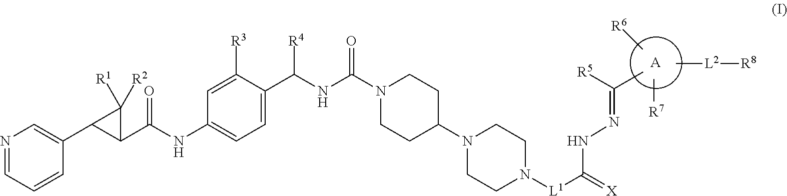

or a pharmaceutically acceptable salt and/or solvate thereof,
wherein:
Ring A is phenyl, a 5 or 6 membered unsaturated heterocycloalkyl or a 5 or 6 membered heteraromatic ring, the latter two groups comprising 1 to 4 heteroatoms selected from O, N, and S, and Ring A is optionally substituted with one or two additional substituents independently selected from CN, NO₂, halo, C₁₋₆alkyl, C₁₋₆fluoroalkyl, ═O, OR⁹ and SR⁹;
R¹ and R² are independently selected from D and H;
R³ is selected from H and halo;
R⁴ is selected from H, C₁₋₄alkyl, and C₁₋₄fluoroalkyl;
R⁵ is selected from H, C₁₋₄alkyl and C₁₋₄fluoroalkyl;
R⁶ is absent or selected from H, CN, NO₂, halo, C₁₋₆alkyl, C₁₋₆fluoroalkyl, OR¹⁰, SR¹⁰ and NR¹⁰R¹¹, and when present R⁶ is adjacent to

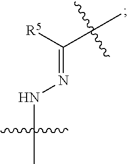

or
R⁵ and R⁶ are joined to form, together with the atoms therebetween, a 4 to 7 membered saturated or unsaturated ring, optionally containing one or two heteroatoms selected from O, N, S, S(O) and S(O)₂ and optionally substituted with one or more substituents independently selected from C₁₋₆alkyl and C₁₋₆fluoroalkyl;
R⁷ is selected from H, halo, C₁₋₆alkyl, C₁₋₆fluoroalkyl, OR¹², SR¹² and NR¹²R¹³;
R⁸ is a reactive functional group;
X is selected from O, S and NR¹⁴;
R⁹, R¹⁰, R¹¹, R¹², R¹³ and R¹⁴, are independently selected from H, C₁₋₆alkyl and C₁₋₆fluoroalkyl; and
L¹ and L² are independently a linker moiety,
provided when Ring A is phenyl, R⁵ and R⁶ are joined to form, together with the atoms therebetween, a 4 to 7 membered saturated or unsaturated ring, optionally containing one one or two heteroatoms selected from O, N, S, S(O) and S(O)₂ and optionally substituted with one or more substituents independently selected from C₁₋₆alkyl and C₁₋₆fluoroalkyl, and Ring A is optionally substituted with one or two additional substituents independently selected from CN, NO₂, halo, C₁₋₆alkyl, C₁₋₆fluoroalkyl, OR⁹ and SR⁹, or
when Ring A is phenyl, R⁷ is OH and Ring A is

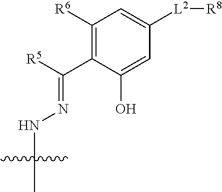

and optionally substituted with one or two additional substituents independently selected from CN, NO₂, halo, C₁₋₆alkyl, C₁₋₆fluoroalkyl, OR⁹ and SR⁹.

In some embodiments, one of R¹ and R² is D and the other is H. In some embodiments, R¹ and R² are both D. In some embodiments, R¹ and R² are both H. In some embodiments, the ring to which R¹ and R² are bonded has the following stereochemistry.

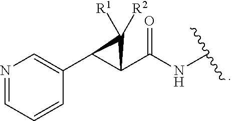

In some embodiments, R³ is selected from H and F. In some embodiments, R³ is F.

In some embodiments, R⁴ is other than H and the stereochemistry of the carbon atom to which R⁴ is attached is an S configuration. In some embodiments, R⁴ is other than H and the stereochemistry of the carbon atom to which R⁴ is attached is an R configuration. In some embodiments, R⁴ is selected from H, CH₃ and CF₃. In some embodiments, R⁴ is selected from CH₃ and CF₃. In some embodiments, R⁴ is selected from CH₃ and CF₃ and the stereochemistry of the carbon atom to which R⁴ is attached is an S configuration. In some embodiments, R⁴ is H.

In some embodiments, X is O.

In some embodiments, Ring A is a 5 or 6 membered heteroaromatic ring, and Ring A is optionally substituted with one or two additional substituents independently selected from CN, NO₂, halo, C₁₋₆alkyl, C₁₋₆fluoroalkyl, OR⁹ and SR⁹.

In some embodiments, Ring A is selected from pyridinyl, pyrimidinyl, pyridazinyl, pyrazinyl, triazinyl, tetrazinyl, thienyl, furanyl, pyrrolyl, triazolyl, thiazolyl, oxazolyl and pyrazolyl. In some embodiments, Ring A is a 6 membered heteroaromatic ring. In some embodiments, Ring A is selected from pyridinyl, pyrimidinyl, pyrazinyl and pyridazinyl. In some embodiments, L² is located in the position para to

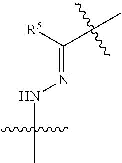

### on Ring A.

In some embodiments, when Ring A is a 5 or 6 membered heteroaromatic ring, the one or two additional substituents are independently selected from CN, NO₂, halo, C₁₋₆alkyl, C₁₋₆fluoroalkyl, OR⁹ and SR⁹. In some embodiments, the one or two additional substituents are independently selected from CN, halo, C₁₋₆alkyl and C₁₋₆fluoroalkyl. In some embodiments, the one or two additional substituents are independently selected from halo, C₁₋₆alkyl and C₁₋₆fluoroalkyl. In some embodiments, the one or two additional substituents are independently selected from CH₃, CF₃, CH₂CH₃, CH₂CH₂F, CH₂CF₂H and CH₂CF₃.

In some embodiments, when Ring A is a 5 or 6 membered heteroaromatic ring, R⁵ is selected from H, CH₃, CF₃, CH₂CH₃, CH₂CH₂F, CH₂CF₂H and CH₂CF₃. In some embodiments, R⁵ is selected from H and CH₃. In some embodiments, R⁵ is CH₃.

In some embodiments, when Ring A is a 5 or 6 membered heteroaromatic ring, R⁶ is absent. In some embodiments, when Ring A is a 5 or 6 membered heteroaromatic ring, R⁶ is selected from H, CN, NO₂, halo, C₁₋₆alkyl, C₁₋₆fluoroalkyl, OR¹⁰ and SR¹⁰. In some embodiments, R⁶ is selected from H, CN, halo, C₁₋₆alkyl and C₁₋₆fluoroalkyl. In some embodiments, R⁶ is selected from H and CH₃. In some embodiments, R⁶ is H.

In some embodiments, when Ring A is a 5 or 6 membered heteroaromatic ring, R⁵ and R⁶ are joined to form, together with the atoms therebetween, a 5 to 6 membered saturated or unsaturated carbocyclic ring, optionally substituted with one or more substituents independently selected from C₁₋₆alkyl and C₁₋₆fluoroalkyl. In some embodiments, R⁵ and R⁶ are joined to form a 6 membered saturated or unsaturated ring, optionally substituted with one or two substituents independently selected from C₁₋₆alkyl and C₁₋₆fluoroalkyl. In some embodiments, R⁵ and R⁶ are joined to form a 6 membered unsaturated ring

In some embodiments, when Ring A is a 5 or 6 membered heteroaromatic ring, R⁵ and R⁶ are joined to form, together with the atoms therebetween, a 4 to 7 membered saturated or unsaturated ring, containing one heteroatom selected from O, N, S, S(O) and S(O)₂ and optionally substituted with one or two substituents independently selected from C₁₋₆alkyl and C₁₋₆fluoroalkyl.

In some embodiments, when Ring A is a 5 or 6 membered heteroaromatic ring, R⁷ is selected from H, halo, OR¹², C₁₋₆alkyl and C₁₋₆fluoroalkyl. In some embodiments, R⁷ is selected from H, OH, CH₃, CF₃, CH₂CH₃, CH₂CH₂F, CH₂CF₂H and CH₂CF₃.

In some embodiments, Ring A is a 5 or 6 membered unsaturated heterocycloalkyl ring, and Ring A is optionally substituted with one or two additional substituents independently selected from CN, NO₂, halo, C₁₋₆alkyl, C₁₋₆fluoroalkyl, ═O, OR⁹ and SR⁹. In some embodiments, Ring A is triazolyl and the one or two additional substituents are independently selected from CN, NO₂, halo, C₁₋₆alkyl, C₁₋₆fluoroalkyl, OR⁹ and SR⁹, suitably one or two substituents independently selected from C₁₋₆alkyl and C₁₋₆fluoroalkyl, more suitably one or two substituents independently selected from CH₃, CF₃, CH₂HC₃, CH₂CH₂F, CH₂CF₂H and CH₂CF₃.

In some embodiments, Ring A is triazolonyl. In some embodiments, Ring A is triazolonyl, and the compound of Formula I has the following structure:

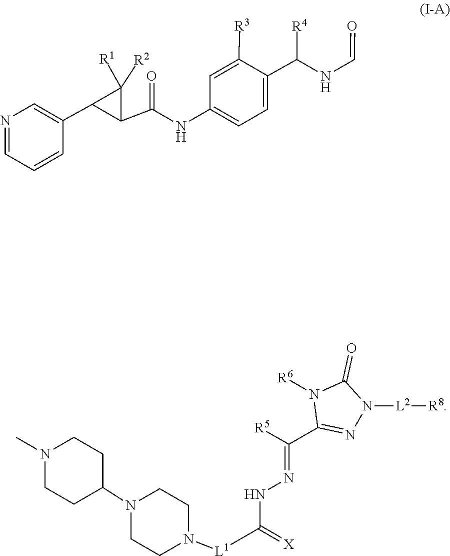

In some embodiments, Ring A is phenyl and R⁵ and R⁶ are joined to form, together with the atoms therebetween, a 5 to 7 membered unsaturated ring, containing one or two heteroatoms selected from O, N, S, S(O) and S(O)₂, and Ring A is optionally substituted with one or two additional substituents independently selected from CN, NO₂, halo, C₁₋₆alkyl, C₁₋₆fluoroalkyl, OR⁹ and SR⁹. In some embodiments, Ring A is phenyl and R⁵ and R⁶ are joined to form, together with the atoms therebetween, a 5 to 6 membered unsaturated ring, containing one heteroatom selected from O, N, S, S(O) and S(O)₂, and Ring A is optionally substituted with one or two additional substituents independently selected from CN, NO₂, halo, C₁₋₆alkyl, C₁₋₆fluoroalkyl, OR⁹ and SR⁹. In some embodiments, the one or two additional substituents are independently selected from H, CN, F and C₁₋₆alkyl. In some embodiments, the one or two additional substituents are independently selected from H, F and C₁₋₆alkyl.

In some embodiments, when Ring A is phenyl, R⁵ and R⁶ are joined to form, together with the atoms therebetween, a 5 to 6 membered unsaturated ring, containing one heteroatom selected from O, N and S. In some embodiments, the heteroatom is N. In some embodiments, the heteroatom is O.

In some embodiments, Ring A is phenyl and R⁵ and R⁶ are joined to form, together with the atoms therebetween, a 5 to 7 membered unsaturated carbocyclic ring, and Ring A is optionally substituted with one or two additional substituents independently selected from CN, NO₂, halo, C₁₋₆alkyl, C₁₋₆fluoroalkyl, OR⁹ and SR⁹. In some embodiments, Ring A is phenyl and R⁵ and R⁶ are joined to form, together with the atoms therebetween, a 5 or 6 membered unsaturated carbocyclic ring, and Ring A is optionally substituted with one or two additional substituents independently selected from CN, NO₂, halo, C₁₋₆alkyl, C₁₋₆fluoroalkyl, OR⁹ and SR⁹. In some embodiments, the one or two additional substituents are independently selected from H, CN, halo, C₁₋₆alkyl and C₁₋₆fluoroalkyl. In some embodiments, the one or two additional substituents are independently selected from H, CN, halo and C₁₋₆alkyl. In some embodiments, the one or two additional substituents are independently selected from H, halo and C₁₋₆alkyl.

In some embodiments, Ring A is

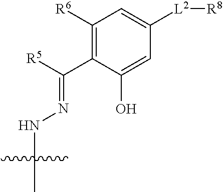

optionally substituted with one or two additional substituents independently selected from CN, NO₂, halo, C₁₋₆alkyl, C₁₋₆fluoroalkyl, OR⁹ and SR⁹.

In some embodiments, when Ring A is

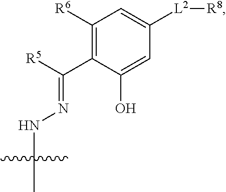

R⁵ is selected from H, CH₃, CF₃, CH₂CH₃, CH₂CH₂F, CH₂CF₂H and CH₂CF₃. In some embodiments, R⁵ is selected from H and CH₃. In some embodiments, R⁵ is CH₃.

In some embodiments, when Ring A is

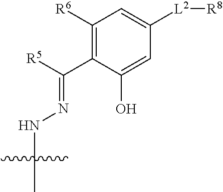

R⁶ is selected from H, CN, NO₂, halo, C₁₋₆alkyl, C₁₋₆fluoroalkyl, OR¹⁰ and SR¹⁰. In some embodiments, R⁶ is selected from H, CN, halo, C₁₋₆alkyl and C₁₋₆fluoroalkyl. In some embodiments, R⁶ is selected from H and CH₃. In some embodiments, R⁶ is H.

In some embodiments, R⁷ is located in a position ortho to

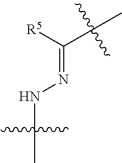

on Ring A. In some embodiments, R⁷ is selected from H, Cl, F, CH₃, CF₃ and OR¹². In some embodiments, R⁷ is OR¹².

In some embodiments, each R⁹, R¹⁰, R¹¹, R¹², R¹³ and R¹⁴ are independently selected from H, C₁₋₄alkyl and C₁₋₄fluoroalkyl. In some embodiments, each R⁹, R¹⁰, R¹¹, R¹², R¹³ and R¹⁴ are independently selected from H and C₁₋₄alkyl. In some embodiments, R₁₂ is H. In some embodiments, R¹² is selected from methyl, ethyl, propyl, isopropyl, sec-butyl, n-butyl and t-butyl. In some embodiments, R¹² and R¹³ are independently H or methyl. In some embodiments, R¹¹ and R¹⁴ are independently H. In some embodiments, R¹⁰ and R¹² are independently selected from H and CH₃.

In some embodiments, L¹ and L² independently comprise at least one ester, carbonate, carbamate or amide linkage although a person skilled in the art would appreciate that other linker moieties, such as ethers, sulfones, sulfoxides, thioethers, thioamides, thioesters and/or amines can additionally, or alternatively, be present. In some embodiments, L¹ and L² independently also comprise one or more C₁-C₂₀alkylene groups, C₂-C₂₀alkenylene groups or C₂-C₂₀alkynylene groups.

In some embodiments, L¹ and L² are independently selected from a direct bond, Z, R^{a}, Z—R^{a}, R^{a}—Z, R^{a}—Z—R^{b} and Z—R^{a}—Z^{a}, wherein Z and Z^{a} are independently selected from O, S, S(O), SO₂, NH, N(C₁₋₆alkyl), C(Q), C(Q)Y, YC(Q), YC(Q)Y^{a}, (C₁₋₆alkyleneY)ₚ and Y—(C₁₋₆alkyleneY)ₚ, wherein R^{a} and R^{b} are independently selected from C₁₋₁₀alkylene, C₂₋₁₀alkenylene and C₂₋₁₀alkynylene; Q, Y and Y^{a} are independently selected from O, S, NH and N(C₁₋₆alkyl); and p is selected from 1, 2, 3, 4, 5 and 6.

In some embodiments, R^{a} and R^{b} are independently selected from C₁₋₆alkylene, C₂₋₆alkenylene and C₂₋₆alkynylene. In some embodiments, R^{a} and R^{b} are independently selected from C₁₋₆alkylene.

In some embodiments, Q, Y and Y^{a} are independently selected from O, S, NH and N(CH₃).

In some embodiments Z and Z^{a} are independently selected from O, S, S(O), SO₂, NH, N(CH₃), C(O), C(O)NH, NHC(O), NHC(O)O, OC(O)O, NHC(O)NH, OC(O)NH, NHC(NH)NH, (C₁₋₆alkyleneO)ₚ and O—(C₁₋₆alkyleneO)ₚ. In some embodiments, Z and Z^{a} are independently selected from O, NH, C(O)NH and NHC(O).

In some embodiments, L¹ is selected from C₁₋₁₀alkyleneS and C₁₋₁₀alkylene.

In some embodiments L² is selected from OC(O)C₁₋₁₀alkyleneO, NHC(O)C₁₋₁₀alkyleneO, C₁₋₆alkyleneO, OC(O)C₁₋₁₀alkyleneNH, NHC(O)C₁₋₁₀alkyleneNH, C₁₋₆alkyleneNH, C(O)C₁₋₁₀alkyleneO and C(O)C₁₋₁₀alkyleneNH. In some embodiments L² is selected from OC(O)C₁₋₁₀alkyleneO, NHC(O)C₁₋₁₀alkyleneO, C₁₋₆alkyleneO, OC(O)C₁₋₁₀alkyleneNH, NHC(O)C₁₋₁₀alkyleneNH, C₁₋₆alkyleneNH, C(O)C₁₋₁₀alkyleneO, C(O)C₁₋₁₀alkyleneNH, NHC(O)C₁₋₁₀alkyleneC(O)NH and NHC₁₋₁₀alkyleneC(O)NH. In some embodiments, L² is selected from C₁₋₁₀alkyleneC(O)NH, C₁₋₁₀alkyleneO, C₁₋₁₀alkyleneC(O)NH and C₁₋₁₀alkyleneO.

In some embodiments, L² is located in a position para to

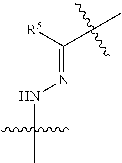

### on Ring A.

In some embodiments, the reactive functional group R⁸ is nucleophilic and is reactive to a complementary electrophilic group present on a compound to be attached. Useful electrophilic groups on the compound include, but are not limited to, aldehyde, olefin, acetylene, carboxylic acid, ester and ketone functional groups. In some embodiments, the reactive functional group R⁸ is electrophilic and is reactive to a complementary nucleophilic group present on the compound to be attached. Useful nucleophilic groups on the compound include, but are not limited to, hydrazide, oxime, amino, thiol, hydrazine, thiosemicarbazone, hydrazine carboxylate and aryl hydrazide. In some embodiments, the nucleophilic group is selected from amino and thiol groups provided by reactive lysine and cysteine amino acid groups, respectively.

In some embodiments, the nucleophilic and electrophilic reactive functional group R⁸ includes, but is not limited to, Michael addition acceptors, olefins, acetylenes, alcohols, phenols, ethers, oxides, halides, aldehydes, ketones, carboxylic acids, esters, amines, thiols, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids, isonitriles, amidines, imides, imidates, nitrones, hydroxylamines, oximes, hydroxamic acids, thiohydroxamic acids, allenes, ortho esters, N-hydroxysuccinimide esters, maleimide, sulfites, enamines, ureas, semicarbazides, carbodiimides, carbamates, imines, azides, azo compounds or nitroso compounds.

In some embodiments, the reactive functional group R⁸ is selected from a nucleophilic group and an electrophilic group. In some embodiments, the reactive functional group R⁸ is selected from Michael addition acceptors, N-hydroxysuccinimide esters, amines, maleimide and thiols.

In some embodiments, the compound of Formula (I) has the following structure:

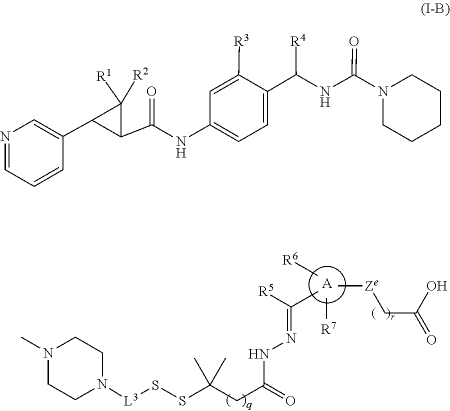

or a pharmaceutically acceptable salt and/or solvate thereof,
wherein
Ring A, R¹, R², R³, R⁴, R⁵, R⁶ and R⁷ are as defined above;
Z^{e} is C(O)NH or O;
L³ is a linker moiety;
q is 1, 2, 3, 4, 5, 6, 7 or 8; and
r is 1, 2, 3, 4, 5, 6, 7 or 8.

In some embodiments, the compound of Formula (I) has the following structure:

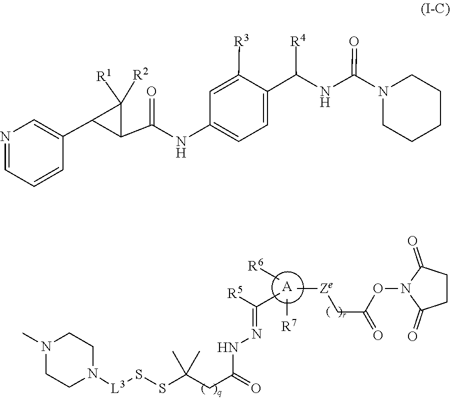

or a pharmaceutically acceptable salt and/or solvate thereof,
wherein
Ring A, R¹, R², R³, R⁴, R⁵, R⁶ and R⁷ are as defined above;
Z^{e} is C(O)NH or O;
L³ is a linker moiety;
q is 1, 2, 3, 4, 5, 6, 7 or 8; and
r is 1, 2, 3, 4, 5, 6, 7 or 8.

In some embodiments, q in the compounds of Formula (I-B) and (I-C) is 2, 3 or 4. In some embodiments, q is 1 or 2. In some embodiments, q is 1. In some embodiments, r in the compounds of Formula (I-B) and (I-C) is 2, 3 or 4. In some embodiments, r is 3.

In some embodiments L³ in the compounds of Formula (I-B) and (I-C) is selected from a direct bond, Z^{b}, R^{c}, Z^{b}—R^{c}, R^{c}—Z^{b}, R^{c}—Z^{b}—R^{d} and Z^{b}—R^{c}—Z^{c}, wherein Z^{b} and Z^{c} are independently selected from O, S, S(O), SO₂, NH, N(C₁₋₆alkyl), C(Q^{a}), C(Q^{a})Y^{b}, Y^{b}C(Q^{a}), Y^{b}C(Q^{a})Y^{c}, (C₁₋₆alkyleneY^{b})ₚ and Y^{b}—(C₁₋₆alkyleneY^{b})ₚ, wherein R^{c} and R^{d} are independently selected from C₁₋₁₀alkylene, C₂₋₁₀alkenylene and C₂₋₁₀alkynylene; Q^{a}, Y^{b} and Y^{c} are independently selected from O, S, NH and N(C₁₋₆alkyl); and p is selected from 1, 2, 3, 4, 5 and 6.

In some embodiments, R^{c} and R^{d} in the compounds of Formula (I-B) and (I-C) are independently selected from C₁₋₆alkylene, C₂₋₆alkenylene and C₂₋₆alkynylene. In some embodiments, R^{c} and R^{d} are independently selected from C₁₋₆alkylene.

In some embodiments, Q^{a}, Y^{b} and Y^{c} in the compounds of Formula (I-B) and (I-C) are independently selected from O, S, NH and N(CH₃).

In some embodiments Z^{b} and Z^{c} in the compounds of Formula (I-B) and (I-C) are independently selected from O, S, S(O), SO₂, NH, N(CH₃), C(O), C(O)NH, NHC(O), NHC(O)O, OC(O)O, NHC(O)NH, OC(O)NH, NHC(NH)NH, (C₁₋₆alkyleneO)ₚ and O—(C₁₋₆alkyleneO)ₚ.

In some embodiments L³ in the compounds of Formula (I-B) and (I-C) is selected from OC(O)C₁₋₁₀alkyleneO, NHC(O)C₁₋₁₀alkyleneO, C₁₋₆alkyleneO, OC(O)C₁₋₁₀alkyleneNH, NHC(O)C₁₋₁₀alkyleneNH, C₁₋₆alkyleneNH, C(O)C₁₋₁₀alkyleneO and C(O)C₁₋₁₀alkyleneNH.

In some embodiments, the compound of Formula (I) is selected from:

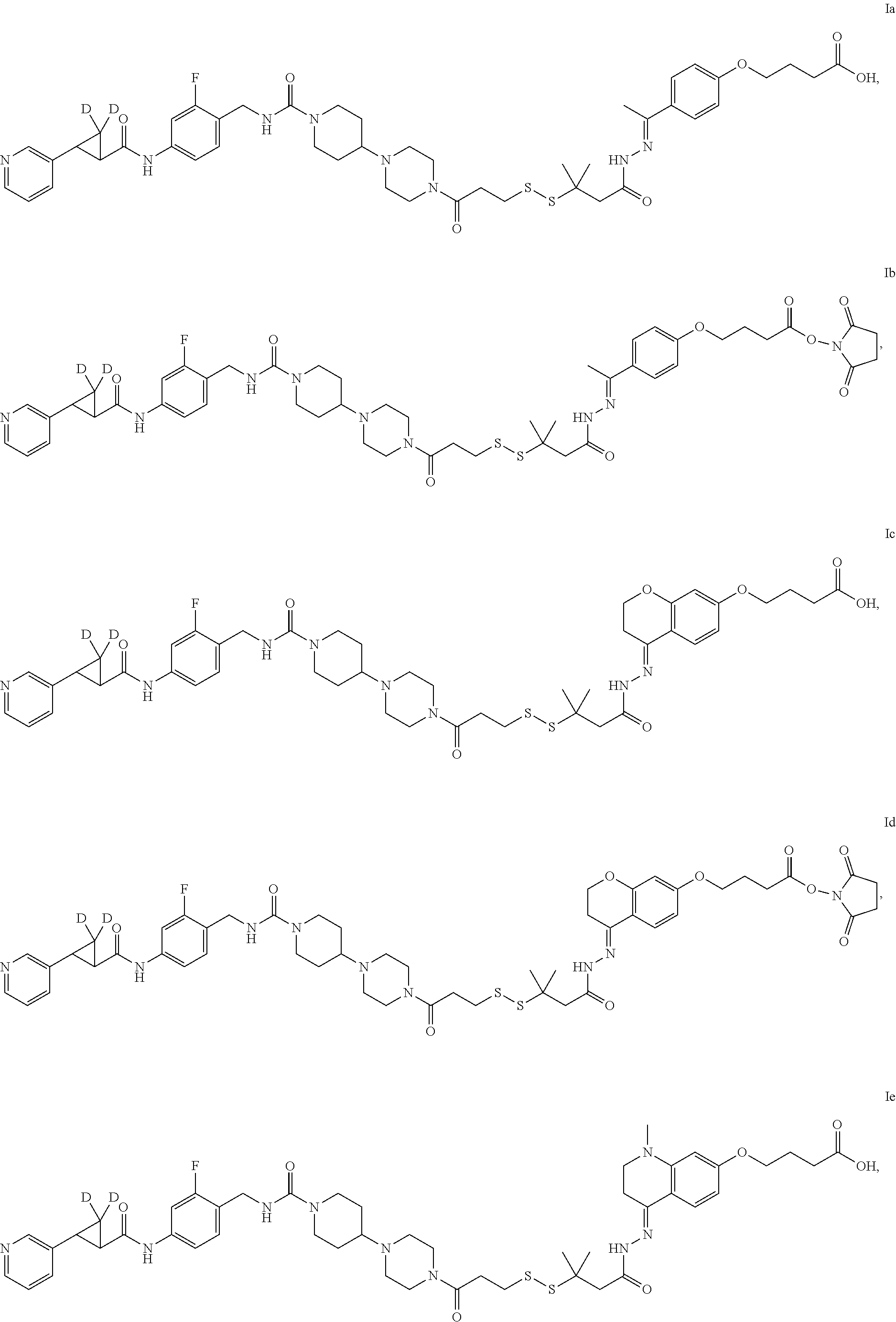

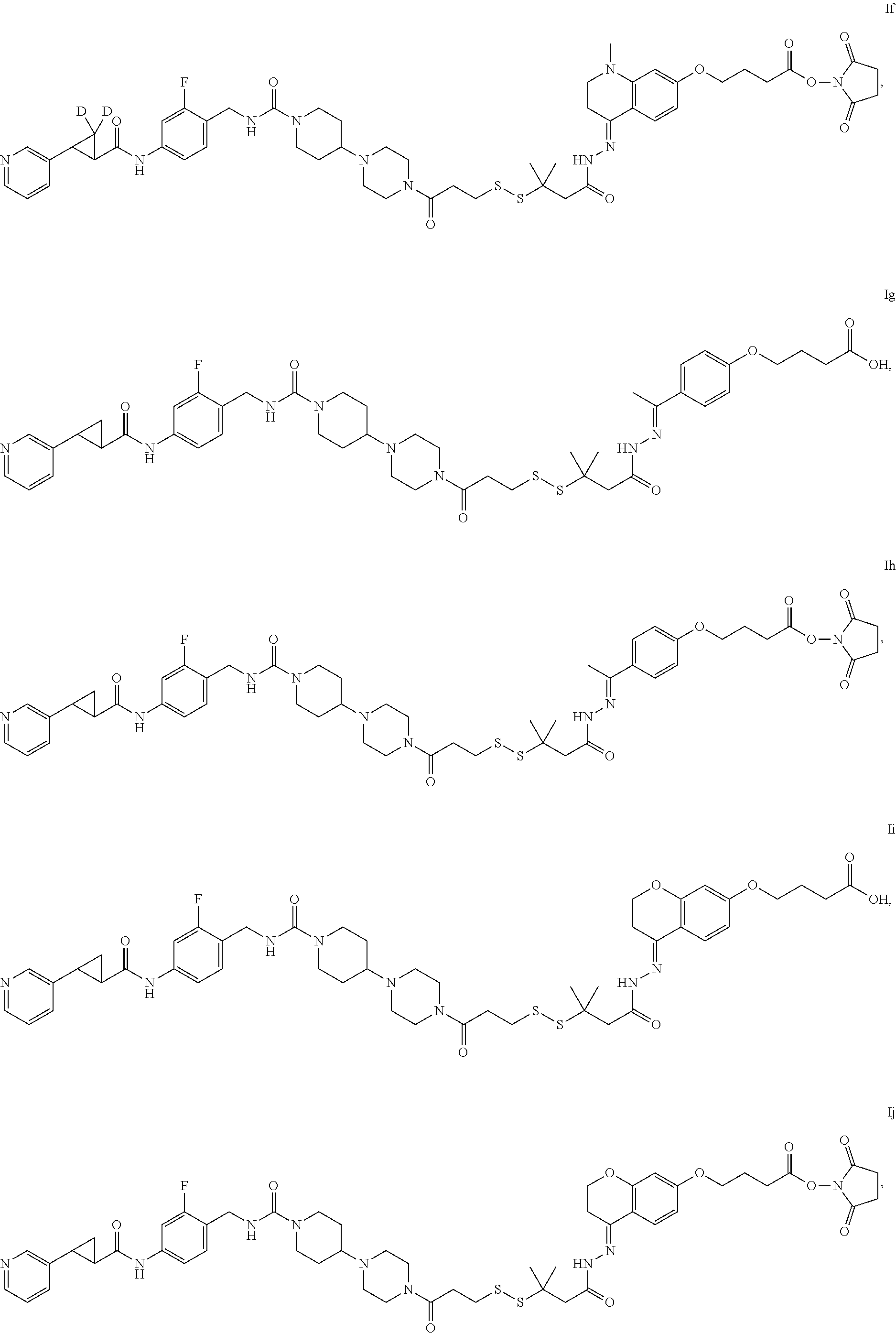

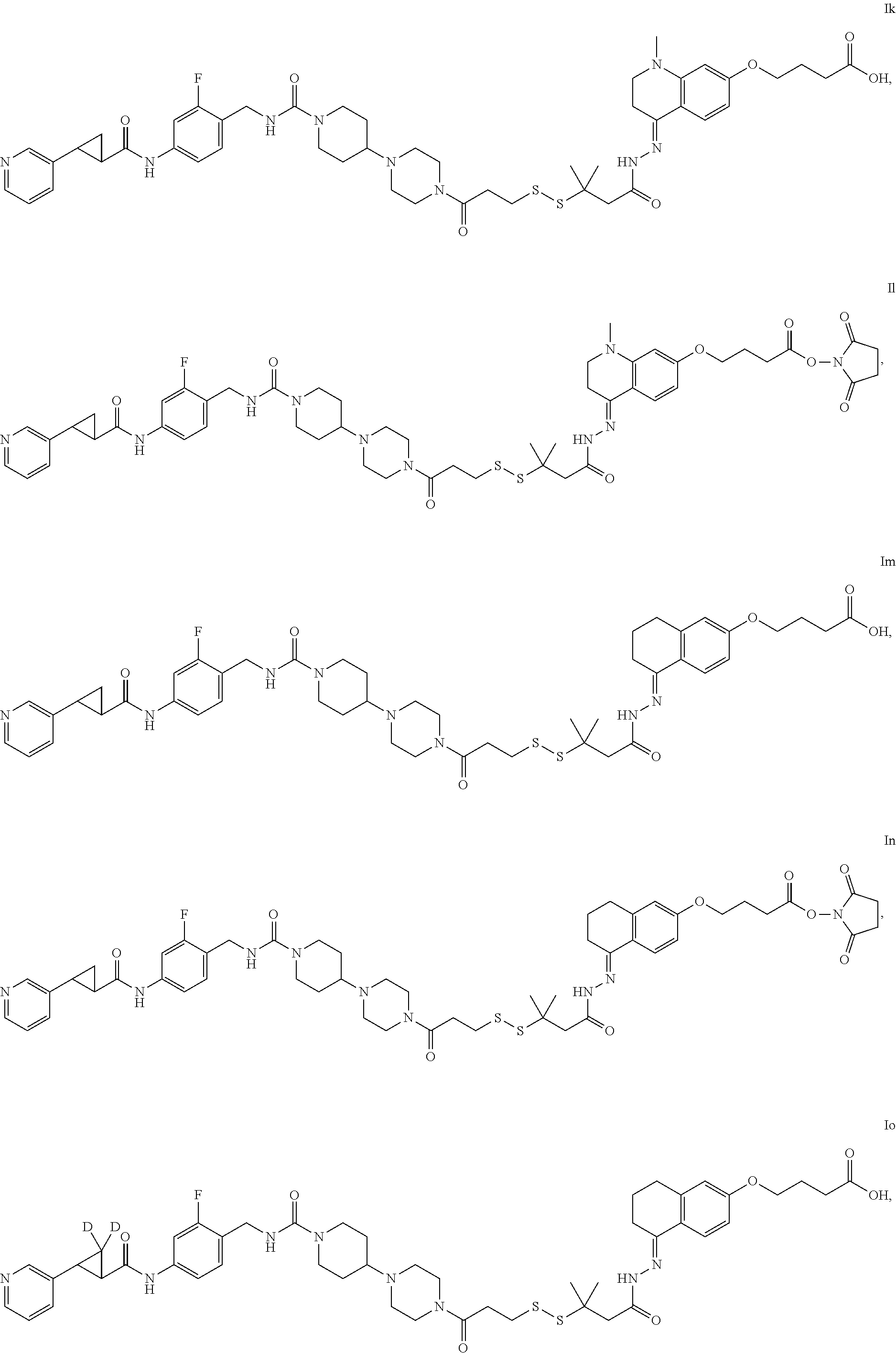

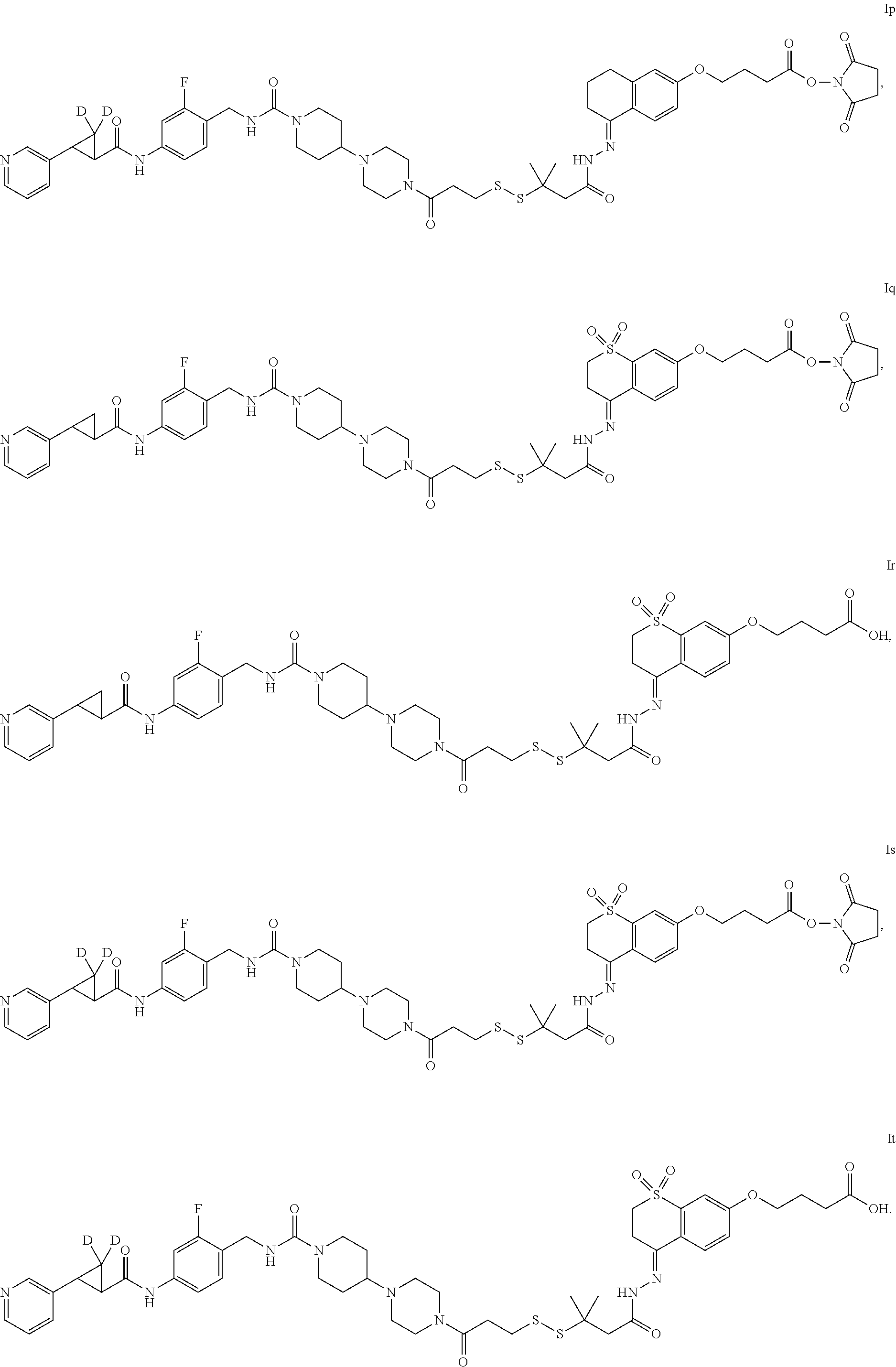

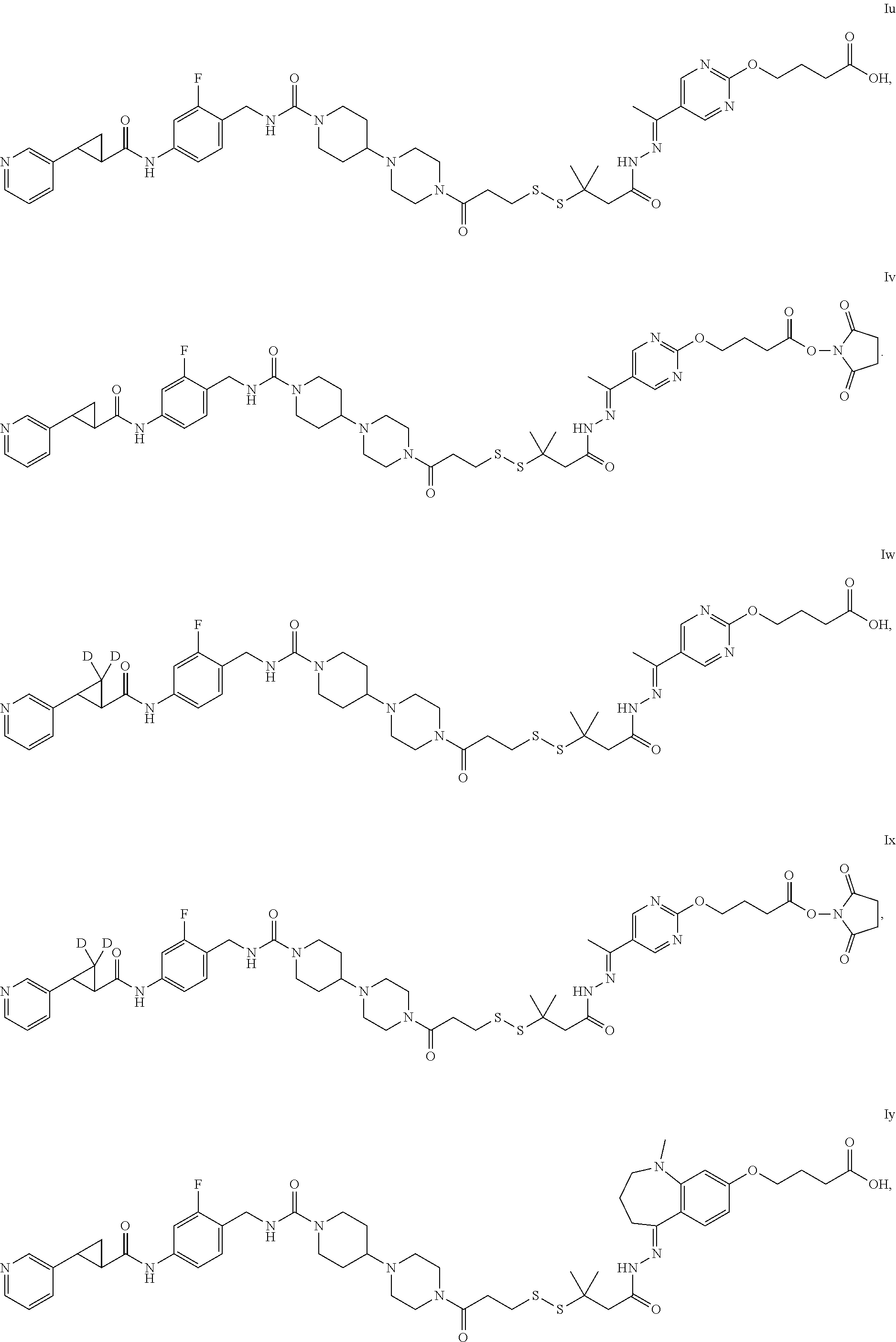

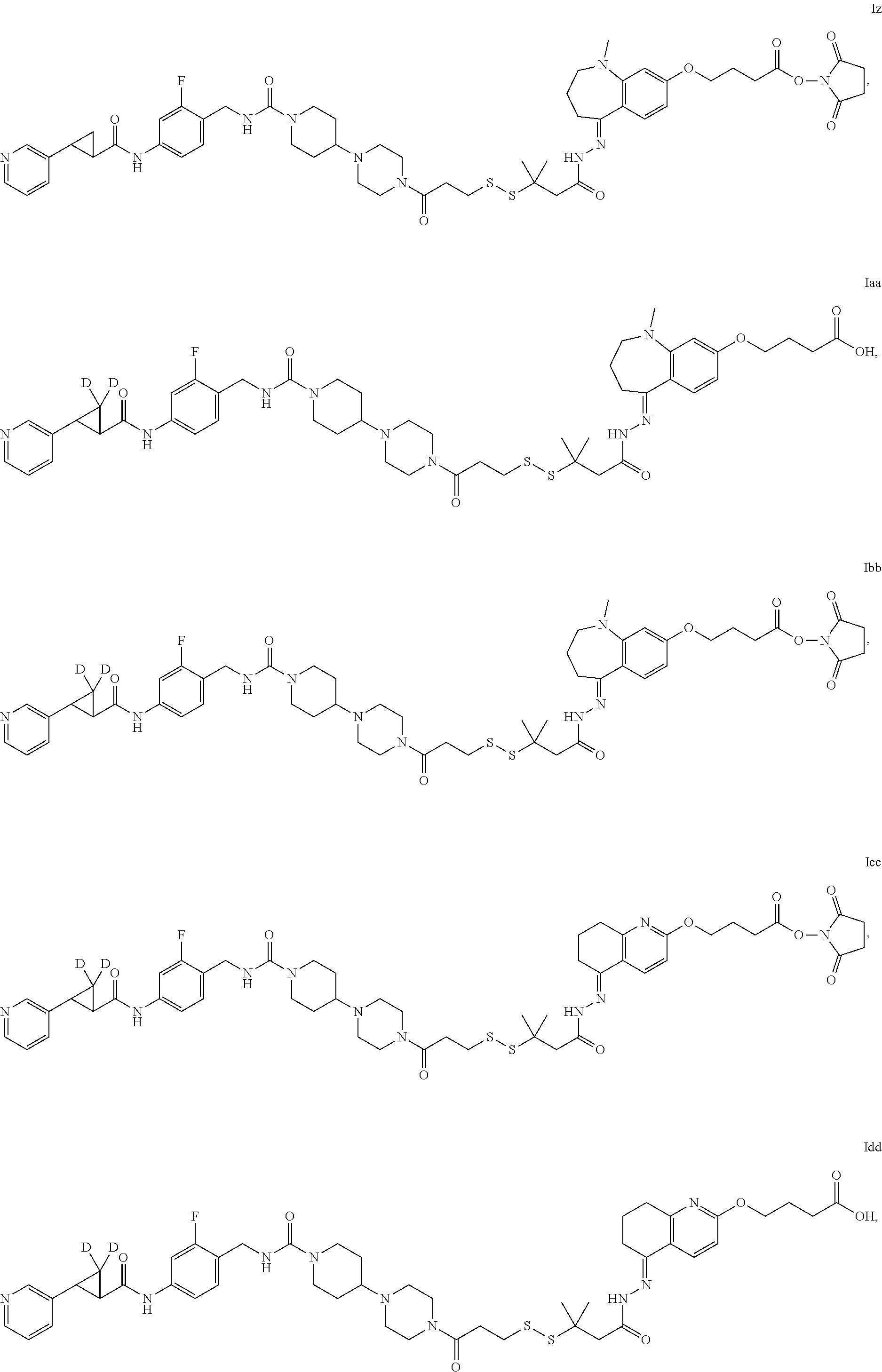

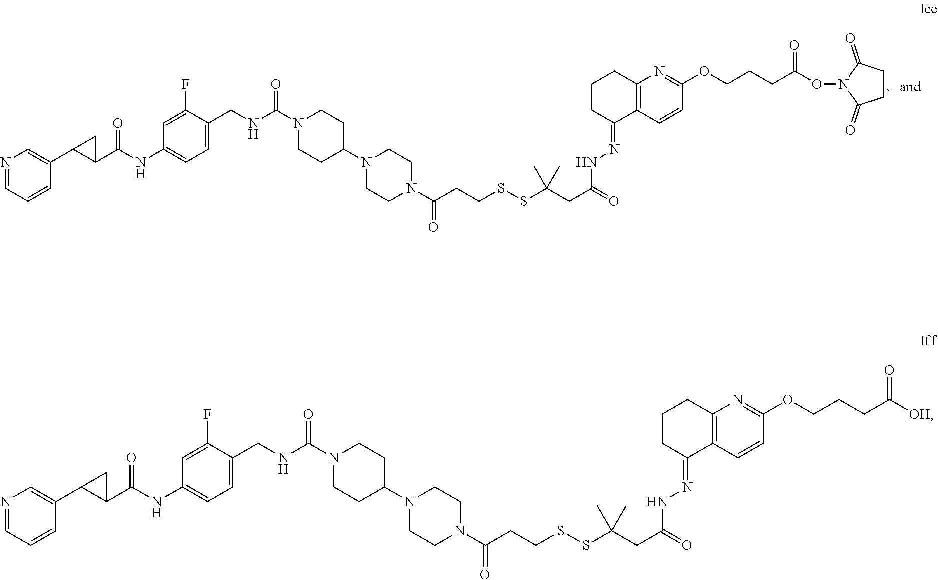 [SMILES: CC(C)(CC(=O)N/N=C1\CCCc2nc(OCCCC(=O)ON3C(=O)CCC3=O)ccc21)SSCCC(=O)N1CCN(C2CCN(C(=O)NCc3ccc(NC(=O)C4CC4c4cccnc4)cc3F)CC2)CC1 ; CC(C)(CC(=O)N/N=C1\CCCc2nc(OCCCC(=O)O)ccc21)SSCCC(=O)N1CCN(C2CCN(C(=O)NCc3ccc(NC(=O)C4CC4c4cccnc4)cc3F)CC2)CC1]

or a pharmaceutically acceptable salt and/or solvate thereof.

In some embodiments, the compound of Formula (I), or a pharmaceutically acceptable salt and/or solvate thereof, has the following structure:

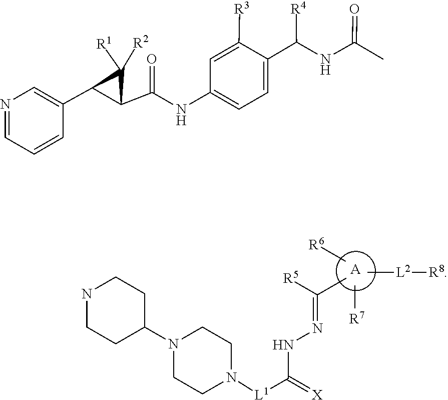

Accordingly, in some embodiments, the compound of Formula (I) is selected from:

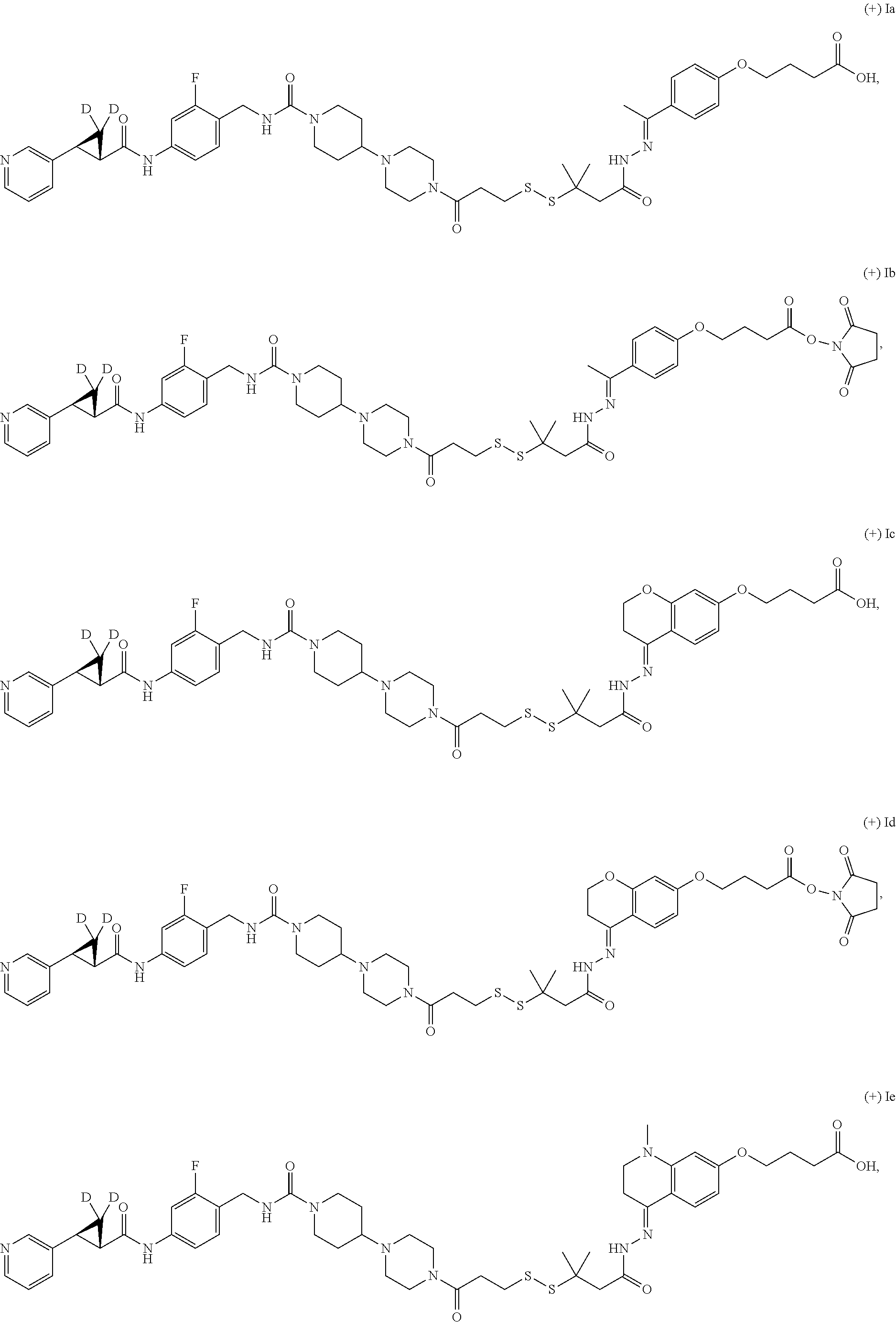

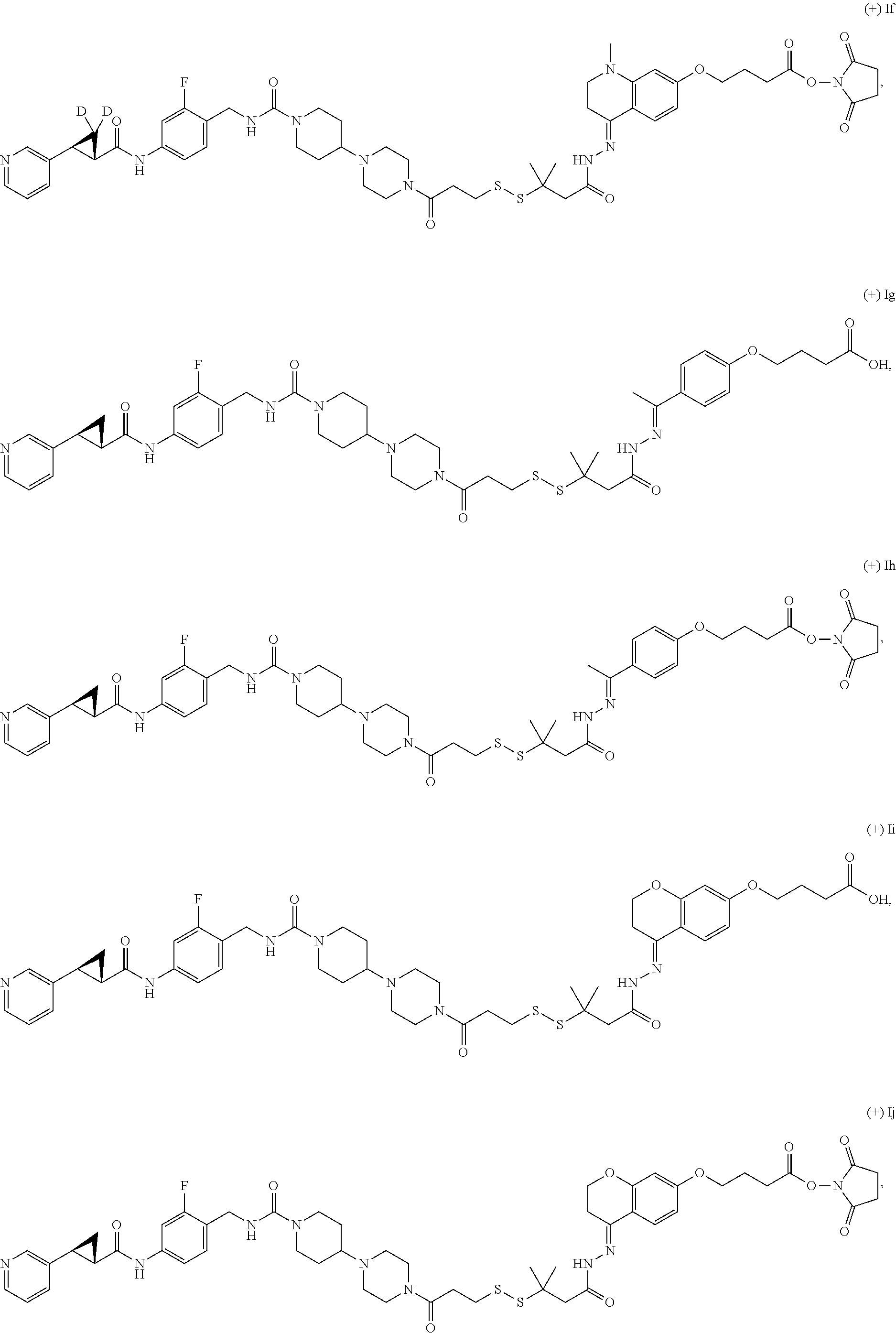

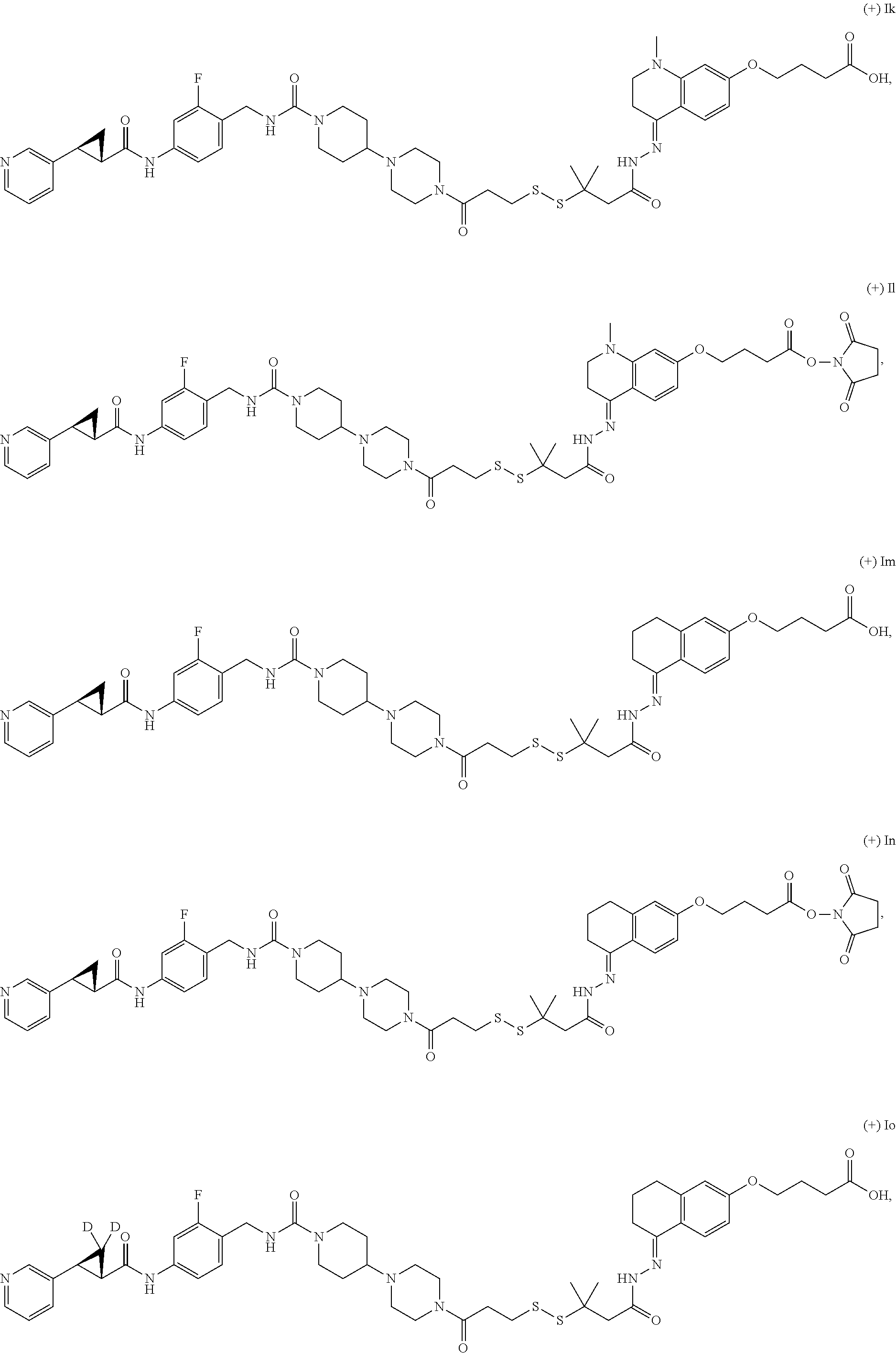

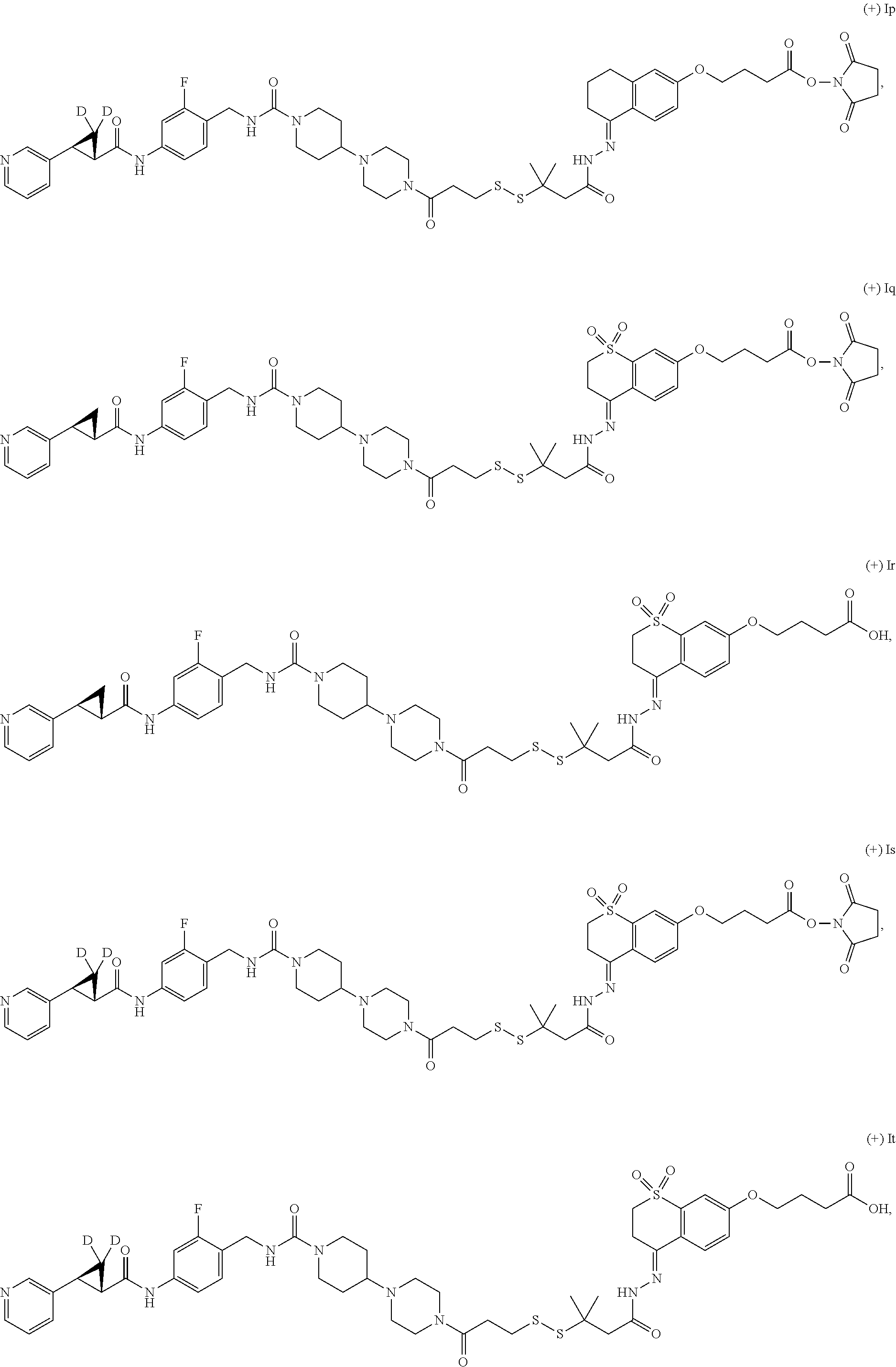

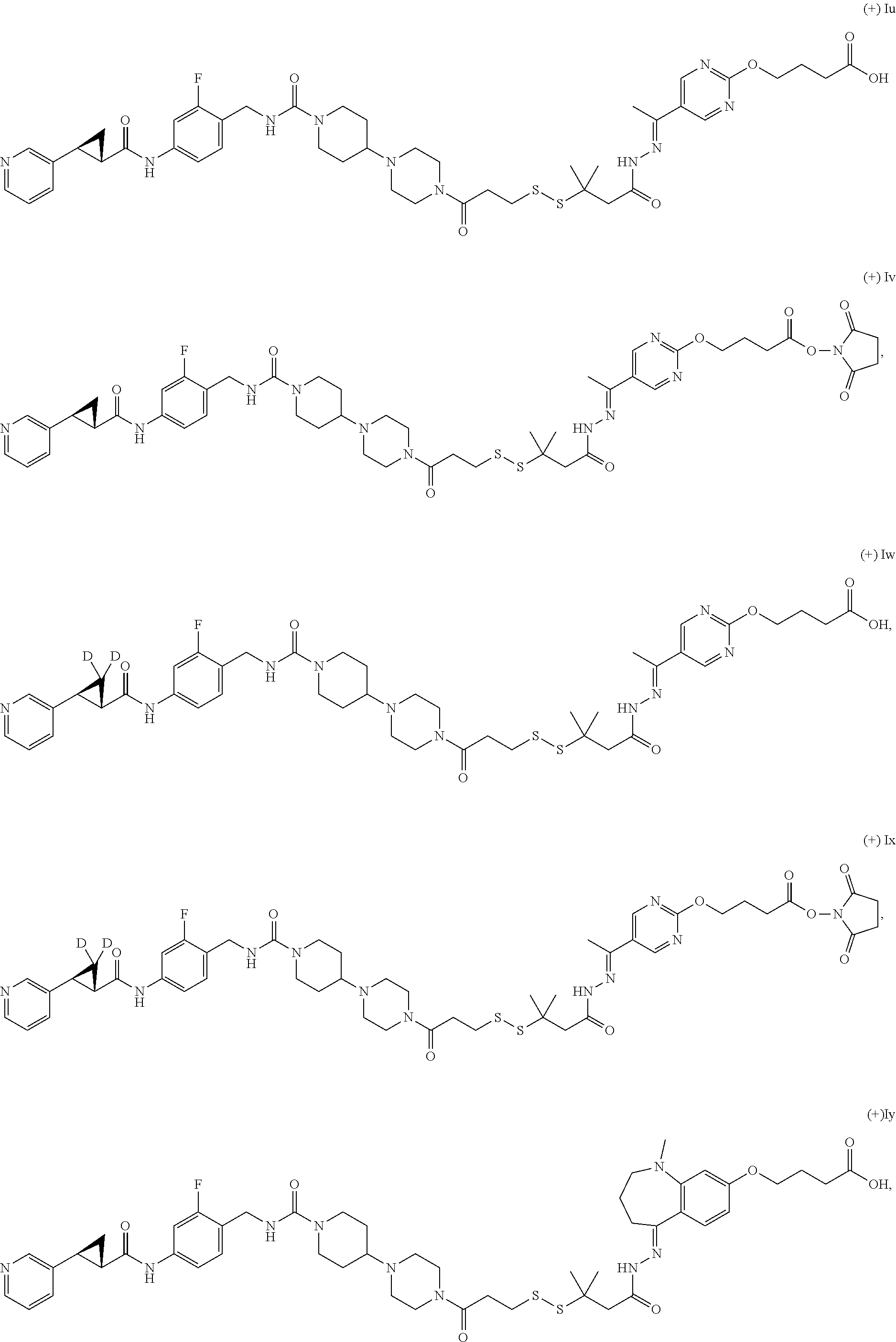

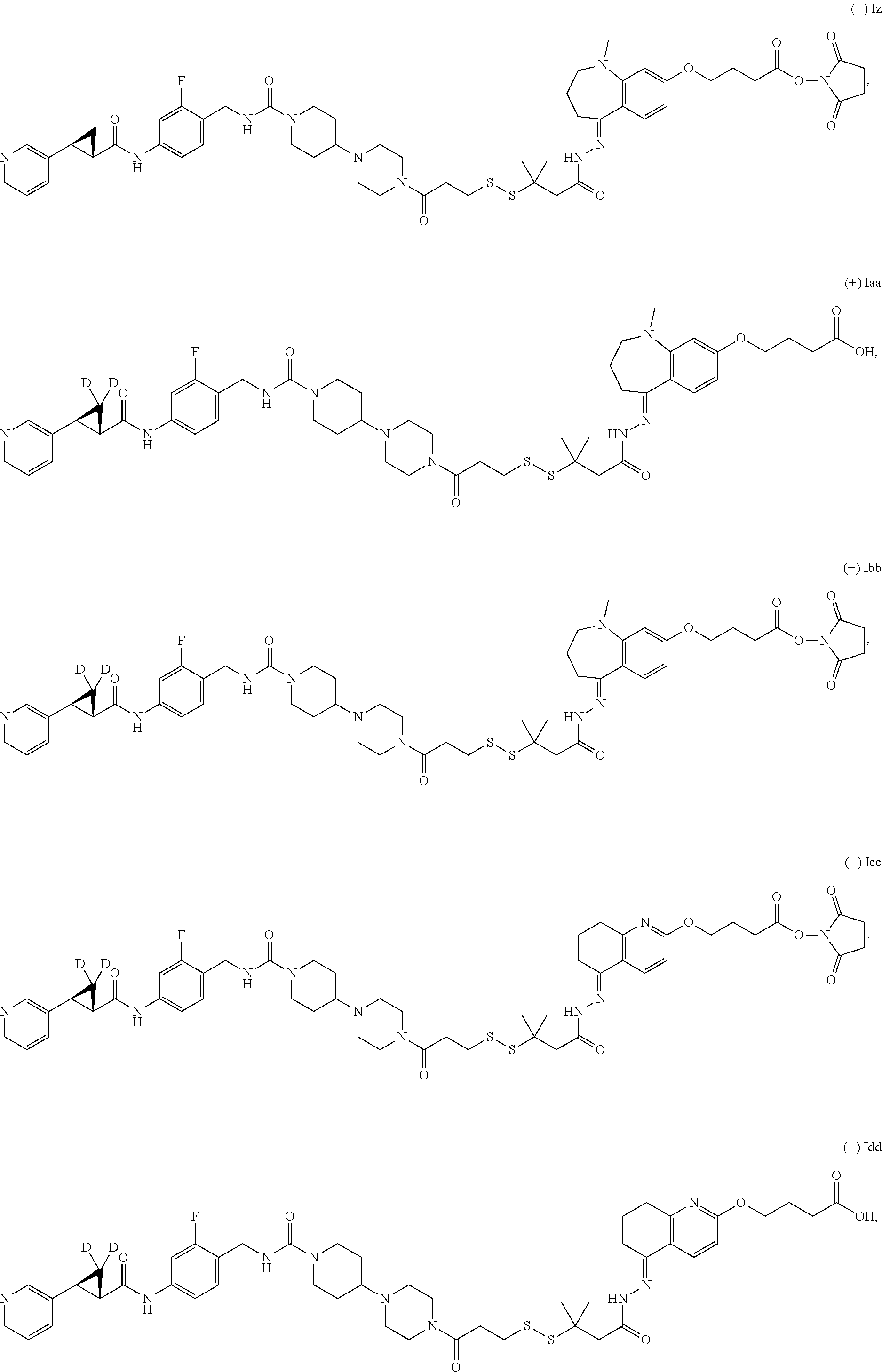

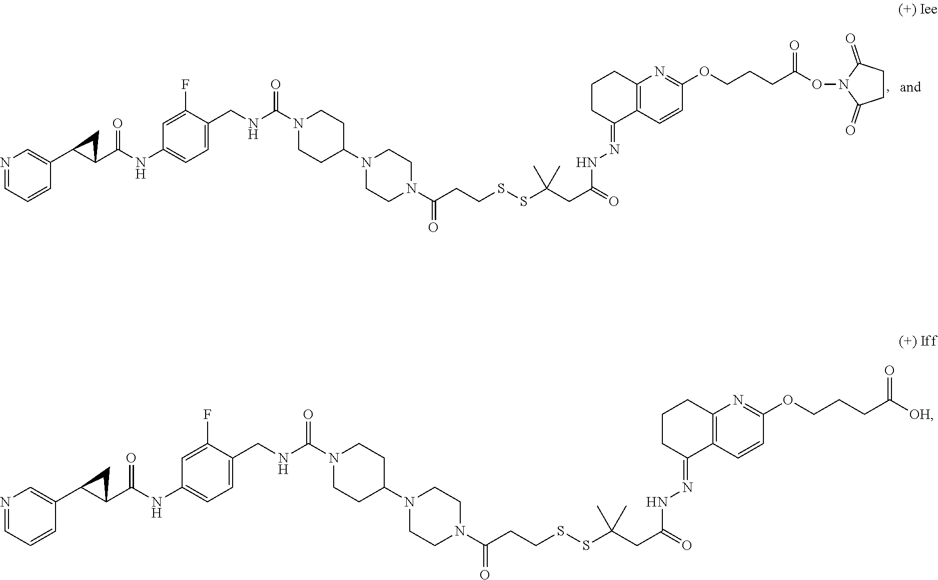 [SMILES: CC(C)(CC(=O)N/N=C1\CCCc2nc(OCCCC(=O)ON3C(=O)CCC3=O)ccc21)SSCCC(=O)N1CCN(C2CCN(C(=O)NCc3ccc(NC(=O)[C@H]4C[C@@H]4c4cccnc4)cc3F)CC2)CC1 ; CC(C)(CC(=O)N/N=C1\CCCc2nc(OCCCC(=O)O)ccc21)SSCCC(=O)N1CCN(C2CCN(C(=O)NCc3ccc(NC(=O)[C@H]4C[C@@H]4c4cccnc4)cc3F)CC2)CC1]

or a pharmaceutically acceptable salt and/or solvate thereof.

In some embodiments, the compound of Formula (I) is selected from:

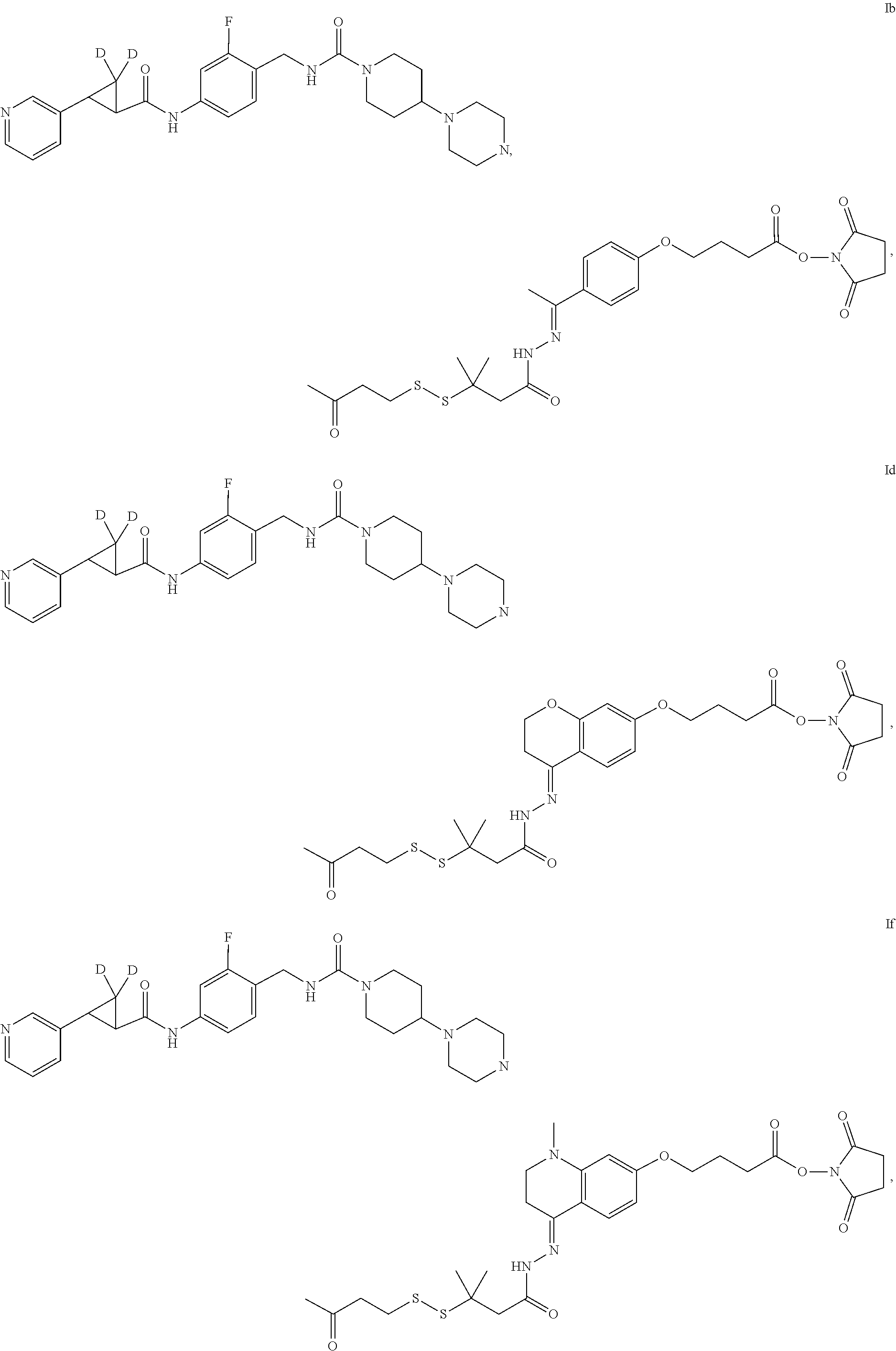

or a pharmaceutically acceptable salt and/or solvate thereof.

In some embodiments, the compound of Formula (I) is selected from:

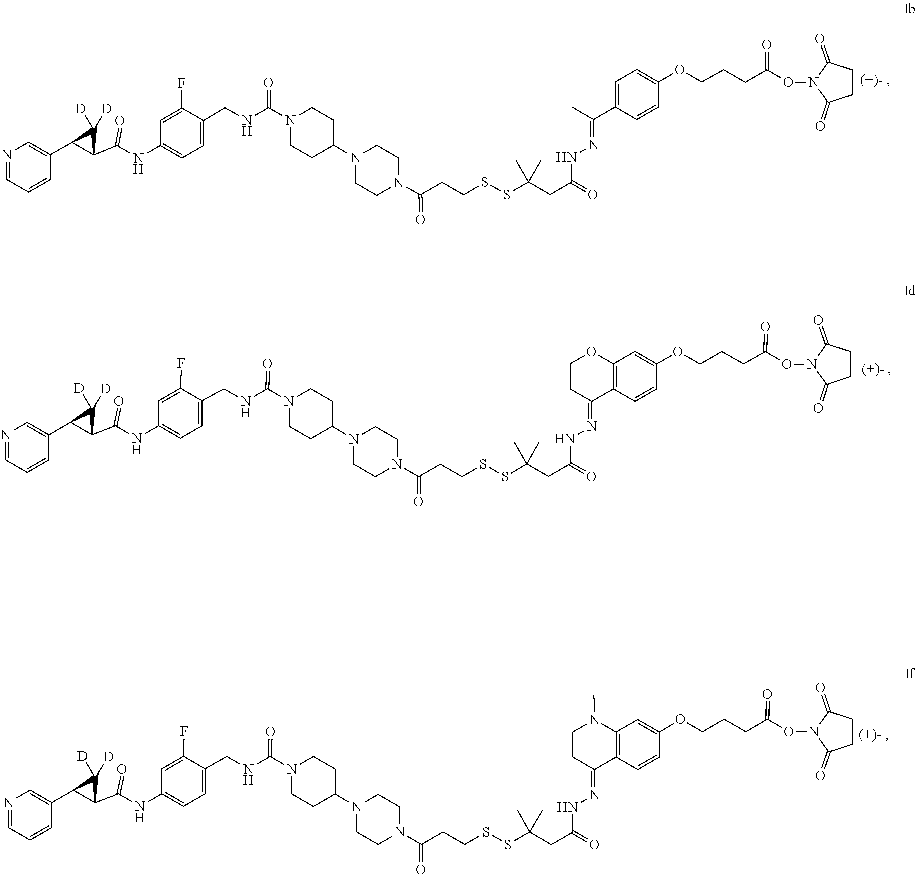 [SMILES: [2H]C1([2H])[C@H](C(=O)Nc2ccc(CNC(=O)N3CCC(N4CCN(C(=O)CCSSC(C)(C)CC(=O)N/N=C(\C)c5ccc(OCCCC(=O)ON6C(=O)CCC6=O)cc5)CC4)CC3)c(F)c2)[C@H]1c1cccnc1 ; [2H]C1([2H])[C@H](C(=O)Nc2ccc(CNC(=O)N3CCC(N4CCN(C(=O)CCSSC(C)(C)CC(=O)N/N=C5\CCOc6cc(OCCCC(=O)ON7C(=O)CCC7=O)ccc65)CC4)CC3)c(F)c2)[C@H]1c1cccnc1 ; [2H]C1([2H])[C@H](C(=O)Nc2ccc(CNC(=O)N3CCC(N4CCN(C(=O)CCSSC(C)(C)CC(=O)N/N=C5\CCN(C)c6cc(OCCCC(=O)ON7C(=O)CCC7=O)ccc65)CC4)CC3)c(F)c2)[C@H]1c1cccnc1]

or a pharmaceutically acceptable salt and/or solvate thereof.

The present application also includes a compound of Formula (II):

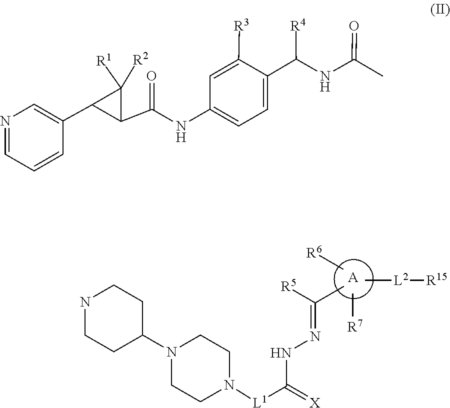

or a pharmaceutically acceptable salt and/or solvate thereof,
wherein
Ring A, L¹, L², R¹, R², R³, R⁴, R⁵, R⁶ and R⁷ are as defined above; and
R¹⁵ is a compound to be linked.

In some embodiments, R¹⁵ is selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support. In some embodiments, R¹⁵ is selected from a fluorescent dye, drug, small molecule, antibody, lipid, carbohydrate, nucleic acid, peptide, radiolabel, PET label, nanoparticle, polymer, macrocycle and metal complex.

In some embodiments, the compound of Formula (II) is for targeting a binding moiety, a labelling agent and/or a therapeutic agent to a specific site in the body of a subject. Accordingly, in some embodiments, R¹⁵ is complementary or dependent on the 2-(pyridin-3-yl)cyclopropane-1-carboxamide based nicotinamide phosphoribosyltransferase (NAMPT) inhibitor. For example, R¹⁵ is a complementary group such as a binding moiety targeting a specific site in the body (a ligand specific for a receptor or an antibody specific for an antigen) which can deliver the payload to that specific site in the body.

In some embodiments, R¹⁵ is an antibody. In some embodiments, the antibody binds to one or more tumor-associated antigens. In some embodiments, the antibody binds to one or more tumor-associated cell-surface receptors and the drug is a drug for treating cancer.

In some embodiments, the antibody is any antibody of therapeutic value. In some embodiments, the antibody is a wild type antibody amenable to cysteine or lysine conjugation. In some embodiments, the antibody is bio-engineered for site specific conjugation to enable a more controlled DAR ratio.

In some embodiments, the antibody is of the immunoglobulin (Ig) type. The immunoglobulin can be of any type (e.g., IgG, IgE, IgM, IgD and IgA), class (e.g., IgG1, IgG2, IgG3, IgG4, IgA1 and IgA2) or subclass of immunoglobulin molecule.

In some embodiments, the antibody specifically binds to a receptor encoded by an ErbB gene. In some embodiments, the antibody specifically binds to an ErbB receptor selected from EGFR, HER2, HER3 and HER4. In some embodiments, the tumor-associated cell-surface receptor is an ErbB receptor. In some embodiments, the antibody specifically binds to the EGFR receptor.

In some embodiments, the antibody specifically binds to a receptor encoded by a c-Kit gene. In some embodiments, the antibody specifically binds to a receptor encoded by a CD30 gene.

In some embodiments, the antibody is a monoclonal antibody of the IgG isotype. In some embodiments, the antibody is a chimeric antibody. In some embodiments, the antibody is selected from zalutumumab, nimotuzumab, matuzumab and cetuximab. In some embodiments, the antibody is cetuximab. In some embodiments, the antibody is trastuzumab.

In some embodiments, the drug is a drug for treating cancer. In some embodiments, the drug is selected from a protein kinase inhibitor, proteasome inhibitor, topoisomerase inhibitor, aromatase inhibitor, anthracycline, tubulin inhibitor, a nicotinamide phosphoribosyltransferase (NAMPT) inhibitor, DNA binding molecule and an alkylating agent. In some embodiments, the drug is a tubulin inhibitor. In some embodiments, the drug is monomethyl auristatin E (MMAE). In some embodiments, the drug is a macrolide. In some embodiments, the drug is a maytansinoid. In some embodiments, the drug is DM1. In some embodiments, the drug is a DNA binding agent from the pyrrolobenzodiazepine family.

In some embodiments, the drug is an anticancer drug. In some embodiments, the anticancer drug is a thiol-containing anticancer drug or a calicheamicin derivative. In some embodiments, the thiol containing anticancer drug is a maytansinoid, such as DM1. In some embodiments, the drug is a DNA binding agent selected from the pyrrolobenzodiazepine family. In some embodiments, the anticancer drug is a tubulin polymerization inhibitor. In some embodiments, the drug is MMAE.

In some embodiments, the compound of Formula II has the following structure:

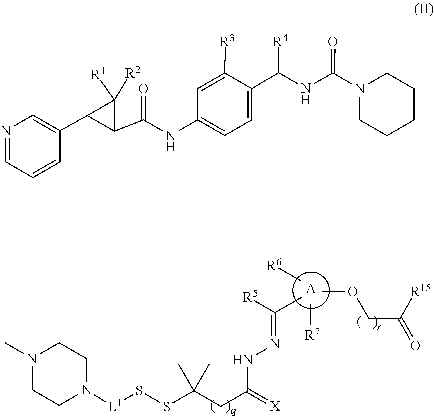

or a pharmaceutically acceptable salt and/or solvate thereof,
wherein
Ring A, L³, R¹, R², R³, R⁴, R⁵, R⁶ and R⁷ are as defined above; and
R¹⁵ is selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support;
q is 1, 2, 3, 4, 5, 6, 7 or 8; and
r is 1, 2, 3, 4, 5, 6, 7 or 8.

In some embodiments, the compound of Formula II has the following structure:

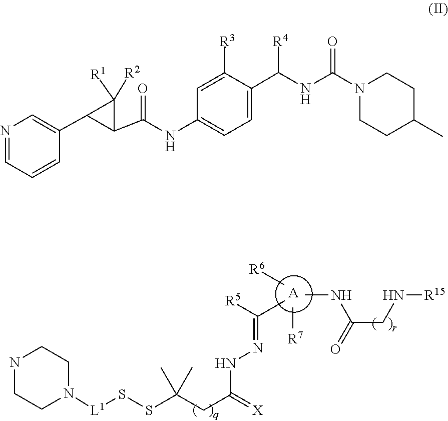

or a pharmaceutically acceptable salt and/or solvate thereof,
wherein
Ring A, L³, R¹, R², R³, R⁴, R⁵, R⁶ and R⁷ are as defined above; and
R¹⁵ is selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support;
q is 1, 2, 3, 4, 5, 6, 7 or 8; and
r is 1, 2, 3, 4, 5, 6, 7 or 8.

In some embodiments, the compound of Formula (II), or a pharmaceutically acceptable salt and/or solvate thereof, has the follow structure:

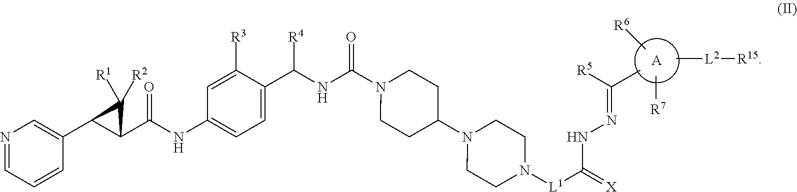

The present application includes an antibody-drug conjugate (ADC), the conjugate having a Formula (III):

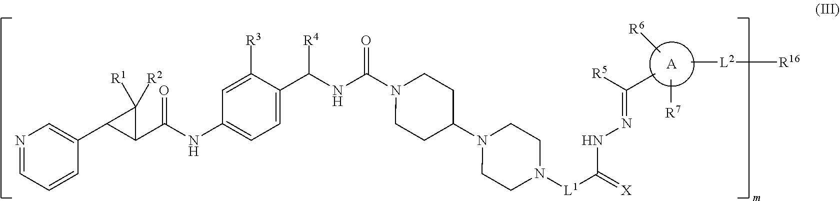

or a pharmaceutically acceptable salt and/or solvate thereof,
wherein
R¹⁶ is an antibody;
Ring A, L¹, L², R¹, R², R³, R⁴, R⁵, R⁶ and R⁷ are as defined as above; and m is an integer from 1 to 20.

In some embodiments, the compound of Formula (III) has the following structure:

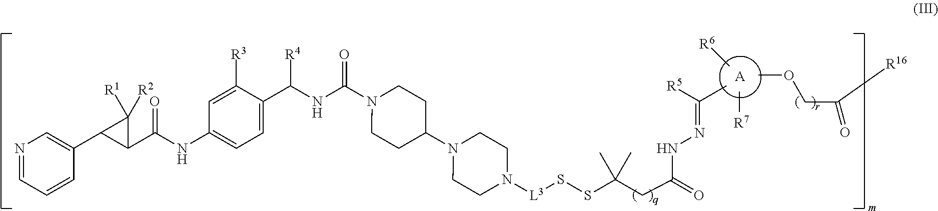

wherein
R¹⁶ is an antibody;
Ring A, L³, R¹, R², R³, R⁴, R⁵, R⁶ and R⁷ are as defined as above;
q is 1, 2, 3, 4, 5, 6, 7 or 8;
r is 1, 2, 3, 4, 5, 6, 7 or 8; and
m is an integer from 1 to 20,
or a pharmaceutically acceptable salt and/or solvate thereof.

In some embodiments, r in the compounds of Formula (III) is 2, 3 or 4. In some embodiments, r in the compounds of Formula (III) is 3. In some embodiments, q in the compounds of Formula (III) is 1 or 2. In some embodiments, q in the compounds of Formula (III) is 1. In some embodiments, R⁹ is CH₃.

In some embodiments, the compound of Formula (III) has the following structure:

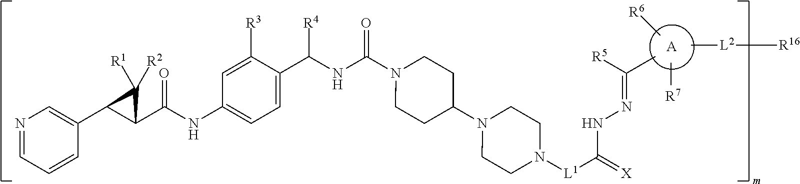

wherein
R¹⁶ is an antibody;
Ring A, L¹, L², R¹, R², R³, R⁴, R⁵, R⁶ and R⁷ are as defined as above;
q is 1, 2, 3, 4, 5, 6, 7 or 8;
r is 1, 2, 3, 4, 5, 6, 7 or 8; and
m is an integer from 1 to 20,
or a pharmaceutically acceptable salt and/or solvate thereof.

In some embodiments in the compounds of Formula (III), L³ is selected from a direct bond, Z^{b}R^{c}, Z^{b}—R^{c}, R^{c}—Z^{b}, R^{c}—Z^{b}—R^{d} and Z^{b}—R^{c}—Z^{c}, wherein Z^{b} and Z^{c} are independently selected from O, S, S(O), SO₂, NH, N(C₁₋₆alkyl), C(Q^{a}), C(Q^{a})Y^{b}, Y^{b}C(Q^{a}) Y^{b}C(Q^{a})Y^{c}, (C₁₋₆alkyleneY^{b})ₚ and Y^{b}—(C₁₋₆alkyleneY^{b})ₚ, wherein R^{c} and R^{d} are independently selected from C₁₋₁₀alkylene, C₂₋₁₀alkenylene and C₂₋₁₀alkynylene; Q^{a}, Y^{b} and Y^{c} are independently selected from O, S, NH and N(C₁₋₆alkyl); and p is selected from 1, 2, 3, 4, 5 and 6.

In some embodiments in the compounds of Formula (III), R^{c} and R^{d} are independently selected from C₁₋₆alkylene, C₂₋₆alkenylene and C₂₋₆alkynylene. In some embodiments, R^{c} and R^{d} are independently selected from C₁₋₆alkylene.

In some embodiment in the compounds of Formula (III), Q^{a}, Y^{b} and Y^{c} are independently selected from O, S, NH and N(CH₃).

In some embodiments in the compounds of Formula (III), Z^{b} and Z^{c} are independently selected from O, S, S(O), SO₂, NH, N(CH₃), C(O), C(O)NH, NHC(O), NHC(O)O, OC(O)O, NHC(O)NH, OC(O)NH, NHC(NH)NH, (C₁₋₆alkyleneO)ₚ and O—(C₁₋₆alkyleneO)ₚ,

In some embodiments, the antibody in the compounds of Formula (III), binds to one or more tumor-associated antigens. In some embodiments, the antibody binds to one or more tumor-associated cell-surface receptors. In some embodiments, the antibody specifically binds to a receptor encoded by an ErbB gene. In some embodiments, the tumor-associated cell-surface receptor is an ErbB receptor. In some embodiments, the antibody specifically binds to a receptor encoded by a c-Kit gene. In some embodiments, the tumor-associated cell-surface receptor is a c-Kit receptor. In some embodiments, the antibody specifically binds to a receptor encoded by a CD30 gene. In some embodiments, the tumor-associated cell-surface receptor is an CD30 receptor.

In some embodiments, the antibody in the compounds of Formula (III), specifically binds to an ErbB receptor selected from EGFR, HER2, HER3 and HER4. In some embodiments, the antibody specifically binds to the EGFR receptor. In some embodiments, the antibody is a monoclonal antibody. In some embodiments, the antibody is a chimeric antibody. In some embodiments, the antibody is selected from zalutumumab, nimotuzumab, matuzumab and cetuximab. In some embodiments, the antibody is cetuximab. In some embodiments, the antibody is trastuzumab.

The drug loading of ADCs is represented by the integer m, which indicates the average number of drugs conjugated per antibody in the conjugate of Formula (III). The drug to antibody (DAR) ratio is relevant for the preparation of ADC's, as higher drug loading, e.g. m>5, may cause aggregation, insolubility, toxicity or loss of cellular permeability. Further, the DAR ratio is dependent upon the number of reactive sites present on the antibody. For example, where the attachment point is a cysteine thiol or lysine amine, as in the exemplary embodiments of the present application, an antibody may have only one or few number of these reactive groups through which a linker maybe attached. Additionally, the antibody may be subjected to denaturing conditions to reveal reactive nucleophilic groups such as lysine and cysteine. In some embodiments, the DAR ratio of the compounds of Formula (III) ranges from 1 to 20 drugs per antibody.

In some embodiments, m is an integer from 1 to 10. In some embodiments, m is an integer from 1 to 5.

Known antibodies for the treatment and prevention of cancer can be conjugated as ADCs. Antibodies immunospecific for a cancer cell antigen are obtained commercially or produced by any method known to a person skilled in the art, including, e.g., chemical syntheses or by recombinant expression techniques. In some embodiments, the nucleotide sequence encoding antibodies immunospecific for a cancer cell antigens is obtained, for example, from the GenBank database or a similar nucleotide sequence database, literature publications, or through routine cloning and sequencing.

In some embodiments, the ADCs of the present application selectively deliver an effective dose of a cytotoxic agent, such as a drug, to tumor tissue with greater selectivity, i.e., a lower effective dose is achieved, than upon delivery of the same dose of drug not conjugated to an antibody.

In some embodiments, the NAMPT inhibitor drug of the compound of Formula (III) is not cleaved from the antibody until the compound enters a cell with a cell-surface receptor specific for the antibody of the compound, at which time the drug is cleaved from the antibody. In some embodiments, the drug is intracellularly cleaved from the antibody of the compound of Formula (III) through enzymatic action, hydrolysis, oxidation or pH conditions.

In some embodiments, the compound of Formula (III) is selected from:

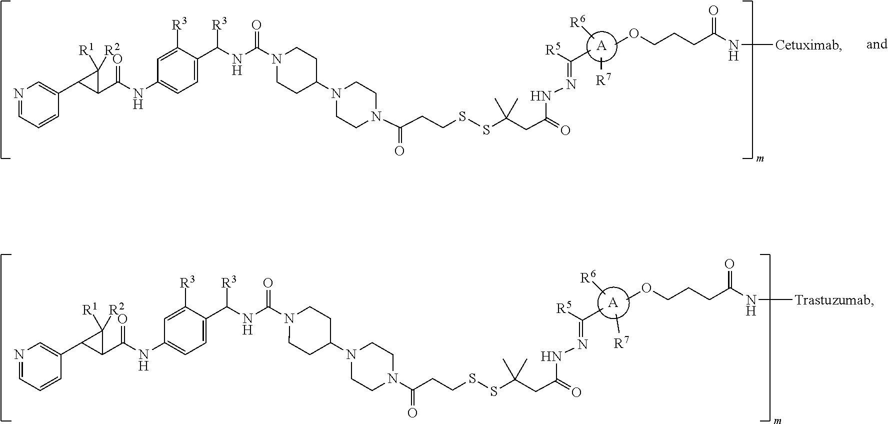

wherein
Ring A, R¹, R², R³, R⁴, R⁵, R⁶ and R⁷ are as defined as above, and
m is an integer from 1 to 20,
or a pharmaceutically acceptable salt and/or solvate thereof.

In a further aspect of the present application, a class of deuterated compounds based on a 2-(pyridin-3-yl)cyclopropane-1-carboxamide scaffold which were designed to have improved metabolic and physico-chemical properties, have been prepared.

Accordingly, the present application also includes one or more compounds of Formula (IV)

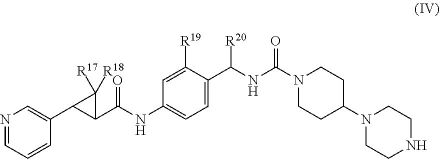

or a pharmaceutically acceptable salt and/or solvate thereof,
wherein:
R¹⁷ and R¹⁸ are independently selected from D and H;
R¹⁹ is selected from H and halo; and
R²⁰ is selected from H, C₁₋₄alkyl, and C₁₋₄fluoroalkyl.

In some embodiments, one of R¹⁷ and R¹⁸ is D and the other is H. In some embodiments, R¹⁷ and R¹⁸ are both D.

In some embodiments, R¹⁹ is selected from H and F. In some embodiments, R¹⁹ is F.

In some embodiments, R²⁰ is selected from H, CH₃ and CF₃. In some embodiments, R²⁰ is selected from CH₃ and CF₃. In some embodiments, R²⁰ is selected from CH₃ and CF₃ and the carbon atom to which it is attached has an S configuration. In some embodiments, R²⁰ is H.

In some embodiments, the compound of Formula (IV) is

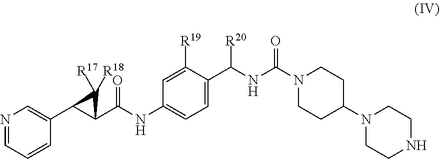

wherein
R¹⁷, R¹⁸, R¹⁹ and R²⁰ are as defined above.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may exist as mixtures of E and Z isomers or cis and trans isomers and it is intended that any above mentioned isomer, as well as mixtures thereof, are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

In some embodiments, the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolysed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like.

### III. Compositions of the Application

The compounds of the application are suitably formulated in a conventional manner into compositions using one or more carriers. Accordingly, the present application also includes a composition comprising one or more compounds of the application and a carrier. The compounds of Formula (II), (III) and/or (IV), or pharmaceutically acceptable salts and/or solvates thereof, are suitably formulated into pharmaceutical compositions for administration to subjects in a biologically compatible form suitable for administration in vivo. Accordingly, the present application further includes a pharmaceutical composition comprising one or more compounds of Formula (II), (III) and/or (IV), or pharmaceutically acceptable salts and/or solvates thereof, and a pharmaceutically acceptable carrier. In embodiments of the application the pharmaceutical compositions are used in the treatment and/or diagnosis of any of the diseases, disorders or conditions described herein.

The compounds of Formula (II), (III) and/or (IV), or pharmaceutically acceptable salts and/or solvates thereof, are administered to a subject in a variety of forms depending on the selected route of administration, as will be understood by those skilled in the art. For example, compounds of Formula (II), (III) and/or (IV), or pharmaceutically acceptable salts and/or solvates thereof, are administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, compounds of Formula (II), (III) and/or (IV), or pharmaceutically acceptable salts and/or solvates thereof, are orally administered, for example, with an inert diluent or with an assimilable edible carrier, or are enclosed in hard or soft shell gelatin capsules, or are compressed into tablets, or are incorporated directly with the food of the diet. In some embodiments, the compounds are incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compounds are protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compounds of (II), (III) and/or (IV), or pharmaceutically acceptable salts and/or solvates thereof, are suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of Formula (II), (III) and/or (IV), or pharmaceutically acceptable salts and/or solvates thereof, and use the lyophilizates obtained, for example, for the preparation of products for injection.

In some embodiments, the compounds of Formula (II), (III) and/or (IV), or pharmaceutically acceptable salts and/or solvates thereof, are administered parenterally. For example, solutions of compounds of Formula (II), (III) and/or (IV), or pharmaceutically acceptable salts and/or solvates thereof, are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of Formula (II), (III) and/or (IV), or pharmaceutically acceptable salts and/or solvates thereof, are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, compounds of (II), (III) and/or (IV), or pharmaceutically acceptable salts and/or solvates thereof, are formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of Formula (II), (III) and/or (IV), or pharmaceutically acceptable salts and/or solvates thereof, are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of compounds of Formula (II), (III) and/or (IV), or pharmaceutically acceptable salts and/or solvates thereof, and a suitable powder base such as lactose or starch. The aerosol dosage forms can also take the form of a pump-atomizer.

Compositions suitable for buccal or sublingual administration include tablets, lozenges, and pastilles, wherein compounds of Formula (II), (III) and/or (IV), or pharmaceutically acceptable salts and/or solvates thereof, are formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of Formula (II), (III) and/or (IV), or pharmaceutically acceptable salts and/or solvates thereof, are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, P A, 1980, pp. 1530-1533 for further discussion of suppository dosage forms.

In some embodiments compounds of Formula (II), or pharmaceutically acceptable salts and/or solvates thereof, are coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, compounds of Formula (II), or pharmaceutically acceptable salts and/or solvates thereof, are coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross-linked or amphipathic block copolymers of hydrogels.

The compounds of Formula (II), (III) and/or (IV), or pharmaceutically acceptable salts and/or solvates thereof, are suitably used on their own but will generally be administered in the form of a pharmaceutical composition in which the one or more compounds of Formula (II), (III) and/or (IV), or pharmaceutically acceptable salts and/or solvates thereof, (the active ingredient) are in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### IV. Methods and Uses of the Application

Compounds of Formula (II) and/or (III), or pharmaceutically acceptable salts and/or solvates thereof, comprise a wide variety of active compounds which have possibilities of treating and/or diagnosing a variety of diseases, disorders or conditions.

Accordingly, the present application includes a method of treating and/or diagnosing one or more diseases, disorders or conditions by administering an effective amount of one or more compounds of Formula (II) and/or (III), or pharmaceutically acceptable salts and/or solvates thereof, to a subject in need thereof. In some embodiments, the disease, disorder or condition depends on the identity of the compounds being conjugated as would be understood by a person skilled in the art.

In some embodiments, the disease, disorder or condition is a neoplastic disorder. Accordingly, the present application also includes a method of treating and/or diagnosing a neoplastic disorder comprising administering a therapeutically effective amount of one or more compounds of Formula (II) and/or (III), or pharmaceutically acceptable salts and/or solvates thereof, to a subject in need thereof. The present application also includes a use of one or more compounds of Formula (II) and/or (III), or pharmaceutically acceptable salts and/or solvates thereof, for treatment of and/or diagnosing a neoplastic disorder as well as a use of one or more compounds of Formula (II) and/or (III), or pharmaceutically acceptable salts and/or solvates thereof, for the preparation of a medicament for treatment of and/or diagnosing a neoplastic disorder. The application further includes one or more compounds of Formula (II) and/or (III), or pharmaceutically acceptable salts and/or solvates thereof, for use in treating and/or diagnosing a neoplastic disorder. In an embodiment, the treatment is in an amount effective to ameliorate at least one symptom of the neoplastic disorder, for example, reduced cell proliferation or reduced tumor mass, among others, in a subject in need of such treatment.

In some embodiments, the present application includes a method of treating and/or diagnosing one or more diseases, disorders or conditions mediated by ErbB comprising administering a therapeutically effective amount of one or more compounds of Formula (II) and/or (III), or pharmaceutically acceptable salts and/or solvates thereof, to a subject in need thereof. The present application also includes a use of one or more compounds of Formula (II) and/or (III), or pharmaceutically acceptable salts and/or solvates thereof, for treatment of and/or diagnosing one or more diseases, disorders or conditions mediated by ErbB as well as a use of one or more compounds of Formula (II) and/or (III), or pharmaceutically acceptable salts and/or solvates thereof, for the preparation of a medicament for treatment of and/or diagnosing one or more diseases, disorders or conditions mediated by ErbB.

In some embodiments, the disease, disorder or condition is cancer. Accordingly, the present application also includes a method of treating and/or diagnosing cancer comprising administering a therapeutically effective amount of one or more compounds of Formula (II) and/or (III), or pharmaceutically acceptable salts and/or solvates thereof, to a subject in need thereof. The present application also includes a use of one or more compounds of Formula (II) and/or (III), or pharmaceutically acceptable salts and/or solvates thereof, for treatment of and/or diagnosing cancer as well as a use of one or more compounds of Formula (II) and/or (III), or pharmaceutically acceptable salts and/or solvates thereof, for the preparation of a medicament for treatment of and/or diagnosing cancer. The application further includes one or more compounds of Formula (II) and/or (III), or pharmaceutically acceptable salts and/or solvates thereof, for use in treating cancer. In an embodiment, the compound is administered for the prevention of cancer in a subject such as a mammal having a predisposition for cancer. In some embodiments, the cancer is an ErbB-expressing cancer, c-Kit-expressing cancer or a CD30-expressing cancer. In some embodiments, the subject is human.

The compounds of Formula (II) and/or (III) inhibit nicotinamide phosphoribosyltransferase (NAMPT) activity.

Accordingly, the present application includes a method for inhibiting NAMPT in a cell, either in a biological sample or in a patient, comprising administering an effective amount of one or more compounds of Formula (II) and/or (III) to the cell. The application also includes a use of one or more compounds of Formula (II) and/or (III) for inhibiting NAMPT in a cell as well as a use of one or more compounds of Formula (II) and/or (III) for the preparation of a medicament for inhibiting NAMPT in a cell. The application further includes one or more compounds of Formula (II) and/or (III) for use in inhibiting NAMPT in a cell.

The present application also includes a method of treating a disease, disorder or condition by inhibition of NAMPT comprising administering a therapeutically effective amount of one or more compounds of Formula (II) and/or (III) to a subject in need thereof.

The present application also includes a use of one or more compounds of Formula (II) and/or (III) for treatment of a disease, disorder or condition by inhibition of NAMPT as well as a use of one or more compounds of Formula (II) and/or (III) for the preparation of a medicament for treatment of a disease, disorder or condition by inhibition of NAMPT. The application further includes one or more compounds of Formula (II) and/or (III) for use in treating a disease, disorder or condition by inhibition of NAMPT.

In a further aspect of the present application, the compounds of Formula (IV) have been shown to inhibit nicotinamide phosphoribosyltransferase (NAMPT) activity.

Accordingly, the present application includes a method for inhibiting NAMPT in a cell, either in a biological sample or in a patient, comprising administering an effective amount of one or more compounds of Formula (IV) to the cell. The application also includes a use of one or more compounds of Formula (IV) for inhibiting NAMPT in a cell as well as a use of one or more compounds of Formula (IV) for the preparation of a medicament for inhibiting NAMPT in a cell. The application further includes one or more compounds of Formula (IV) for use in inhibiting NAMPT in a cell.

As the compounds of Formula (IV) have been shown to inhibit NAMPT protein activity, the compounds of Formula (IV) are useful for treating diseases, disorders or conditions by inhibiting NAMPT. Therefore the compounds of Formula (IV) are useful as medicaments. Accordingly, the present application includes a compound of Formula (IV) for use as a medicament.

The present application also includes a method of treating a disease, disorder or condition by inhibition of NAMPT comprising administering a therapeutically effective amount of one or more compounds of Formula (IV) to a subject in need thereof.

The present application also includes a use of one or more compounds of Formula (IV) for treatment of a disease, disorder or condition by inhibition of NAMPT as well as a use of one or more compounds of Formula (IV) for the preparation of a medicament for treatment of a disease, disorder or condition by inhibition of NAMPT. The application further includes one or more compounds of Formula (IV) for use in treating a disease, disorder or condition by inhibition of NAMPT.

In an embodiment, the disease, disorder or condition is a neoplastic disorder. Accordingly, the present application also includes a method of treating a neoplastic disorder comprising administering a therapeutically effective amount of one or more compounds of Formula (IV) to a subject in need thereof. The present application also includes a use of one or more compounds of Formula (IV) for treatment of a neoplastic disorder as well as a use of one or more compounds of the application for the preparation of a medicament for treatment of a neoplastic disorder. The application further includes one or more compounds of Formula (IV) for use in treating a neoplastic disorder. In an embodiment, the treatment is in an amount effective to ameliorate at least one symptom of the neoplastic disorder, for example, reduced cell proliferation or reduced tumor mass, among others, in a subject in need of such treatment.

In another embodiment of the present application, the disease, disorder or condition that is treated by inhibition of NAMPT is cancer. Accordingly, the present application also includes a method of treating cancer comprising administering a therapeutically effective amount of one or more compounds of Formula (IV) to a subject in need thereof. The present application also includes a use of one or more compounds of Formula (IV) for treatment of cancer as well as a use of one or more compounds of Formula (IV) for the preparation of a medicament for treatment of cancer. The application further includes one or more compounds of Formula (IV) for use in treating cancer. In an embodiment, the compound is administered for the prevention of cancer in a subject such as a mammal having a predisposition for cancer. In some embodiments, the cancer is an ErbB-expressing cancer or a c-Kit-expressing cancer. In some embodiments, the subject is human.

Neoplasms can be benign (such as uterine fibroids and melanocytic nevi), potentially malignant (such as carcinoma in situ) or malignant (i.e. cancer). Exemplary neoplastic disorders include the so-called solid tumours and liquid tumours, including but not limited to carcinoma, sarcoma, metastatic disorders (e.g., tumors arising from the prostate), hematopoietic neoplastic disorders, (e.g., leukemias, lymphomas, myeloma and other malignant plasma cell disorders), metastatic tumors and other cancers.

In some embodiments, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macroglobulinemia; and Wilms' Tumor. Metastases of the aforementioned cancers can also be treated in accordance with the methods described herein.

In some embodiments, the cancer is selected from ErbB-expressing cancer. In some embodiments, the cancer is selected from breast cancer, skin cancer, prostate cancer, head and neck cancer, colorectal cancer, pancreatic cancer, kidney cancer, lung cancer and brain cancer. In some embodiments of the present application, the cancer is selected from breast cancer, prostate cancer, head and neck cancer, colorectal cancer, pancreatic cancer, kidney cancer, lung cancer and brain cancer.

In a further embodiment, the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies.

In some embodiments, when the methods and uses are related to diagnostics, one compound to be linked comprises a binding moiety and the other compound to be linked comprises a labelling agent.

In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In an embodiment, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In an embodiment, the subject is a mammal. In another embodiment, the subject is human.

The compounds of Formula (II), (III) and/or (IV), or pharmaceutically acceptable salts and/or solvates thereof, are either used alone or in combination with other known agents useful for treatment and/or imaging. When used in combination with other agents useful in treatment and/or imaging, it is an embodiment that compounds of Formula (II), (III) and/or (IV), or pharmaceutically acceptable salts and/or solvates thereof, are administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, compounds of Formula (II), (III) and/or (IV), or pharmaceutically acceptable salts and/or solvates thereof, are administered with another therapeutic agent simultaneously or sequentially in separate unit dosage forms or together in a single unit dosage form. Accordingly, the present application provides a single unit dosage form comprising one or more compounds of Formula (II), (III) and/or (IV), or pharmaceutically acceptable salts and/or solvates thereof, an additional therapeutic agent, and a pharmaceutically acceptable carrier.

In some embodiments, the additional therapeutic agent is a chemotherapeutic agent. In some embodiments, the chemotherapeutic agent is selected from the classes of alkylating agents, anthracyclines, cytoskeletal disruptors, epothilones, histone deacetylase inhibitors, topoisomerase inhibitors, kinase inhibitors, nucleotide analogs, peptide antibiotics, platinum-based agents, retinoids, Vinca alkaloids, epigenetic modifiers and immuno-modulators.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, a compound of the application is administered initially in a suitable dosage that is adjusted as required, depending on the clinical response. Dosages will generally be selected to maintain a serum level of the compound of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg.

### V. Methods of Preparing Compounds of the Application

Scheme 1 illustrates one embodiment of a route to compounds of Formula (I) in which a functionalized hydrazide is formed from commercially available compounds A, wherein R⁸ is a reactive functional group or a protected form thereof and X and L¹ are as defined in Formula (I) to afford intermediates B. The subsequent coupling of B with aromatic compounds C, wherein Ring A, R⁵, R⁶, R⁷, R⁸ and L² are as defined in Formula II and in which R¹¹ may be in protected form, provides compounds of the application.

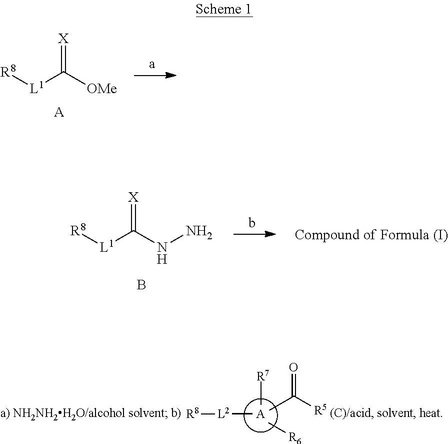

Compounds of Formula C are either commercially available or are synthesized from commercially available compounds using methods known in the art, for example starting from compounds of Formula D:

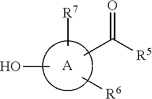

wherein Ring A, R⁶, R⁷, and R⁸ are as defined in Formula (I).

In some embodiments, the reactive functional group R⁸ of the compounds of Formula I are subsequently conjugated to a complementary reactive functional group of compounds to be linked, for example, a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex or solid support, to produce the compounds of Formula (II) or (III) of the present application.

Accordingly, in another aspect, the present application includes a method of synthesizing one or more compounds of Formula (II) or (III) as defined above, or a pharmaceutically acceptable salt and/or solvate thereof, wherein the method comprises reacting one or more compounds of Formula (I) as defined above with a compound to be linked, for example, selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex or solid support.

For preparing ADC compounds of Formula (III) of the application, in some embodiments, a compound of Formula (I) is first prepared. Methods for conjugating a Formula (I) to an antibody and purifying the ADCs are known to those skilled in the art.

Accordingly, in another aspect the present application includes a method of preparing an ADC of Formula (III) comprising: (a) reacting a compound of Formula (I) with an antibody to provide the ADC of Formula (III); and optionally (c) purifying the ADC of Formula (III).

The present application also includes a use of a compound of Formula (I) to prepare an ADC.

In some embodiments, the resulting ADC products are isolated or purified using known methods, such as for example, lyophilization, chromatography, precipitation, filtration, microfluidic and/or liquid chromatography separation methods.

In some embodiments, compounds of Formula (IV) or pharmaceutically acceptable salt and/or solvate thereof, are prepared using methods known in the art.

In some embodiments, compounds of Formula (IV) or pharmaceutically acceptable salt and/or solvate thereof, are prepared according to Scheme 2. Therefore, a 2-(pyridin-3-yl)cyclopropane-1-carboxylic acid compound of formula E is coupled with with an amino compound of Formula F wherein PG is a protecting group under suitable coupling conditions such as in the presence of active ester forming reagents (e.g., hexafluorophosphate azabenzotriazole tetramethyl uranium, HATU) and a base (e.g., N,N-diisopropylethylaminediethylamine, DIEA) in a suitable solvent (e.g. dimethyl formamide, DMF). Subsequent deprotection of the resulting material provides compounds of Formula (IV).

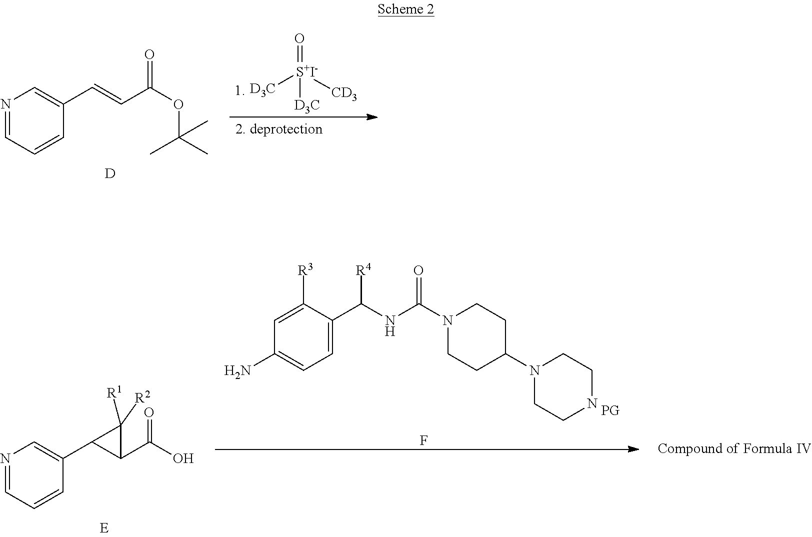

Compounds of Formula E are synthesized from commercially available compounds, for example starting from compounds of Formula D in the presence of a suitable methylene transfer reagent such as trimethylsulfoxonium iodide.

The present application also includes a method of preparing a cyclopropyl compound of Formula E wherein R¹ and R² are both H, or R¹ and R² are both D, by reacting a compound of Formula D with trimethylsulfoxonium iodide or trimethylsulfoxonium-d₉ iodide.

In some embodiments, compounds of Formula (IV) are subsequently conjugated with a complementary reactive functional group of a suitable linker compounds to form drug-linker conjugates of Formula (I).

In some embodiments, compounds of Formula (I) to (IV) comprising deuterium are prepared according to the processes illustrated in the schemes above, with deuterium being incorporated through commercially available deuterated agents. For example, a compound of Formula E wherein R¹ and R² are both D is prepared by reacting a compound of Formula D in the presence of trimethyl sulfoxonium-d₉-iodide.

### EXAMPLES

The following non-limiting examples are illustrative of the present application:

### A. General Methods

Exemplary compounds of the application were synthesized using the methods described herein, or other methods, which are known in the art. Unless otherwise noted, reagents and solvents were obtained from commercial suppliers (e.g. Aldrich, Enamine, Combi-Blocks, Bepharm, J&W PharmLab,).

The compounds and/or intermediates were characterized by high performance liquid chromatography (HPLC) using a Waters ACQUITY™ UPLC system with a SQ (single quadrupole) MS and a photodiode array (PDA) detector (Milford, MA). The analytical columns were reversed phase Acquity UPLC BEH C₁₈ (2.1×50 mm, 1.7 μm). A gradient elution was used (flow 0.4 mL/min), typically starting with mobile phase 0.1% formic acid in water (solvent A) and 0.1% formic acid in acetonitrile (solvent B). A gradient starting at 95% solvent A going to 5% in 1.8 min., holding for 0.5 min., going back to 95% in 0.5 min. and equilibrating the column for 0.5 min. Compounds were detected by ultraviolet light (UV) absorption at either 220 or 254 nm. HPLC solvents were from Burdick and Jackson (Muskegan, MI), or Fisher Scientific (Pittsburgh, PA).

In some instances, purity was assessed by thin layer chromatography (TLC) using glass or plastic backed silica gel plates, such as, for example, Baker-Flex Silica Gel IB2-F flexible sheets. TLC results were readily detected visually under ultraviolet light, or by employing well-known iodine vapor and other various staining techniques

The compounds and/or intermediates were characterized by LCMS. General conditions are as follows. Low and High resolution Mass spectra were acquired on LC/MS systems using electrospray ionization methods from a range of instruments of the following configurations: Low resolution—Waters ACQUITY™ UPLC system with a SQ (single quadrupole) MS; Waters ACQUITY™ UPLC H-Class system with a 3100 (single quadrupole) MS. High resolution—Waters ACQUITY UPLC II system equipped with a Synapt Xevo QTof and Waters ACQUITY UPLC II system equipped with a Synapt G2S QTof mass spectrometer with an atmospheric pressure ionization source. [M+H] refers to the protonated molecular ion of the chemical species.

Nuclear magnetic resonance (NMR) analysis was performed on a Bruker 500 MHz NMR spectrometer using ICON-NMR, under TopSpin program control. Spectra were measured at 298K, unless indicated otherwise and were referenced relative to the solvent chemical shift. ¹H NMR spectra were processed using ACD Labs Spectrus software.

### B. Synthesis of Compounds of the Application

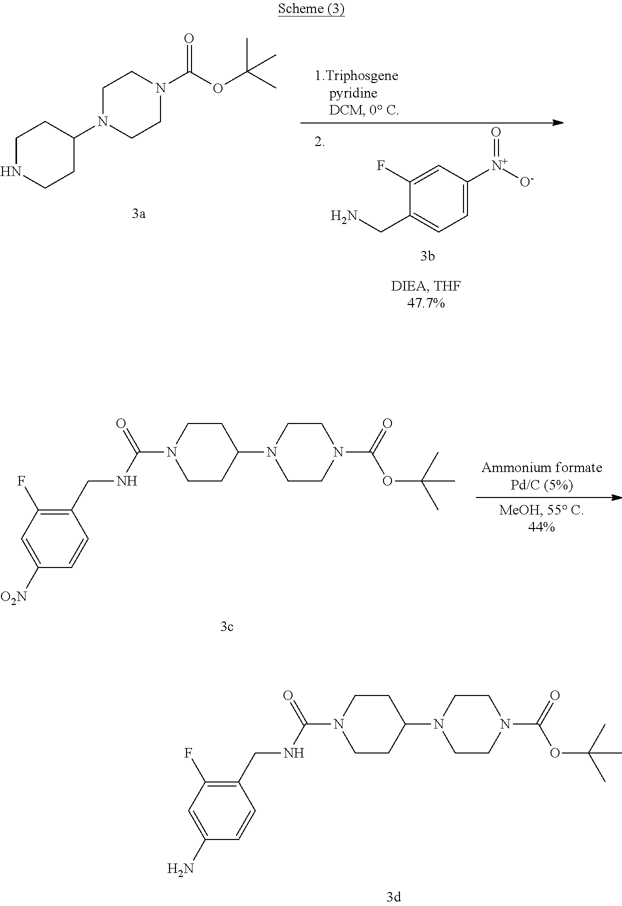 [SMILES: CC(C)(C)OC(=O)N1CCN(C2CCNCC2)CC1 ; NCc1ccc([N+](=O)[O-])cc1F ; CC(C)(C)OC(=O)N1CCN(C2CCN(C(=O)NCc3ccc([N+](=O)[O-])cc3F)CC2)CC1 ; CC(C)(C)OC(=O)N1CCN(C2CCN(C(=O)NCc3ccc(N)cc3F)CC2)CC1]

### tert-Butyl 4-(1-((2-fluoro-4-nitrobenzyl)carbamoyl)piperidin-4-yl)piperazine-1-carboxylate (3c)

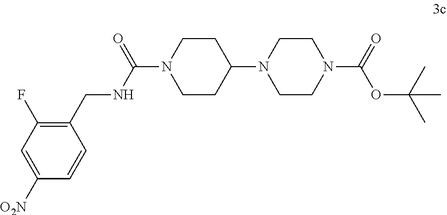 [SMILES: CC(C)(C)OC(=O)N1CCN(C2CCN(C(=O)NCc3ccc([N+](=O)[O-])cc3F)CC2)CC1]

To a cooled solution (0° C.) of triphosgene (0.463 g, 1.559 mmol) in 20 mL dichloromethane is added pyridine (0.359 mL, 4.45 mmol). 1-Boc-4-(piperidin-4-yl)-piperazine 3a (1.2 g, 4.45 mmol) is added portionwise and it was stirred at 0° C. for about 30 min then the ice bath was removed. After 2 h at RT, the reaction mixture was diluted with EtOAc, washed with water (×3) then brine. It was dried over Na₂SO₄ and concentrated down. The residue was dried further under high vacuum to afford the intermediate carbamoyl chloride as a white solid (720 mg). Crude amine 3b (1.24 g) was dissolved in DCM (15 mL) then N,N-diisopropylethylamine (2.328 mL, 13.36 mmol) was added. While stirring, a solution of the above carbamoyl chloride (720 mg in 10 mL of DCM) was added with a pipette. The mixture was stirred under N₂ for 2 days upon which LCMS showed most of the carbamoyl chloride was consumed and the major component was the right product. Celite was added to the mixture then it was dried. It was purified using CombiFlash^{RF} by reverse phase chromatography (13 g C₁₈ column: eluent 10-50% then 50% acetonitrile/water) to afford the title product 3c as a very light yellow foam (990 mg, 47.7% yield). ¹H NMR (CHLOROFORM-d, 500 MHz) δ 8.03 (dd, 1H, J=1.9, 8.5 Hz), 7.93 (dd, 1H, J=2.2, 9.7 Hz), 7.62 (t, 1H, J=7.9 Hz), 5.00 (br t, 1H, J=5.8 Hz), 4.55 (d, 2H, J=5.9 Hz), 4.01 (br d, 2H, J=13.2 Hz), 3.4-3.5 (m, 4H), 2.8-2.9 (m, 2H), 2.51 (br s, 4H), 2.4-2.5 (m, 1H), 1.85 (br d, 2H, J=11.7 Hz), 1.48 (s, 9H); LCMS [M+H]⁺ 466.

### tert-Butyl 4-(1-((4-amino-2-fluorobenzyl)carbamoyl)piperidin-4-yl)piperazine-1-carboxylate (3d)

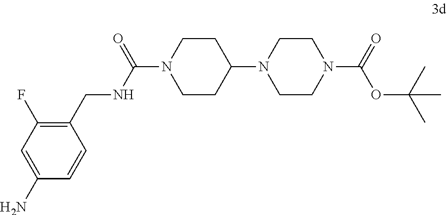 [SMILES: CC(C)(C)OC(=O)N1CCN(C2CCN(C(=O)NCc3ccc(N)cc3F)CC2)CC1]

To a 100 mL RB flask containing tert-butyl 4-(1-((2-fluoro-4-nitrobenzyl)carbamoyl)piperidin-4-yl)piperazine-1-carboxylate 3c (790 mg, 1.697 mmol) was added MeOH (30 mL). The solution was stirred then purged with N₂. Ammonium formate (2140 mg, 33.9 mmol) was added during this process followed by Pd/C (5%) (200 mg). The mixture was heated at 55° C. in an oil bath under a gentle stream of N₂ for 2 h. It was cooled down and filtered through a pad of celite. The filter cake was washed several times with MeOH/EtOAc (1/1) and concentrated down. The residue was separated between water and EtOAc. The organic layer was washed with water, brine then dried over Na₂SO₄. It was concentrated down to give the title compound 3d as a tan solid (325 mg, 44% yield). ¹H NMR (DMSO-d6, 500 MHz) δ 6.93 (t, 1H, J=8.6 Hz), 6.77 (br t, 1H, J=5.5 Hz), 6.31 (dd, 1H, J=2.1, 8.2 Hz), 6.26 (dd, 1H, J=2.1, 12.7 Hz), 5.22 (s, 2H), 4.08 (d, 2H, J=5.5 Hz), 4.00 (br d, 2H, J=13.0 Hz), 3.28 (br s, 4H), 2.6-2.6 (m, 2H), 2.41 (br s, 4H), 1.68 (br d, 2H, J=11.9 Hz), 1.40 (s, 9H); LCMS [M+H]⁺ 436.

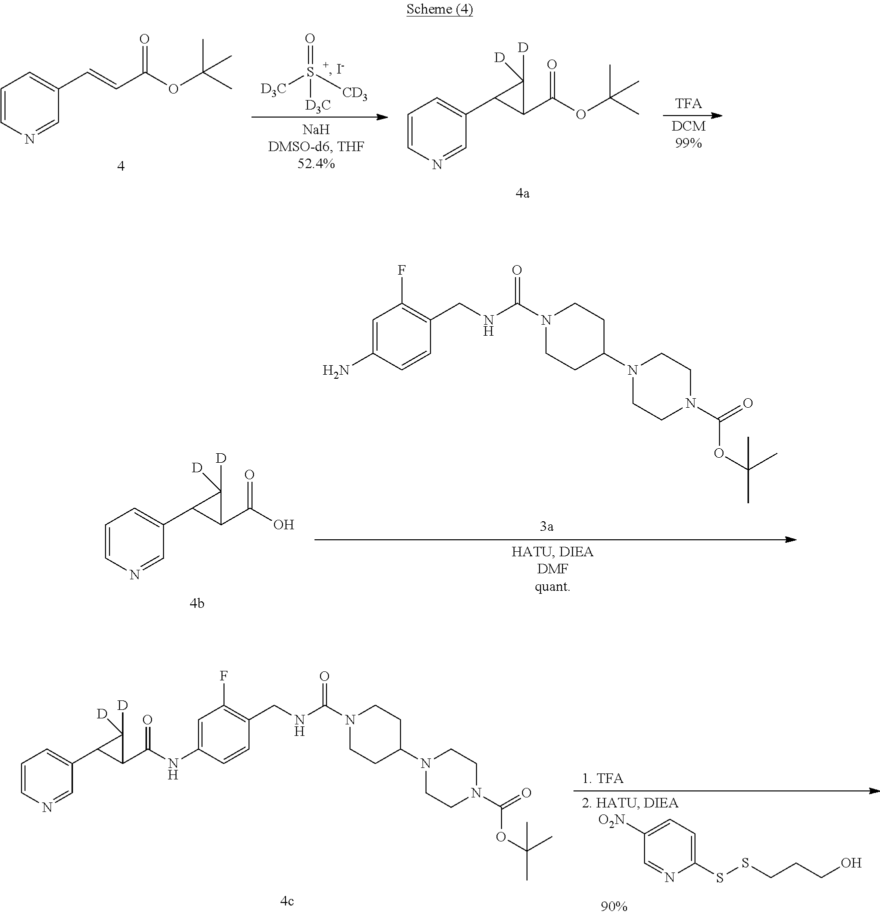

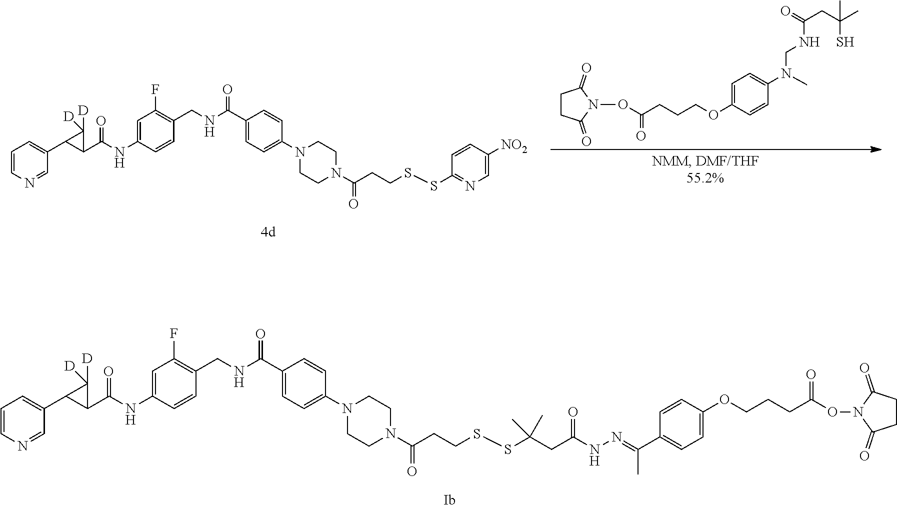 [SMILES: [2H]C1([2H])C(C(=O)Nc2ccc(CNC(=O)c3ccc(N4CCN(C(=O)CCSSc5ccc([N+](=O)[O-])cn5)CC4)cc3)c(F)c2)C1c1cccnc1 ; CN(CNC(=O)CC(C)(C)S)c1ccc(OCCCC(=O)ON2C(=O)CCC2=O)cc1 ; [2H]C1([2H])C(C(=O)Nc2ccc(CNC(=O)c3ccc(N4CCN(C(=O)CCSSC(C)(C)CC(=O)N/N=C(\C)c5ccc(OCCCC(=O)ON6C(=O)CCC6=O)cc5)CC4)cc3)c(F)c2)C1c1cccnc1]

### tert-Butyl 2-(pyridin-3-yl)cyclopropane-1-carboxylate-3,3-d2 (4a)

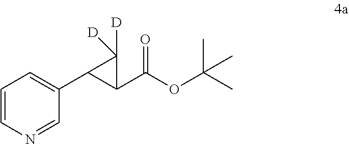 [SMILES: [2H]C1([2H])C(C(=O)OC(C)(C)C)C1c1cccnc1]

Sodium hydride, 60% in mineral oil (5.46 g, 136 mmol) was added to a solution of anhydrous d6-dimethylsulfoxide (30 mL) and THE (60 mL). The mixture was heated to 70° C. for 30 min then cooled down to 0° C. Trimethylsulfoxonium-d9 iodide (29.0 g, 127 mmol) was added upon which the solution was vigorously stirred for 10 min. A solution of tert-butyl (E)-3-(pyridin-3-yl)acrylate 4 (8 g, 39 mmol) in THE (30 mL) was added. The flask containing 4 was washed with THE (5 mL) and added to the mixture then it was stirred at RT for about 6 h. LCMS showed only a small amount of SM 4 remaining. The reaction mixture was cooled to 0° C. then carefully quenched with a saturated solution of ammonium chloride. EtOAc and water were added then the layers were separated. The organic layer was washed with water (×3) then brine. It was dried over sodium sulfate overnight. It was concentrated down and the resulting crude was adsorbed onto celite and dried. It was purified using CombiFlash^{RF} (120 g Gold silica column; eluent, 0%, 0-25%, 25% then 50% EtOAc/hexanes) to afford the title compound 4a as a light orange oil (4.517 g, 52.4% yield). ¹H NMR (CHLOROFORM-d, 500 MHz) δ 8.4-8.5 (m, 2H), 7.35 (td, 1H, J=1.8, 7.9 Hz), 7.21 (dd, 1H, J=4.9, 7.8 Hz), 2.4-2.5 (m, 1H), 1.86 (d, 1H, J=4.0 Hz), 1.49 (s, 9H); LCMS [M+H]+ 222.

### 2-(Pyridin-3-yl)cyclopropane-1-carboxylic-3,3-d2 acid (4b)

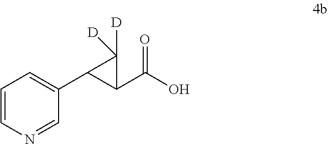 [SMILES: [2H]C1([2H])C(C(=O)O)C1c1cccnc1]

To a 100 mL RB flask containing tert-butyl 2-(pyridin-3-yl)cyclopropane-1-carboxylate-3,3-d2 4a (4.505 g, 20.36 mmol) was added DCM (20 mL) followed by TFA (20 mL). The mixture was stirred at RT for 2 h upon which LCMS showed almost completion. The solvent was removed in the high vacuum rotavap. It was co-evaporated twice with toluene then with MeOH to remove any residual TFA. It was dried under vacuum to get the desired product 4b as an-off white solid (5.632 g, 99% yield, TFA salt). ¹H NMR (DMSO-d6, 500 MHz) δ 11.8-13.1 (m, 1H), 8.68 (d, 1H, J=2.0 Hz), 8.59 (dd, 1H, J=1.2, 5.2 Hz), 7.97 (br d, 1H, J=8.1 Hz), 7.65 (dd, 1H, J=5.3, 7.9 Hz), 2.6-2.6 (m, 1H), 2.01 (d, 1H, J=4.2 Hz); LCMS LCMS [M+H]+ 166.

### tert-Butyl 4-(1-((2-fluoro-4-(2-(pyridin-3-yl)cyclopropane-1-carboxamido-3,3-d2)benzyl)carbamoyl)piperidin-4-yl)piperazine-1-carboxylate (4c)

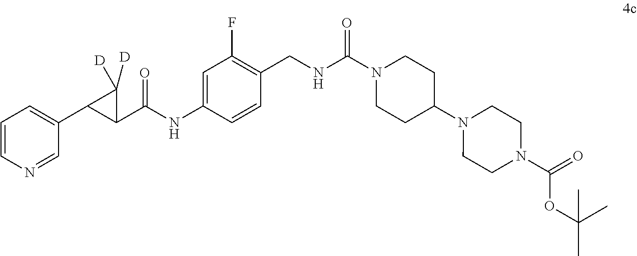 [SMILES: [2H]C1([2H])C(C(=O)Nc2ccc(CNC(=O)N3CCC(N4CCN(C(=O)OC(C)(C)C)CC4)CC3)c(F)c2)C1c1cccnc1]

To a 30 mL vial containing tert-butyl 4-(1-((4-amino-2-fluorobenzyl)carbamoyl)piperidin-4-yl)piperazine-1-carboxylate 3a (318 mg, 0.731 mmol), was added 2-(pyridin-3-yl)cyclopropane-1-carboxylic-3,3-d2 acid, TFA salt 4b (170 mg, 0.609 mmol) and HATU (301 mg, 0.792 mmol). It was dissolved in dry DMF (5 mL) then the mixture was stirred at RT for 10 min. N,N-Diisopropylethylamine (0.530 mL, 3.04 mmol) was added. The mixture was stirred at RT for 1 h upon which LCMS showed completion. It was diluted with EtOAc and washed with water. An emulsion was formed upon shaking. It was broken with some brine. This was repeated for a total of 3 times. It was then washed with brine and dried over Na₂SO₄. It was concentrated down to afford the crude title compound 4c as a beige foamy solid (418 mg, quant. yield). ¹H NMR (DMSO-d6, 500 MHz) δ 10.42 (s, 1H), 8.50 (d, 1H, J=1.6 Hz), 8.42 (dd, 1H, J=1.2, 4.6 Hz), 7.6-7.6 (m, 1H), 7.55 (s, 1H), 7.32 (dd, 1H, J=4.7, 7.9 Hz), 7.2-7.2 (m, 2H), 6.97 (t, 1H, J=5.7 Hz), 4.20 (br d, 2H, J=5.5 Hz), 4.00 (br d, 2H, J=12.6 Hz), 3.28 (br s, 5H), 3.18 (d, 1H, J=5.3 Hz), 2.6-2.7 (m, 2H), 2.42 (br d, 5H, J=3.9 Hz), 2.10 (d, 1H, J=4.2 Hz), 1.69 (br d, 2H, J=10.3 Hz), 1.39 (s, 9H); LCMS [M+H]⁺ 583.

### N-(2-Fluoro-4-(2-(pyridin-3-yl)cyclopropane-1-carboxamido-3,3-d2)benzyl)-4-(4-(3-((5-nitropyridin-2-yl)disulfaneyl)propanoyl)piperazin-1-yl)benzamide (4d)

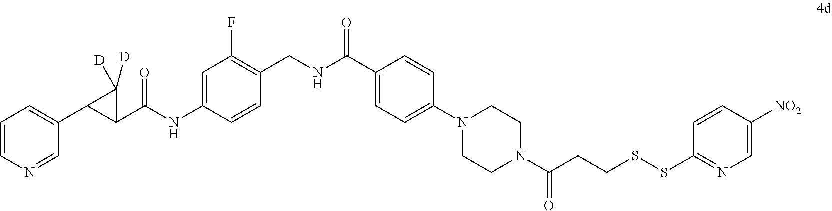 [SMILES: [2H]C1([2H])C(C(=O)Nc2ccc(CNC(=O)c3ccc(N4CCN(C(=O)CCSSc5ccc([N+](=O)[O-])cn5)CC4)cc3)c(F)c2)C1c1cccnc1]

tert-Butyl 4-(1-((2-fluoro-4-(2-(pyridin-3-yl)cyclopropane-1-carboxamido-3,3-d2)benzyl)carbamoyl)piperidin-4-yl)piperazine-1-carboxylate 4c (416 mg, 0.714 mmol) was dissolved in DCM (3 mL) then TFA (3 mL) was added. The mixture was stirred at RT upon which LCMS showed completion. The volatiles were evaporated down. The residual TFA was co-evaporated twice with toluene. It was dried under high vacuum to afford the deprotected product as a light brown foamy solid (768 mg). To a 100 mL RB flask containing this latter product (764 mg) was added 3-((5-nitropyridin-2-yl)disulfanyl)propanoic acid (162 mg, 0.622 mmol) and HATU (373 mg, 0.982 mmol). DMF (10 mL) was added then the mixture was stirred at RT for 10 min upon which N,N-diisopropylethylamine (1.369 mL, 7.86 mmol) was added. The mixture was stirred at RT for 30 min at which point LCMS showed complete conversion. The mixture was diluted with EtOAc and washed with water (×3) then brine. It was dried over Na₂SO₄ and concentrated down. It was dried further under high vacuum, partially solubilized in acetonitrile and lyophilized to get the title compound 4d as a light orange fluffy powder (428 mg, 90% yield). ¹H NMR (DMSO-d6, 500 MHz) δ 10.42 (s, 1H), 9.27 (d, 1H, J=2.6 Hz), 8.59 (dd, 1H, J=2.7, 8.9 Hz), 8.50 (d, 1H, J=1.8 Hz), 8.42 (dd, 1H, J=1.3, 4.6 Hz), 8.04 (d, 1H, J=8.9 Hz), 7.57 (br d, 1H, J=2.1 Hz), 7.55 (s, 1H), 7.32 (dd, 1H, J=4.8, 7.8 Hz), 7.22 (s, 1H), 7.21 (s, 1H), 7.19 (br s, 1H), 6.97 (br t, 1H, J=5.5 Hz), 4.20 (br d, 2H, J=5.3 Hz), 4.00 (br d, 3H, J=12.6 Hz), 3.09 (t, 2H, J=6.5 Hz), 2.76 (t, 2H, J=6.5 Hz), 2.6-2.7 (m, 3H), 2.42 (br d, 7H, J=4.0 Hz), 2.09 (d, 1H, J=4.0 Hz), 1.68 (br d, 2H, J=12.0 Hz), 1.2-1.3 (m, 4H); LCMS [M+H]⁺ 726.

Linker-drug construct (Ib)

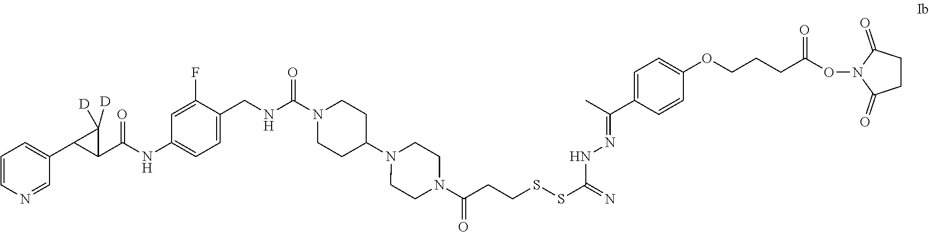 [SMILES: [2H]C1([2H])C(C(=O)Nc2ccc(CNC(=O)N3CCC(N4CCN(C(=O)CCSSC(=N)N/N=C(\C)c5ccc(OCCCC(=O)ON6C(=O)CCC6=O)cc5)CC4)CC3)c(F)c2)C1c1cccnc1]

N-(2-Fluoro-4-(2-(pyridin-3-yl)cyclopropane-1-carboxamido-3,3-d2)benzyl)-4-(4-(3-((5-nitropyridin-2-yl)disulfanyl)propanoyl)piperazin-1-yl)piperidine-1-carboxamide 4d (30.4 mg, 0.042 mmol) was dissolved in DMF (1.5 mL) then 2,5-dioxopyrrolidin-1-yl-4-(4-(1-(2-(3-mercapto-3-methylbutanoyl)hydrazono)ethyl)phenoxy)butanoate (37.7 mg, 0.084 mmol) in THF (4.1 mL) was added. 4-Methylmorpholine (0.084 mL, 0.042 mmol) as a 0.5M solution in DMF was added. The mixture was stirred at room temperature for 10 min upon which LCMS showed complete conversion. The crude mixture was separated between water and EtOAc and shaken. The organic layer was washed with water (×3) then brine. It was dried over Na₂SO₄ and concentrated down. The crude was purified over CombiFlash^{RF} (4 g Gold silica column; eluent: EtOAc/hexanes; 0-100% then 100% EtOAc followed by acetone/EtOAc 0-100% then 100%). The product was taken into acetonitrile frozen then lyophilized to afford the title compound Ia as a white fluffy powder (24.8 mg, 55.2% yield, 2 isomers). ¹H NMR (DMSO-d6, 500 MHz) δ 10.42 (s, 1H), 10.28 (s, 1H), 8.50 (d, 1H, J=2.1 Hz), 8.42 (dd, 1H, J=1.5, 4.8 Hz), 7.77 (d, 1H, J=8.9 Hz), 7.74 (d, 1H, J=8.9 Hz), 7.32 (dd, 1H, J=4.8, 7.8 Hz), 7.2-7.2 (m, 2H), 6.9-7.0 (m, 3H), 4.20 (br d, 2H, J=5.4 Hz), 4.10 (dt, 2H, J=2.6, 6.2 Hz), 4.00 (br d, 2H, J=11.6 Hz), 3.41 (br s, 4H), 3.06 (s, 1H), 2.95 (br t, 2H, J=6.7 Hz), 2.86 (td, 2H, J=3.7, 7.2 Hz), 2.82 (br s, 4H), 2.7-2.7 (m, 3H), 2.64 (s, 2H), 2.4-2.4 (m, 6H), 2.23 (s, 1H), 2.21 (s, 2H), 2.12 (s, 1H), 2.1-2.1 (m, 3H), 1.67 (br t, 2H, J=9.0 Hz), 1.42 (s, 6H); LCMS [M+H]+ 1019.

Linker-drug construct (Id)

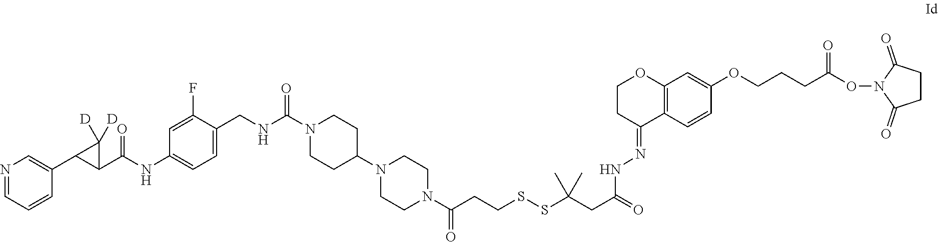 [SMILES: [2H]C1([2H])C(C(=O)Nc2ccc(CNC(=O)N3CCC(N4CCN(C(=O)CCSSC(C)(C)CC(=O)N/N=C5\CCOc6cc(OCCCC(=O)ON7C(=O)CCC7=O)ccc65)CC4)CC3)c(F)c2)C1c1cccnc1]

The title compound Id was prepared using similar procedures to Ib. It was collected as an off-white fluffy powder (20.8 mg, 53.7% yield, 2 isomers). ¹H NMR (DMSO-d6, 500 MHz) δ 10.49 (s, 1H), 10.42 (s, 1H), 10.33 (s, 1H), 8.50 (d, 1H, J=1.5 Hz), 8.4-8.4 (m, 1H), 7.8-7.9 (m, 1H), 7.58 (br dd, 1H, J=1.7, 3.7 Hz), 7.55 (s, 1H), 7.32 (dd, 1H, J=4.8, 7.8 Hz), 7.2-7.2 (m, 2H), 6.97 (br d, 1H, J=2.4 Hz), 6.64 (ddd, 1H, J=2.3, 8.9, 16.9 Hz), 6.4-6.5 (m, 1H), 4.56 (s, 1H), 4.24 (br d, 1H, J=7.9 Hz), 4.2-4.2 (m, 3H), 4.0-4.1 (m, 2H), 4.00 (br d, 2H, J=12.0 Hz), 3.41 (br s, 4H), 3.06 (s, 1H), 2.9-3.0 (m, 2H), 2.84 (br s, 2H), 2.82 (br s, 4H), 2.78 (q, 2H, J=6.4 Hz), 2.69 (br t, 2H, J=7.1 Hz), 2.65 (br d, 1H, J=1.6 Hz), 2.6-2.6 (m, 2H), 2.4-2.4 (m, 5H), 2.12 (s, 1H), 2.09 (d, 1H, J=3.9 Hz), 2.0-2.1 (m, 2H), 1.67 (br d, 2H, J=5.0 Hz), 1.42 (d, 6H, J=2.6 Hz), 1.24 (br s, 3H), 1.15 (s, 3H); LCMS [M+H]⁺ 1046.

Linker-drug construct (I)

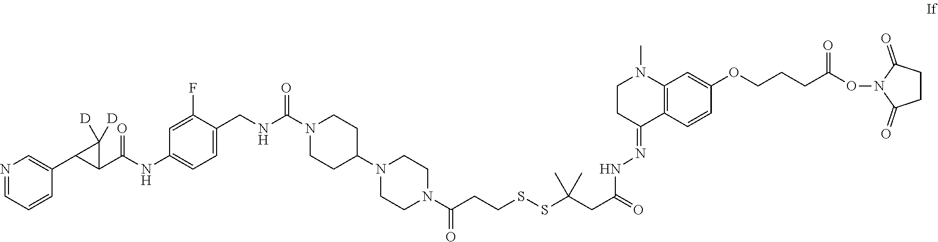 [SMILES: [2H]C1([2H])C(C(=O)Nc2ccc(CNC(=O)N3CCC(N4CCN(C(=O)CCSSC(C)(C)CC(=O)N/N=C5\CCN(C)c6cc(OCCCC(=O)ON7C(=O)CCC7=O)ccc65)CC4)CC3)c(F)c2)C1c1cccnc1]

The title compound If was prepared using similar procedures to Ib. It was collected as a light beige fluffy powder (9.2 mg, 32.7% yield, 2 isomers).

¹H NMR (DMSO-d6, 500 MHz) δ 10.42 (s, 1H), 10.28 (s, 1H), 10.17 (s, 1H), 8.50 (d, 1H, J=2.0 Hz), 8.42 (dd, 1H, J=1.5, 4.6 Hz), 7.8-7.9 (m, 1H), 7.5-7.6 (m, 2H), 7.32 (dd, 1H, J=4.8, 7.9 Hz), 7.2-7.2 (m, 2H), 6.96 (br d, 1H, J=2.9 Hz), 6.38 (ddd, 1H, J=2.2, 8.8, 18.3 Hz), 6.2-6.3 (m, 1H), 4.20 (br d, 2H, J=5.4 Hz), 4.0-4.1 (m, 2H), 4.00 (br d, 3H, J=11.4 Hz), 3.41 (br s, 4H), 3.16 (td, 2H, J=6.5, 15.9 Hz), 3.04 (s, 1H), 2.95 (q, 2H, J=6.7 Hz), 2.8-2.9 (m, 3H), 2.84 (br s, 2H), 2.82 (br s, 4H), 2.7-2.7 (m, 5H), 2.65 (br d, 2H, J=1.6 Hz), 2.41 (br s, 2H), 2.39 (br d, 3H, J=6.7 Hz), 2.12 (s, 1H), 2.0-2.1 (m, 3H), 1.6-1.7 (m, 2H), 1.42 (s, 6H), 1.24 (br s, 6H); LCMS [M+H]⁺ 1060.

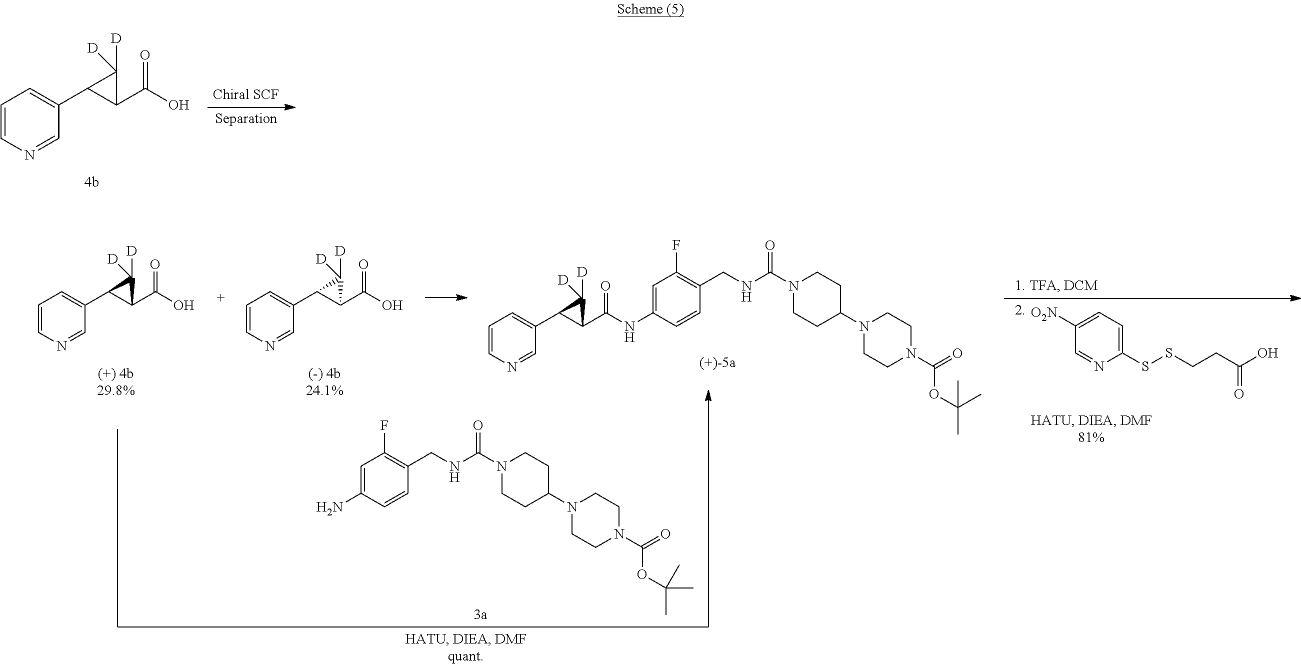 [SMILES: [2H]C1([2H])C(C(=O)O)C1c1cccnc1 ; [2H]C1([2H])[C@H](C(=O)O)[C@H]1c1cccnc1 ; [2H]C1([2H])[C@@H](C(=O)O)[C@@H]1c1cccnc1 ; CC(C)(C)OC(=O)N1CCN(C2CCN(C(=O)NCc3ccc(N)cc3F)CC2)CC1 ; [2H]C1([2H])[C@H](C(=O)Nc2ccc(CNC(=O)N3CCC(N4CCN(C(=O)OC(C)(C)C)CC4)CC3)c(F)c2)[C@H]1c1cccnc1 ; O=C(O)CCSSc1ccc([N+](=O)[O-])cn1]

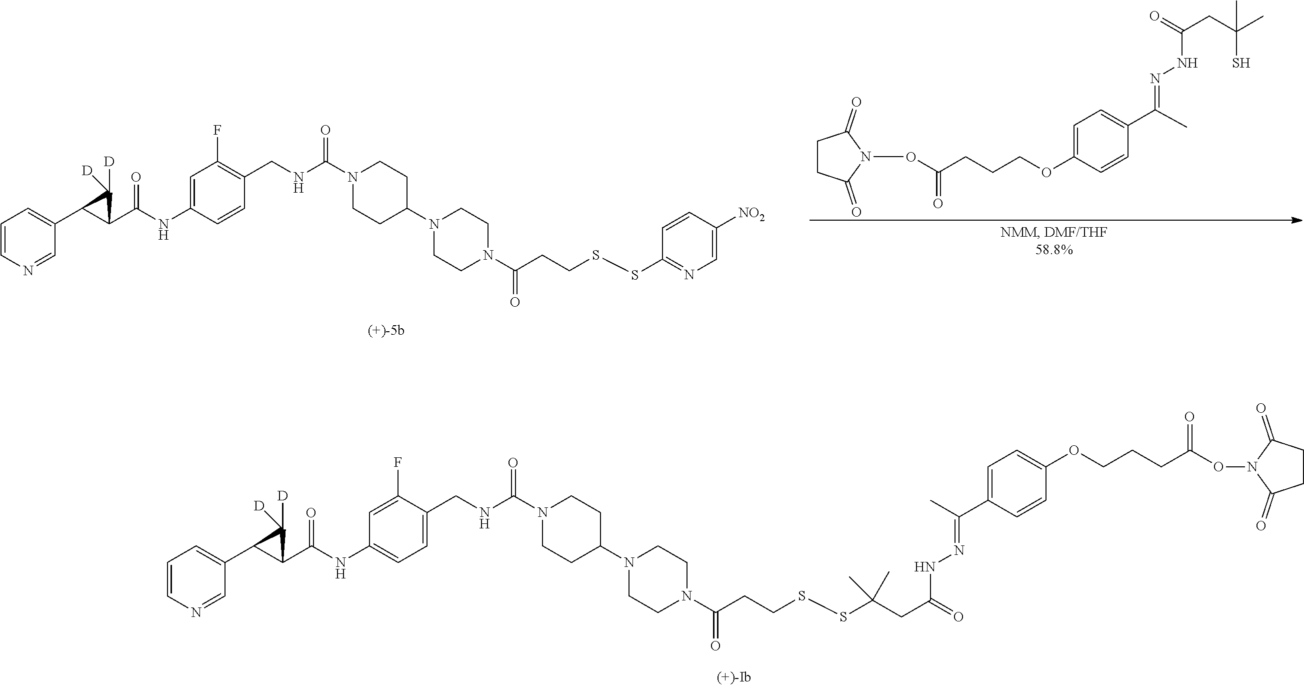 [SMILES: [2H]C1([2H])[C@H](C(=O)Nc2ccc(CNC(=O)N3CCC(N4CCN(C(=O)CCSSc5ccc([N+](=O)[O-])cn5)CC4)CC3)c(F)c2)[C@H]1c1cccnc1 ; C/C(=N\NC(=O)CC(C)(C)S)c1ccc(OCCCC(=O)ON2C(=O)CCC2=O)cc1 ; [2H]C1([2H])[C@H](C(=O)Nc2ccc(CNC(=O)N3CCC(N4CCN(C(=O)CCSSC(C)(C)CC(=O)N/N=C(\C)c5ccc(OCCCC(=O)ON6C(=O)CCC6=O)cc5)CC4)CC3)c(F)c2)[C@H]1c1cccnc1]

Chiral separation of racemic (4b):

Racemic 4b (5.266 g) was separated using chiral preparative supercritical fluid chromatography (SFC). Preparative SFC Conditions: Instrument: SFC-PIC-002, column/dimensions: Chiralpak AD-H (4.6×250 mm) 5μ, CO₂: 70.0%, co-solvent (MeOH): 30.0%, total flow: 100.0 g/ml, back pressure: 120 bar, UV: 214 nm, stack time: 6.3 min.

This separation afforded the two enantiomers (+) 4b (1.57 g, 29.8%) and (−) 4b (1.27 g, 24.1%).

### (1S,2S)-2-(Pyridin-3-yl)cyclopropane-1-carboxylic-3,3-d2 acid (+) 4b

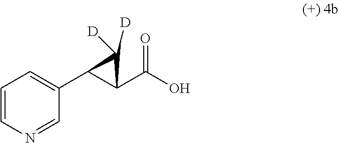 [SMILES: [2H]C1([2H])[C@H](C(=O)O)[C@H]1c1cccnc1]

¹H NMR (400 MHz, CDCl3): δ 8.53 (s, 1H), δ 8.46 (d, J=4.4 Hz, 1H), δ 7.85-7.78 (m, 1H), δ 7.58-7.50 (m, 1H), δ 2.58 (d, J=3.6 Hz, 1H), δ 1.98 (d, J=4 Hz, 1H); LCMS [M+H]+ 166; [α]_{D}=+146.8 (c: 1% in MeOH).

### (1R,2R)-2-(pyridin-3-yl)cyclopropane-1-carboxylic-3,3-d2 acid (−) 4b

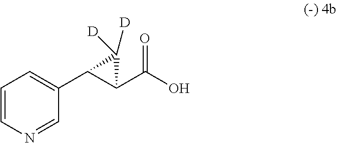 [SMILES: [2H]C1([2H])[C@@H](C(=O)O)[C@@H]1c1cccnc1]

¹H NMR (400 MHz, CDCl3): δ 8.43 (d, J=1.2 Hz, 1H), δ 8.37 (d, J=3.6 Hz, 1H), δ 7.63-7.56 (m, 1H) δ 7.40-7.33 (m, 1H), δ 2.52 (d, J=4 Hz, 1H), δ 1.92 (d, J=4 Hz, 1H); LCMS [M+H]+ 166; [α]_{D}=−153.6 (c: 1% in MeOH).

### tert-Butyl4-(1-((2-fluoro-4-((1S,2S)-2-(pyridin-3-yl)cyclopropane-1-carboxamido-3,3-d2)benzyl)carbamoyl)piperidin-4-yl)piperazine-1-carboxylate (+) 5a

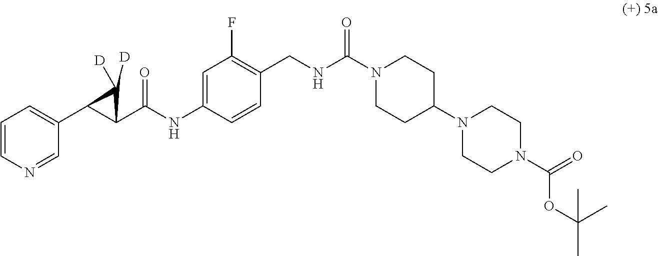 [SMILES: [2H]C1([2H])[C@H](C(=O)Nc2ccc(CNC(=O)N3CCC(N4CCN(C(=O)OC(C)(C)C)CC4)CC3)c(F)c2)[C@H]1c1cccnc1]

To a 30 mL vial containing tert-butyl4-(1-((4-amino-2-fluorobenzyl)carbamoyl)piperidin-4-yl)piperazine-1-carboxylate 3a (318 mg, 0.731 mmol), was added (1S, 2S)-2-(pyridin-3-yl)cyclopropane-1-carboxylic-3,3-d2 acid (+) 4b (173 mg, 0.620 mmol) and HATU (325 mg, 0.855 mmol). It was dissolved in dry DMF (5 mL) then mixture was stirred at RT for 10 min. N,N-Diisopropylethylamine (0.540 mL, 3.10 mmol) was added then it was stirred at RT for 30 min upon which LCMS showed completion. It was diluted with EtOAc and washed with water (×3). It was then washed with brine and dried over Na₂SO₄. It was concentrated down and dried under high vacuum to afford the crude title compound (+) 5a as a beige foamy solid (468 mg, crude quant. yield).

¹H NMR (DMSO-d6, 500 MHz) δ 10.43 (s, 1H), 8.50 (d, 1H, J=2.0 Hz), 8.42 (dd, 1H, J=1.3, 4.8 Hz), 7.6-7.6 (m, 1H), 7.55 (s, 1H), 7.32 (dd, 1H, J=4.8, 7.8 Hz), 7.2-7.2 (m, 2H), 6.97 (t, 1H, J=5.6 Hz), 4.20 (br d, 2H, J=5.5 Hz), 4.00 (br d, 2H, J=12.7 Hz), 3.28 (br s, 3H), 2.65 (br t, 2H, J=11.8 Hz), 2.4-2.4 (m, 6H), 2.10 (d, 1H, J=4.0 Hz), 1.69 (br d, 2H, J=11.2 Hz), 1.39 (s, 9H), 1.25 (br dd, 4H, J=3.7, 11.5 Hz), LCMS [M+H]+ 584.

### N-(2-Fluoro-4-((1S,2S)-2-(pyridin-3-yl)cyclopropane-1-carboxamido-3,3-d2)benzyl)-4-(4-(3-((5-nitropyridin-2-yl)disulfaneyl)propanoyl)piperazin-1-yl)piperidine-1-carboxamide (+) 5b

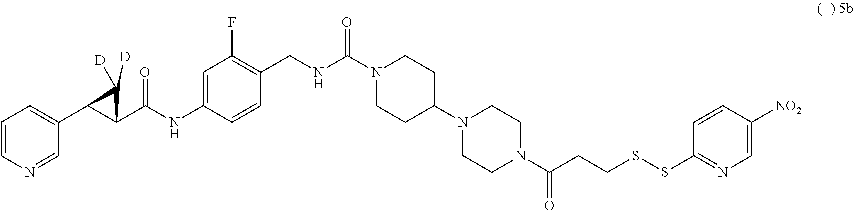 [SMILES: [2H]C1([2H])[C@H](C(=O)Nc2ccc(CNC(=O)N3CCC(N4CCN(C(=O)CCSSc5ccc([N+](=O)[O-])cn5)CC4)CC3)c(F)c2)[C@H]1c1cccnc1]

To a 250 mL RB flask containing tert-butyl 4-(1-((2-fluoro-4-((1 S,2S)-2-(pyridin-3-yl)cyclopropane-1-carboxamido-3,3-d2)benzyl)carbamoyl)piperidin-4-yl)piperazine-1-carboxylate (+) 5a (467 mg, crude) was added DCM (3 mL) and it was stirred. Trifluoroacetic acid (3 mL) was added then the mixture was stirred at RT. After about 40 min, an additional 9 mL of TFA was added. After 20 min at RT, LCMS showed complete conversion. The volatiles were evaporated off. The residue was co-evaporated with toluene (×3) then with MeOH to remove the remaining traces of TFA. The mixture was dried overnight under high vacuum. It was taken in a small amount of MeOH and passed through a PorPak column (60 cc). It was eluted with MeOH then with 3% (NH₄OH/MeOH). It was concentrated and dried under vacuum to afford the product as a light tan powder (310 mg). To this crude compound was added HATU (364 mg, 0.957 mmol) and a solution of 3-((5-nitropyridin-2-yl)disulfanyl)propanoic acid (166 mg, 0.638 mmol) in DMF (10 mL). The mixture was stirred at RT for 10 min upon which N,N-diisopropylethylamine (0.444 mL, 2.55 mmol) was added. After a further stirring of 30 min, LCMS showed completion. The mixture was diluted with EtOAc and washed with water (×4) then brine. It was dried over Na₂SO₄ and concentrated down. The residue was dried further under high vacuum to afford the crude title compound (+) 5b as a rust colored powder (376 mg, 81% yield). ¹H NMR (DMSO-d6, 500 MHz) δ 10.43 (s, 1H), 9.26 (d, 1H, J=2.3 Hz), 8.59 (dd, 1H, J=2.7, 8.9 Hz), 8.50 (d, 1H, J=2.1 Hz), 8.41 (dd, 1H, J=1.5, 4.7 Hz), 8.04 (d, 1H, J=8.9 Hz), 7.57 (br d, 1H, J=2.2 Hz), 7.55 (s, 1H), 7.32 (dd, 1H, J=4.7, 7.9 Hz), 7.2-7.2 (m, 2H), 6.97 (t, 1H, J=5.6 Hz), 4.20 (br d, 2H, J=5.5 Hz), 4.00 (br d, 2H, J=13.0 Hz), 3.09 (t, 2H, J=6.5 Hz), 2.76 (t, 2H, J=6.5 Hz), 2.65 (br t, 2H, J=11.8 Hz), 2.42 (br d, 5H, J=4.0 Hz), 2.09 (d, 1H, J=4.2 Hz), 1.68 (br d, 2H, J=11.1 Hz), LCMS [M+H]⁺ 726.

Linker-drug construct (+)-Ib

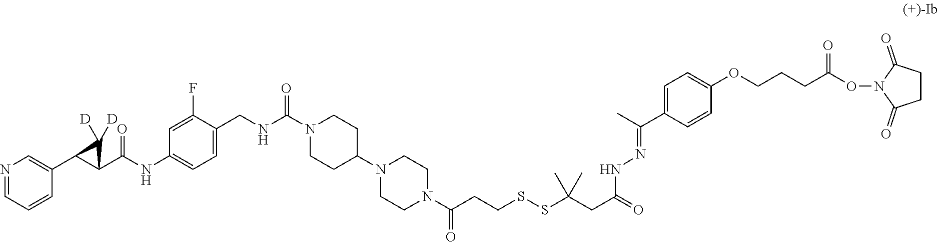 [SMILES: [2H]C1([2H])[C@H](C(=O)Nc2ccc(CNC(=O)N3CCC(N4CCN(C(=O)CCSSC(C)(C)CC(=O)N/N=C(\C)c5ccc(OCCCC(=O)ON6C(=O)CCC6=O)cc5)CC4)CC3)c(F)c2)[C@H]1c1cccnc1]

N-(2-Fluoro-4-((1S,2S)-2-(pyridin-3-yl)cyclopropane-1-carboxamido-3,3-d2)benzyl)-4-(4-(3-((5-nitropyridin-2-yl)disulfanyl)propanoyl)piperazin-1-yl)piperidine-1-carboxamide (+) 5b (27.6 mg, 0.038 mmol) was dissolved in DMF (1 mL) then 2,5-dioxopyrrolidin-1-yl-4-(4-(1-(2-(3-mercapto-3-methylbutanoyl)hydrazono)ethyl)phenoxy)butanoate (34.2 mg, 0.076 mmol) in THE (3.10 ml) was added followed by 4-methylmorpholine (0.076 mL, 0.038 mmol) as a 0.5 M solution in DMF. The mixture was stirred at room temperature for 10 min upon which LCMS showed complete conversion. The crude mixture was separated between water and EtOAc then shaken. The organic layer was washed with water (×3) then brine. It was dried over Na₂SO₄ and concentrated down. The crude was purified using CombiFlash^{RF} (4 g Gold silica column; eluent: EtOAc/hexanes; 0-100% then 100% EtOAc followed by acetone/EtOAc 0-100% then 100%). The product was taken into acetonitrile frozen then lyophilized. The title compound (+)-Ib was collected as a white fluffy powder (24 mg, 58.8% yield, 2 isomers). ¹H NMR (DMSO-d6, 500 MHz) δ 10.42 (s, 2H), 10.27 (s, 1H), 8.50 (d, 1H, J=2.1 Hz), 8.42 (dd, 1H, J=1.5, 4.7 Hz), 7.75 (dd, 2H, J=8.8, 13.2 Hz), 7.5-7.6 (m, 2H), 7.32 (dd, 1H, J=4.8, 7.9 Hz), 7.2-7.2 (m, 2H), 6.9-7.0 (m, 3H), 4.20 (br d, 2H, J=5.5 Hz), 4.10 (dt, 2H, J=2.6, 6.2 Hz), 4.00 (br d, 2H, J=13.8 Hz), 3.41 (br s, 3H), 2.95 (br t, 2H, J=6.7 Hz), 2.86 (td, 3H, J=3.6, 7.2 Hz), 2.82 (br s, 4H), 2.7-2.7 (m, 2H), 2.64 (s, 3H), 2.60 (s, 1H), 2.42 (br d, 3H, J=4.2 Hz), 2.37 (br d, 1H, J=2.0 Hz), 2.23 (s, 1H), 2.21 (s, 2H), 2.12 (s, 1H), 2.09 (br s, 2H), 2.08 (s, 1H), 1.67 (br t, 2H, J=8.5 Hz), 1.42 (s, 6H), 1.24 (br s, 3H); LCMS [M+H]⁺ 1019.

Linker-drug construct (+) Ip

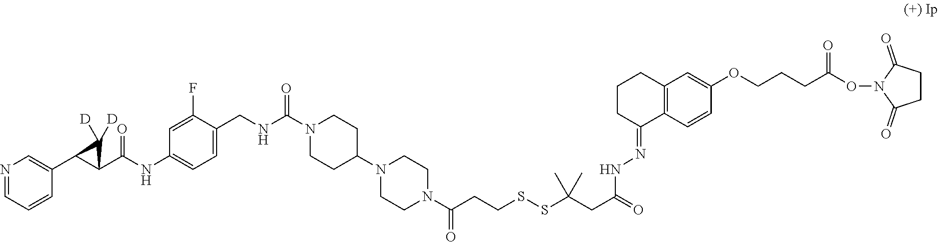 [SMILES: [2H]C1([2H])[C@H](C(=O)Nc2ccc(CNC(=O)N3CCC(N4CCN(C(=O)CCSSC(C)(C)CC(=O)N/N=C5\CCCc6cc(OCCCC(=O)ON7C(=O)CCC7=O)ccc65)CC4)CC3)c(F)c2)[C@H]1c1cccnc1]

The title compound (+) Ip was prepared using similar procedures to (+)-Ib. It was collected as an off-white fluffy powder (19.3 mg, 49.3% yield, 2 isomers). ¹H NMR (DMSO-d6, 500 MHz) δ 10.42 (s, 1H), 10.35 (s, 1H), 10.20 (s, 1H), 8.50 (d, 1H, J=1.6 Hz), 8.42 (dd, 1H, J=1.2, 4.7 Hz), 7.9-8.0 (m, 1H), 7.57 (br d, 1H, J=5.7 Hz), 7.55 (s, 1H), 7.32 (dd, 1H, J=4.8, 7.8 Hz), 7.2-7.2 (m, 2H), 6.96 (br s, 1H), 6.86 (ddd, 1H, J=2.4, 8.7, 17.5 Hz), 6.77 (s, 1H), 4.20 (br d, 2H, J=5.5 Hz), 4.1-4.1 (m, 2H), 4.00 (br d, 2H, J=12.6 Hz), 3.41 (br s, 2H), 3.07 (s, 1H), 2.95 (q, 2H, J=6.6 Hz), 2.8-2.9 (m, 2H), 2.82 (br s, 4H), 2.7-2.7 (m, 5H), 2.6-2.7 (m, 3H), 2.60 (s, 2H), 2.4-2.4 (m, 4H), 2.37 (br d, 2H, J=1.8 Hz), 2.1-2.1 (m, 3H), 1.8-1.8 (m, 2H), 1.67 (br d, 2H, J=8.2 Hz), 1.42 (s, 6H), 1.24 (br s, 5H), 1.15 (s, 1H); LCMS [M+H]+ 10449.

Linker-drug construct (+) Id

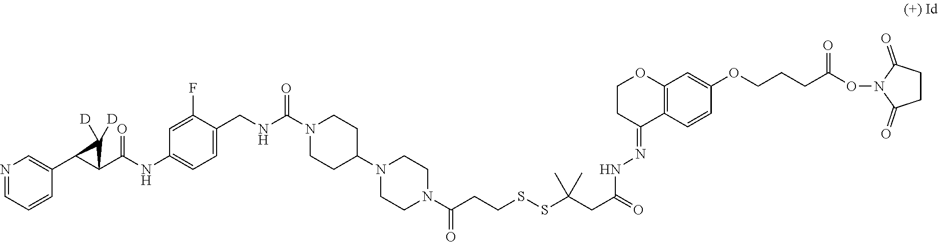 [SMILES: [2H]C1([2H])[C@H](C(=O)Nc2ccc(CNC(=O)N3CCC(N4CCN(C(=O)CCSSC(C)(C)CC(=O)N/N=C5\CCOc6cc(OCCCC(=O)ON7C(=O)CCC7=O)ccc65)CC4)CC3)c(F)c2)[C@H]1c1cccnc1]

The title compound (+) Id was prepared using similar procedures to (+)-Ib. It was collected as a white fluffy powder (26.5 mg, 64.8% yield, 2 isomers). ¹H NMR (DMSO-d6, 500 MHz) δ 10.49 (s, 1H), 10.42 (s, 1H), 10.33 (s, 1H), 8.50 (d, 1H, J=2.1 Hz), 8.42 (dd, 1H, J=1.5, 4.7 Hz), 7.8-7.9 (m, 1H), 7.57 (br dd, 1H, J=2.6, 4.7 Hz), 7.55 (s, 1H), 7.32 (dd, 1H, J=4.8, 7.8 Hz), 7.2-7.2 (m, 2H), 6.96 (br d, 1H, J=2.2 Hz), 6.64 (ddd, 1H, J=2.5, 8.8, 16.9 Hz), 6.47 (d, 1H, J=1.2 Hz), 4.2-4.3 (m, 5H), 4.0-4.1 (m, 2H), 4.00 (br d, 2H, J=12.2 Hz), 3.41 (br s, 2H), 3.06 (s, 1H), 2.9-3.0 (m, 2H), 2.85 (br s, 1H), 2.82 (br s, 4H), 2.8-2.8 (m, 2H), 2.69 (br t, 2H, J=7.0 Hz), 2.6-2.7 (m, 2H), 2.61 (br s, 1H), 2.60 (s, 1H), 2.3-2.4 (m, 7H), 2.0-2.1 (m, 4H), 1.89 (s, 1H), 1.68 (br s, 2H), 1.43 (s, 3H), 1.42 (br s, 3H), 1.24 (br s, 4H), 1.15 (s, 1H); LCMS [M+H]+ 1047.

Linker-drug construct (+) If

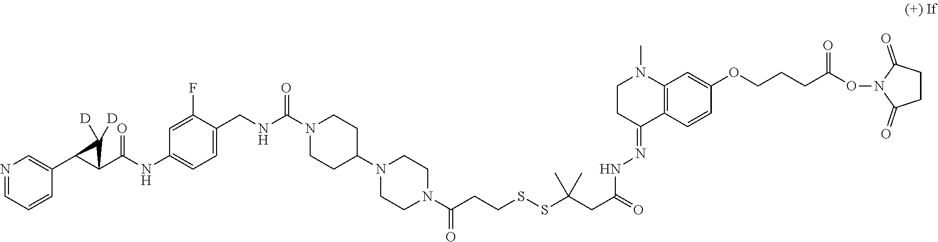 [SMILES: [2H]C1([2H])[C@H](C(=O)Nc2ccc(CNC(=O)N3CCC(N4CCN(C(=O)CCSSC(C)(C)CC(=O)N/N=C5\CCN(C)c6cc(OCCCC(=O)ON7C(=O)CCC7=O)ccc65)CC4)CC3)c(F)c2)[C@H]1c1cccnc1]

The title compound (+) If was prepared using similar procedures to (+)-Ib. It was collected as a light yellow fluffy powder (22.3 mg, 51.7% yield, 2 isomers). ¹H NMR (DMSO-d6, 500 MHz) δ 10.47 (s, 1H), 10.34 (s, 1H), 10.22 (s, 1H), 8.56 (d, 1H, J=1.8 Hz), 8.47 (dd, 1H, J=1.3, 4.6 Hz), 7.9-7.9 (m, 1H), 7.6-7.6 (m, 1H), 7.61 (s, 1H), 7.38 (dd, 1H, J=4.8, 7.8 Hz), 7.2-7.3 (m, 2H), 7.02 (br d, 1H, J=2.8 Hz), 6.44 (ddd, 1H, J=2.3, 8.7, 18.2 Hz), 6.31 (s, 1H), 4.26 (br d, 2H, J=5.6 Hz), 4.1-4.2 (m, 2H), 4.05 (br d, 2H, J=11.1 Hz), 3.46 (br s, 2H), 3.2-3.2 (m, 3H), 3.10 (s, 1H), 3.00 (q, 2H, J=6.8 Hz), 2.90 (br d, 5H, J=8.6 Hz), 2.88 (br s, 4H), 2.7-2.8 (m, 5H), 2.7-2.7 (m, 2H), 2.6-2.7 (m, 2H), 2.4-2.5 (m, 3H), 2.42 (br t, 2H, J=1.8 Hz), 2.1-2.2 (m, 3H), 1.7-1.8 (m, 2H), 1.47 (s, 6H), 1.30 (s, 4H); [M+H]⁺ 1060.

Linker-drug construct (+) Is

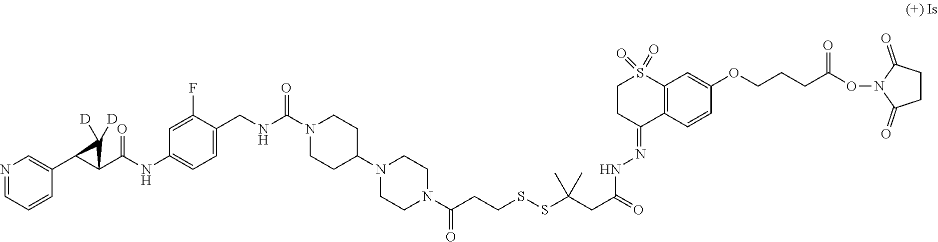 [SMILES: [2H]C1([2H])[C@H](C(=O)Nc2ccc(CNC(=O)N3CCC(N4CCN(C(=O)CCSSC(C)(C)CC(=O)N/N=C5\CCS(=O)(=O)c6cc(OCCCC(=O)ON7C(=O)CCC7=O)ccc65)CC4)CC3)c(F)c2)[C@H]1c1cccnc1]

The title compound (+) Is was prepared using similar procedures to (+)-Ib. It was collected as a white fluffy powder (25.3 mg, 61.6% yield, 2 isomers). ¹H NMR (DMSO-d6, 500 MHz) δ 10.83 (s, 1H), 10.59 (s, 1H), 10.42 (s, 1H), 8.50 (d, 1H, J=2.0 Hz), 8.42 (dd, 1H, J=1.5, 4.6 Hz), 8.1-8.2 (m, 1H), 7.6-7.6 (m, 1H), 7.55 (s, 1H), 7.39 (dd, 1H, J=2.6, 9.0 Hz), 7.32 (dd, 2H, J=4.9, 7.8 Hz), 7.29 (d, 1H, J=2.6 Hz), 7.2-7.2 (m, 2H), 6.96 (br d, 1H, J=2.3 Hz), 4.2-4.2 (m, 4H), 4.00 (br d, 2H, J=12.5 Hz), 3.7-3.8 (m, 2H), 3.41 (br s, 2H), 3.2-3.2 (m, 3H), 3.10 (s, 1H), 2.96 (q, 2H, J=6.5 Hz), 2.88 (dt, 2H, J=3.4, 7.1 Hz), 2.82 (br s, 4H), 2.7-2.7 (m, 3H), 2.64 (br d, 2H, J=1.6 Hz), 2.4-2.4 (m, 5H), 2.37 (dd, 2H, J=1.8, 3.5 Hz), 2.1-2.1 (m, 4H), 1.7-1.7 (m, 2H), 1.43 (s, 6H), 1.24 (br s, 4H), 1.15 (s, 1H); LCMS [M+H]+ 1095.

### Linker-Drug Construct (+) Icc

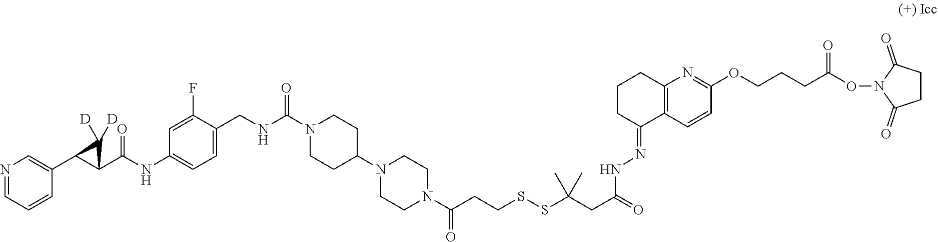 [SMILES: [2H]C1([2H])[C@H](C(=O)Nc2ccc(CNC(=O)N3CCC(N4CCN(C(=O)CCSSC(C)(C)CC(=O)N/N=C5\CCCc6nc(OCCCC(=O)ON7C(=O)CCC7=O)ccc65)CC4)CC3)c(F)c2)[C@H]1c1cccnc1]

The title compound (+) Icc was prepared using similar procedures to (+)-Ib. It was collected as a white fluffy powder (9.3 mg, 22.2% yield, 2 isomers). ¹H NMR (DMSO-d6, 500 MHz) δ 10.53 (s, 1H), 10.47 (s, 1H), 10.38 (s, 1H), 8.55 (d, 1H, J=2.0 Hz), 8.47 (dd, 1H, J=1.4, 4.7 Hz), 8.2-8.3 (m, 1H), 7.6-7.6 (m, 1H), 7.60 (s, 1H), 7.37 (dd, 1H, J=4.7, 7.9 Hz), 7.2-7.3 (m, 2H), 7.01 (br s, 1H), 6.81 (dd, 1H, J=8.7, 19.9 Hz), 4.4-4.4 (m, 2H), 4.25 (br d, 2H, J=5.5 Hz), 4.05 (br d, 2H, J=12.2 Hz), 3.46 (br s, 4H), 3.35 (s, 4H), 3.11 (s, 1H), 3.00 (br t, 2H, J=6.8 Hz), 2.90 (br d, 2H, J=3.1 Hz), 2.87 (br s, 4H), 2.8-2.9 (m, 2H), 2.7-2.8 (m, 3H), 2.7-2.7 (m, 3H), 2.6-2.7 (m, 3H), 2.4-2.5 (m, 6H), 2.17 (s, 1H), 2.1-2.2 (m, 3H), 1.9-2.0 (m, 2H), 1.73 (br s, 2H), 1.47 (s, 6H), 1.29 (br s, 4H), 1.20 (s, 2H); LCMS [M+H]+ 1046.

Linker-drug construct (+) Ix

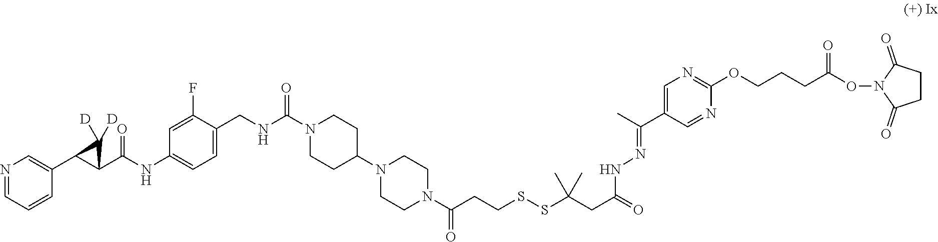 [SMILES: [2H]C1([2H])[C@H](C(=O)Nc2ccc(CNC(=O)N3CCC(N4CCN(C(=O)CCSSC(C)(C)CC(=O)N/N=C(\C)c5cnc(OCCCC(=O)ON6C(=O)CCC6=O)nc5)CC4)CC3)c(F)c2)[C@H]1c1cccnc1]

The title compound (+) Ix was prepared using similar procedures to (+)-Ib. It was collected as an off-white fluffy powder (21.6 mg, 52.3% yield, 2 isomers). ¹H NMR (DMSO-d6, 500 MHz) δ 10.66 (s, 1H), 10.49 (s, 1H), 10.42 (s, 1H), 8.98 (s, 1H), 8.92 (s, 1H), 8.50 (d, 1H, J=2.0 Hz), 8.42 (dd, 1H, J=1.4, 4.7 Hz), 7.6-7.6 (m, 1H), 7.55 (s, 1H), 7.32 (dd, 1H, J=4.8, 7.9 Hz), 7.2-7.2 (m, 2H), 6.9-7.0 (m, 1H), 4.42 (t, 2H, J=6.4 Hz), 4.20 (br d, 2H, J=5.6 Hz), 4.00 (br d, 2H, J=12.2 Hz), 3.4-3.4 (m, 4H), 3.30 (s, 4H), 3.06 (s, 1H), 2.94 (td, 2H, J=6.9, 10.9 Hz), 2.86 (dt, 2H, J=1.9, 7.3 Hz), 2.82 (s, 4H), 2.7-2.7 (m, 1H), 2.67 (s, 2H), 2.6-2.7 (m, 2H), 2.4-2.4 (m, 6H), 2.27 (d, 3H, J=11.7 Hz), 2.1-2.1 (m, 4H), 1.68 (br d, 2H, J=11.5 Hz), 1.43 (br s, 3H), 1.42 (br s, 3H), 1.24 (s, 3H), 1.15 (s, 3H); LCMS [M+H]⁺ 1021.

Linker-drug construct (+) Ibb

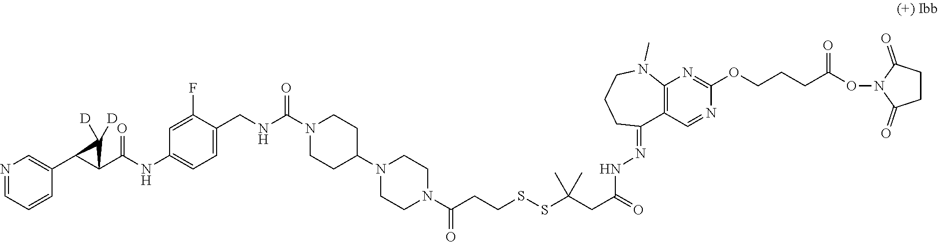 [SMILES: [2H]C1([2H])[C@H](C(=O)Nc2ccc(CNC(=O)N3CCC(N4CCN(C(=O)CCSSC(C)(C)CC(=O)N/N=C5\CCCN(C)c6nc(OCCCC(=O)ON7C(=O)CCC7=O)ncc65)CC4)CC3)c(F)c2)[C@H]1c1cccnc1]

The title compound (+) Ibb was prepared using similar procedures to (+)-Ib. It was collected as a beige fluffy powder (18.9 mg, 49.1% yield, 2 isomers). ¹H NMR (DMSO-d6, 500 MHz) δ 10.4-10.4 (m, 1H), 10.34 (s, 1H), 10.11 (s, 1H), 8.43 (d, 1H, J=1.8 Hz), 8.34 (dd, 1H, J=1.3, 4.8 Hz), 7.5-7.5 (m, 1H), 7.48 (s, 1H), 7.3-7.4 (m, 1H), 7.25 (dd, 1H, J=4.8, 7.9 Hz), 7.1-7.2 (m, 2H), 6.8-6.9 (m, 1H), 6.3-6.4 (m, 1H), 6.30 (d, 1H, J=2.2 Hz), 4.13 (br d, 2H, J=5.5 Hz), 4.0-4.0 (m, 2H), 3.93 (br d, 2H, J=12.0 Hz), 3.34 (br s, 3H), 3.23 (s, 4H), 2.9-3.0 (m, 3H), 2.86 (q, 2H, J=6.9 Hz), 2.8-2.8 (m, 5H), 2.75 (br s, 4H), 2.6-2.6 (m, 5H), 2.55 (br s, 2H), 2.3-2.4 (m, 6H), 2.05 (s, 1H), 2.0-2.0 (m, 3H), 1.8-1.8 (m, 2H), 1.61 (br s, 2H), 1.35 (br s, 3H), 1.34 (br s, 3H), 1.17 (s, 5H), 1.07 (s, 3H); LCMS [M+H]⁺ 1074.

### Conjugation of NAMPTI-Linker Construct of Formula I to Antibodies

In some embodiments, the linker-drug conjugate of Formula I is chemically conjugated to accessible lysine residues on antibodies. For example, as shown in Schemes 6 and 7, exemplary drug, NAMPT inhibitor, is chemically linked to surface accessible lysine residues on human IgG1 antibodies such as Trastuzumab or Cetuximab by reaction of linker-drug conjugates of Formula (I) with the respective antibody to provide the ADCs of Formula IV.

### Conjugation of Linker-Drug Conjugates of Formula (I) to Cetuximab

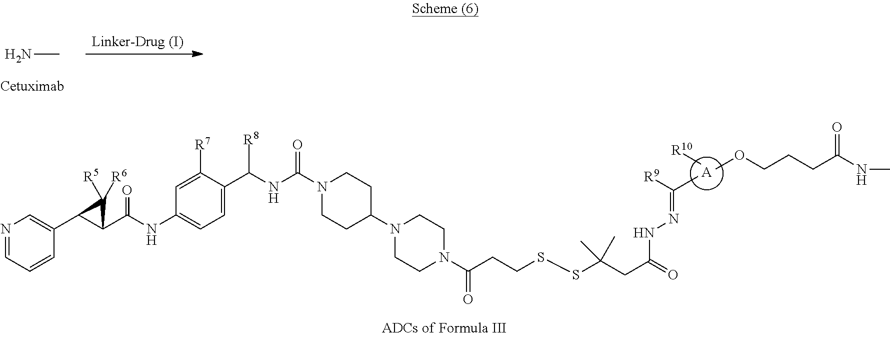

### Conjugation of Linker-Drug Conjugates of Formula (I) to Trastuzumab

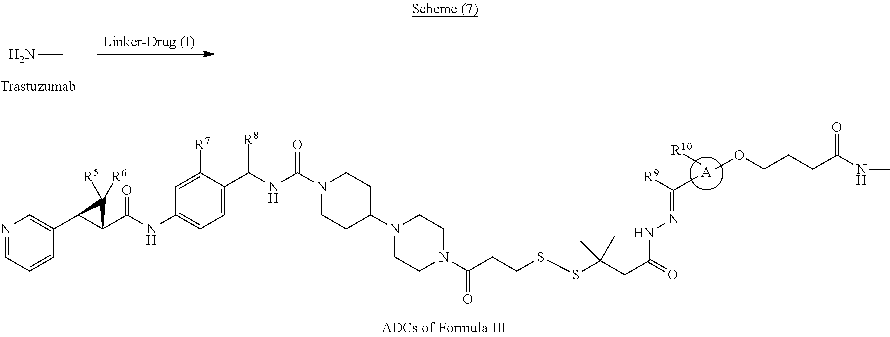

In an exemplary embodiment, the NAMPTi payload was chemically linked to surface accessible lysine residues on the human IgG1 antibody Trastuzumab by reaction of drug-linker constructs (I) with the antibody.

### Synthesis and Analysis of ADCs

220 ug of Trastuzumab (final concentration of 2 mg/mL) in 1×conjugation buffer (100 mM sodium phosphate, 20 mM sodium chloride, 2 mM EDTA pH7.4) with 20% v/v DMA cosolvent final concentration was incubated with different linker-drugs with a NAMPTi payload for 2 hours at 32° C.

An automated buffer exchange to remove DMA and unincorporated linker-drug was performed by the Hamilton Star liquid handler. Samples were passed once through IMCS size×150 resin-filled tips pre-equilibrated with formulation buffer (20 mM sodium phosphate pH 7.4 0.02% Tween-20). Following buffer exchange, samples were centrifuged for 10 minutes at 20,000×g (4° C.).

Drug: Antibody ratio (DAR) and protein concentrations were determined by absorbance readings at 280 nm and 257 nm. Monomeric purity was determined by HPLC Size-Exclusion Chromatography. The DAR and monomeric purity measurements are shown in Table 2.

TABLE 1
Linker-drug extinction coefficient and ratio to Trastuzumab.

| | Extinction | Extinction | Linker- |
|---|---|---|---|
| Linker- | coefficient | coefficient | drug/Trastuzumab |
| drug | @280 nm | @257 nm | ratio |
| Ib | 22848 | 28584 | 10x |
| Id | 9400 | 11600 | 10x |
| If | 40155 | 37286 | 8x |
| (+) Ib | 28096 | 24714 | 10x |
| (+) Id | 8212 | 8400 | 6x |
| (+) If | 19761 | 17054 | 6x |
| (+) Ip | 30371 | 29481 | 10x |
| (+) Is | 2400 | 7798 | 10x |
| (+) Icc | 24096 | 21146 | 8x |
| (+) Ix | 6600 | 7541 | 8x |

TABLE 2
Conjugation with Trastuzumab results

| Linker- | Trastuzumab | | Monomeric purity by |
|---|---|---|---|
| drug | ADC | DAR | HPLC-SEC (%) |
| Ib | IIIb | 2.14 | 99.73 |
| Id | IIId | 5.39 | 100.00 |
| If | IIIf | 3.71 | 100.00 |
| (+) Ib | (+) IIIb | 1.17 | 93.89 |
| (+) Id | (+) IIId | 4.81 | 99.74 |
| (+) If | (+) IIIf | 4.37 | 90.77 |
| (+) Ip | (+) IIIp | 2.10 | 100.00 |
| (+) Is | (+) IIIs | 4.73 | 100.00 |
| (+) Icc | (+) IIIcc | 5.20 | 99.21 |
| (+) Ix | (+) IIIx | 10.47 | 98.72 |

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE APPLICATION

(1) a. In American Cancer Society. “Chemotherapy side effects” 2017. Available at https://www.cancer.org/treatment/treatments-and-side-effects/treatment-types/chemotherapy/chemotherapy-side-effects.html (Accessed Sep. 14, 2017);
b. Beck, A.; Goetsch L.; Dumontet, C. and Corvaia, N. Strategies and challenges of next generation antibody-drug conjugates, Nat. Rev. Drug. Discov. 2017, 16, 315-337.
(2) Chabner, B. A.; Roberts, T. G., Jr. Timeline: Chemotherapy and the war on cancer, Nat. Rev. Cancer 2005, 5, 65-72.
(3) Allen, T. M. Ligand-targeted therapeutics in anticancer therapy, Nat. Rev. Cancer 2002, 2, 750-763.
(4) Helleday, T.; Petermann, E.; Lundin, C.; Hodgson, B.; Sharma, R. A. DNA repair pathways as targets for cancer therapy, Nat. Rev. Cancer 2008, 8, 193-204.
(5) Zhang, J.; Yang, P. L.; Gray, N. S. Targeting cancer with small molecule kinase inhibitors, Nat. Rev. Cancer 2009, 9, 28-39.
(6) Aggarwal, S. Targeted cancer therapies, Nat. Rev. Drug. Discov. 2010, 9, 427-428.
(7) Tennant, D. A.; Duran, R. V.; Gottlieb, E. Targeting metabolic transformation for cancer therapy, Nat. Rev. Cancer 2010, 10, 267-277.
(8) Imai, K.; Takaoka, A. Comparing antibody and small-molecule therapies for cancer, Nat. Rev. Cancer 2006, 6, 714-727.
(9) Weiner, L. M.; Surana, R.; Wang, S. Monoclonal antibodies: versatile platforms for cancer immunotherapy, Nat. Rev. Immunol. 2010, 10, 317-327.
(10) Reichert, J. M. Antibody-based therapeutics to watch in 2011, MAbs 2011, 3, 76-99.
(11) Murphy, K. P.; Travers, P.; Walport, M.; Janeway, C. Janeway's Immunobiology; Garland Science: New York, 2008.
(12) Trail, P. A.; Willner, D.; Lasch, S. J.; Henderson, A. J.; Hofstead, S.; Casazza, A. M.; Firestone, R. A.; Hellstrom, I.; Hellstrom, K. E. Cure of xenografted human carcinomas by BR96-doxorubicin immunoconjugates, Science 1993, 261, 212-215.
(13) Alley, S. C.; Okeley, N. M.; Senter, P. D. Antibody-drug conjugates: targeted drug delivery for cancer, Curr. Opin. Chem. Biol. 2010, 14, 529-537.
(14) Ducry, L.; Stump, B. Antibody-drug conjugates: linking cytotoxic payloads to monoclonal antibodies, Bioconjug. Chem. 2010, 21, 5-13.
(15) Casi, G.; Neri, D. Antibody-drug conjugates: basic concepts, examples and future perspectives, J. Control. Release 2012, 161, 422-428.
(16) Adair, J. R.; Howard, P. W.; Hartley, J. A.; Williams, D. G.; Chester, K. A. Antibody-drug conjugates—a perfect synergy, Expert Opin. Biol. Ther. 2012, 12, 1191-1206.
(17) Carter, P. J. Potent antibody therapeutics by design, Nat. Rev. Immunol. 2006, 6, 343-357.
(18) Teicher, B. A. Antibody-drug conjugate targets, Curr. Cancer Drug Targets 2009, 9, 982-1004.
(19) Doronina, S. O.; Toki, B. E.; Torgov, M. Y.; Mendelsohn, B. A.; Cerveny, C. G.; Chace, D. F.; DeBlanc, R. L.; Gearing, R. P.; Bovee, T. D.; Siegall, C. B.; Francisco, J. A.; Wahl, A. F.; Meyer, D. L.; Senter, P. D. Development of potent monoclonal antibody auristatin conjugates for cancer therapy, Nat. Biotechnol. 2003, 21, 778-784.
(20) Widdison, W. C.; Wilhelm, S. D.; Cavanagh, E. E.; Whiteman, K. R.; Leece, B. A.; Kovtun, Y.; Goldmacher, V. S.; Xie, H.; Steeves, R. M.; Lutz, R. J.; Zhao, R.; Wang, L.; Blattler, W. A.; Chari, R. V. Semisynthetic maytansine analogues for the targeted treatment of cancer, J. Med. Chem. 2006, 49, 4392-4408.
(21) Hartley, J. A. The development of pyrrolobenzodiazepines as antitumour agents, Expert Opin. Investig. Drugs 2011, 20, 733-744.
(22) Doronina, S. O.; Mendelsohn, B. A.; Bovee, T. D.; Cerveny, C. G.; Alley, S. C.; Meyer, D. L.; Oflazoglu, E.; Toki, B. E.; Sanderson, R. J.; Zabinski, R. F.; Wahl, A. F.; Senter, P. D. Enhanced activity of monomethylauristatin F through monoclonal antibody delivery: effects of linker technology on efficacy and toxicity, Bioconjug. Chem. 2006, 17, 114-124.
(23) Dosio, F.; Brusa, P.; Cattel, L. Immunotoxins and Anticancer Drug Conjugate Assemblies: The Role of the Linkage between Components, Toxins (Basel) 2011, 3, 848-883.
(24) Wu, A. M.; Senter, P. D. Arming antibodies: prospects and challenges for immunoconjugates, Nat. Biotechnol. 2005, 23, 1137-1146.
(25) Ansell, S. M. Brentuximab vedotin: delivering an antimitotic drug to activated lymphoma cells, Expert Opin. Investig. Drugs 2011, 20, 99-105.
(26) Katz, J.; Janik, J. E.; Younes, A. Brentuximab Vedotin (SGN-35), Clin. Cancer Res. 2011, 17, 6428-6436.
(27) Hamann, P. R.; Hinman, L. M.; Hollander, I.; Beyer, C. F.; Lindh, D.; Holcomb, R.; Hallett, W.; Tsou, H. R.; Upeslacis, J.; Shochat, D.; Mountain, A.; Flowers, D. A.; Bernstein, I. Gemtuzumab ozogamicin, a potent and selective anti-CD33 antibody-calicheamicin conjugate for treatment of acute myeloid leukemia, Bioconjug. Chem. 2002, 13, 47-58.
(28) Hutter, M. L.; Schlenk, R. F. Gemtuzumab ozogamicin in non-acute promyelocytic acute myeloid leukemia, Expert Opin. Biol. Ther. 2011, 11, 1369-1380.
(29) Remillard, S.; Rebhun, L. I.; Howie, G. A.; Kupchan, S. M. Antimitotic activity of the potent tumor inhibitor maytansine, Science 1975, 189, 1002-1005.
(30) Goldberg, R. M. Cetuximab, Nat. Rev. Drug Discov. 2005, Suppl, S10-11.
(31) Kabolizadeh, P.; Kubicek, G. J.; Heron, D. E.; Ferris, R. L.; Gibson, M. K. The role of cetuximab in the management of head and neck cancers, Expert Opin. Biol. Ther. 2012, 12, 517-528.
(32) Broadbridge, V. T.; Karapetis, C. S.; Price, T. J. Cetuximab in metastatic colorectal cancer, Expert Rev. Anticancer Ther. 2012, 12, 555-565.
(33) Arteaga, C. L. Overview of epidermal growth factor receptor biology and its role as a therapeutic target in human neoplasia, Semin. Oncol. 2002, 29, 3-9.
(34) Mendelsohn, J.; Baselga, J. Status of epidermal growth factor receptor antagonists in the biology and treatment of cancer, J. Clin. Oncol. 2003, 21, 2787-2799.
(35) Krause, D. S.; Van Etten, R. A. Tyrosine kinases as targets for cancer therapy, N. Engl. J. Med. 2005, 353, 172-187.
(36) Goldstein, N. I.; Prewett, M.; Zuklys, K.; Rockwell, P.; Mendelsohn, J. Biological efficacy of a chimeric antibody to the epidermal growth factor receptor in a human tumor xenograft model, Clin. Cancer Res. 1995, 1, 1311-1318.
(37) Hamann, P. R.; Hinman, L. M.; Beyer, C. F.; Lindh, D.; Upeslacis, J.; Flowers, D. A.; Bernstein, I. An anti-CD33 antibody-calicheamicin conjugate for treatment of acute myeloid leukemia. Choice of linker, Bioconjug. Chem. 2002, 13, 40-46.
(38) van Der Velden, V. H.; te Marvelde, J. G.; Hoogeveen, P. G.; Bernstein, I. D.; Houtsmuller, A. B.; Berger, M. S.; van Dongen, J. J. Targeting of the CD33-calicheamicin immunoconjugate Mylotarg (CMA-676) in acute myeloid leukemia: in vivo and in vitro saturation and internalization by leukemic and normal myeloid cells, Blood 2001, 97, 3197-3204.
(39) Ojima, I.; Geng, X.; Wu, X.; Qu, C.; Borella, C. P.; Xie, H.; Wilhelm, S. D.; Leece, B. A.; Bartle, L. M.; Goldmacher, V. S.; Chari, R. V. Tumor-specific novel taxoid-monoclonal antibody conjugates, J. Med. Chem. 2002, 45, 5620-5623.
(40) Erickson, H. K.; Park, P. U.; Widdison, W. C.; Kovtun, Y. V.; Garrett, L. M.; Hoffman, K.; Lutz, R. J.; Goldmacher, V. S.; Blattler, W. A. Antibody-maytansinoid conjugates are activated in targeted cancer cells by lysosomal degradation and linker-dependent intracellular processing, Cancer Res. 2006, 66, 4426-4433.
(41) Lewis Phillips, G. D.; Li, G.; Dugger, D. L.; Crocker, L. M.; Parsons, K. L.; Mai, E.; Blattler, W. A.; Lambert, J. M.; Chari, R. V.; Lutz, R. J.; Wong, W. L.; Jacobson, F. S.; Koeppen, H.; Schwall, R. H.; Kenkare-Mitra, S. R.; Spencer, S. D.; Sliwkowski, M. X. Targeting HER2-positive breast cancer with trastuzumab-DM1, an antibody-cytotoxic drug conjugate, Cancer Res. 2008, 68, 9280-9290.
(42) Kellogg, B. A.; Garrett, L.; Kovtun, Y.; Lai, K. C.; Leece, B.; Miller, M.; Payne, G.; Steeves, R.; Whiteman, K. R.; Widdison, W.; Xie, H.; Singh, R.; Chari, R. V.; Lambert, J. M.; Lutz, R. J. Disulfide-linked antibody-maytansinoid conjugates: optimization of in vivo activity by varying the steric hindrance at carbon atoms adjacent to the disulfide linkage, Bioconjug. Chem. 2011, 22, 717-727.
(43) Dubowchik, G. M.; Mosure, K.; Knipe, J. O.; Firestone, R. A. Cathepsin B-sensitive dipeptide prodrugs. 2. Models of anticancer drugs paclitaxel (Taxol), mitomycin C and doxorubicin, Bioorg. Med. Chem. Lett. 1998, 8, 3347-3352.
(44) Francisco, J. A.; Cerveny, C. G.; Meyer, D. L.; Mixan, B. J.; Klussman, K.; Chace, D. F.; Rejniak, S. X.; Gordon, K. A.; DeBlanc, R.; Toki, B. E.; Law, C. L.; Doronina, S. O.; Siegall, C. B.; Senter, P. D.; Wahl, A. F. cAC10-vcMMAE, an anti-CD30-monomethyl auristatin E conjugate with potent and selective antitumor activity, Blood 2003, 102, 1458-1465.
(45) Sanderson, R. J.; Hering, M. A.; James, S. F.; Sun, M. M.; Doronina, S. O.; Siadak, A. W.; Senter, P. D.; Wahl, A. F. In vivo drug-linker stability of an anti-CD30 dipeptide-linked auristatin immunoconjugate, Clin. Cancer Res. 2005, 11, 843-852.
(46) DiJoseph, J. F.; Dougher, M. M.; Evans, D. Y.; Zhou, B. B.; Damle, N. K. Preclinical anti-tumor activity of antibody-targeted chemotherapy with CMC-544 (inotuzumab ozogamicin), a CD22-specific immunoconjugate of calicheamicin, compared with non-targeted combination chemotherapy with CVP or CHOP, Cancer Chemother. Pharmacol. 2011, 67, 741-749.
(47) Toki, B. E.; Cerveny, C. G.; Wahl, A. F.; Senter, P. D. Protease-mediated fragmentation of p-amidobenzyl ethers: a new strategy for the activation of anticancer prodrugs, J. Org. Chem. 2002, 67, 1866-1872.
(48) Kovtun, Y. V.; Audette, C. A.; Ye, Y.; Xie, H.; Ruberti, M. F.; Phinney, S. J.; Leece, B. A.; Chittenden, T.; Blattler, W. A.; Goldmacher, V. S. Antibody-drug conjugates designed to eradicate tumors with homogeneous and heterogeneous expression of the target antigen, Cancer Res. 2006, 66, 3214-3221.
(49) Ogitani, Y.; Hagihara, K.; Oitate, M.; Naito, H.; Agatsuma T. Bystander killing effect of DS-8201a, a novel anti-human epidermal growth factor receptor 2 antibody-drug conjugate, in tumors with human epidermal growth factor receptor 2 heterogeneity. Cancer Sci. 2016, 107, 1039.
(50) Erickson, H. K.; Park, P. U.; Widdison, W. C.; Kovtun, Y. V.; Garrett, L. M.; Hoffman, K.; Lutz, R. J.; Goldmacher, V. S.; Blsttler, W. A. Antibody-Maytansinoid Conjugates Are Activated in Targeted Cancer Cells by Lysosomal Degradation and Linker-Dependent Intracellular Processing. Cancer Res. 2006, 66, 4426.
(51) Elgersma, R. C.; Coumans, R. G. E; Huijbregts, T.; Menge, W. M. P. B.; Joosten, J. A. F; Spijker, H. J.; de Groot, F. M. H.; van der Lee, M. M. C.; Ubink, R.; van den Dobbelsteen, D. J.; Egging, D. F.; Dokter, W. H. A; Verheijden, G. F. M.; Lemmens, J. M.; C. Marco Timmers, C. M.; Beusker, P. H. Design, Synthesis, and Evaluation of Linker-Duocarmycin Payloads: Toward Selection of HER2-Targeting Antibody-Drug Conjugate SYD985. Mol. Pharmaceutics, 2015, 126, 1813.
(52) Jackson, P. J. M.; Kay, S.; Pysz, I.; Thurston, D. E. Use of pyrrolobenzodiazepines and related covalent-binding DNA-interactive molecules as ADC payloads: Is mechanism related to systemic toxicity?Drug Discov. Today Technol. 2018, 30, 71.
(53) Lerchen, H. G.; Wittrock, S.; Stelte-Ludwig, B.; Sommer, A.; Berndt, S.; Griebenow, N.; Rebstock, A. S.; Johannes, S.; Cancho-Grande, Y.; Mahlert, C.; Greven, S.; Terjung C. Antibody-Drug Conjugates with Pyrrole-Based KSP Inhibitors as the Payload Class. Angew. Chem. Int. Ed. Engl. 2018, 57, 15243.
(54) Karpov, A. S.; Tinya Abrams, T.; Clark, S.; Raikar, A.; D'Alessio, J. A.; Dillon, M. P.; Gesner, T. G.; Jones, D.; Lacaud, M.; Mallet, W.; Martyniuk, P.; Meredith, E.; Mohseni, M.; Nieto-Oberhuber, C. M.; Palacios, D.; Perruccio, F.; Piizzi, G.; Zurini, M.; Bialucha, C. U. Nicotinamide Phosphoribosyltransferase Inhibitor as a Novel Payload for Antibody-Drug Conjugates. ACS. Med. Chem. Lett. 2018, 9, 838.
(55) Neumann, C. S.; Olivas, K. C.; Anderson, M. E.; Cochran, J. H.; Jin, S.; Li, F.; Loftus, L. V.; Meyer, D. W.; Neale, J.; Nix, J. C.; Pittman, P. G.; Simmons, J. K.; Ulrich, M. L.; Waight, A. B.; Wong, A.; Zaval, M. C.; Zeng, W.; Lyon, R. P.; Senter, P. D. Targeted Delivery of Cytotoxic NAMPT Inhibitors Using Antibody-Drug Conjugates. Mol. Cancer Ther. 2018, 17(12); 2633.
(56) Roulston, A.; Shore, G. C. New Strategies to Maximize Therapeutic Opportunities for NAMPT Inhibitors in Oncology. Molecular & Cellular Oncology. 2016, 3, e1052180.
(57) Canto, C.; Menzies Keirm J.; Auwerx, J. NAD+ Metabolism and the control of energy homeostasis: a balancing act between mitochondria and the nucleus. Cell Metabolism. 2015, 22, 31.
(58) Del Nagro, C; Xiao, Y.; Rangell, L.; Reichelt, M.; O'Brien, T. Depletion of the central metabolite NAD leads to oncosis-mediated cell death. J. Biol. Chem. 2014, 289, 35182.
(59) Gant, T. G. Using Deuterium in Drug Discovery; Leaving the Label in the Drug. J. Med. Chem. 2014, 57, 3595

1. A compound of Formula (I): 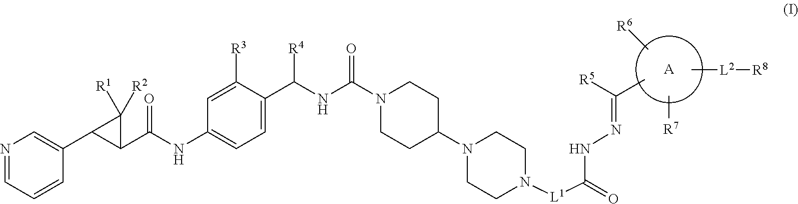
or a pharmaceutically acceptable salt and/or solvate thereof,
wherein:
Ring A is phenyl, a 5 or 6 membered unsaturated heterocycloalkyl or a 5 or 6 membered heteraromatic ring, the latter two groups comprising 1 to 4 heteroatoms selected from O, N, and S, and Ring A is optionally substituted with one or two additional substituents independently selected from CN, NO₂, halo, C₁₋₆alkyl, C₁₋₆fluoroalkyl, ═O, OR⁹ and SR⁹;
R¹ and R² are independently selected from D and H;
R³ is selected from H and halo;
R⁴ is selected from H, C₁₋₄alkyl, and C₁₋₄fluoroalkyl;
R⁵ is selected from H, C₁₋₄alkyl and C₁₋₄fluoroalkyl;
R⁶ is absent or selected from H, CN, NO₂, halo, C₁₋₆alkyl, C₁₋₆fluoroalkyl, OR¹⁰, SR¹⁰ and NR¹⁰R¹¹, and when present R⁶ is adjacent to 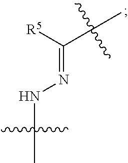
or
R⁵ and R⁶ are joined to form, together with the atoms therebetween, a 4 to 7 membered saturated or unsaturated ring, optionally containing one or two heteroatoms selected from O, N, S, S(O) and S(O)₂ and optionally substituted with one or more substituents independently selected from C₁₋₆alkyl and C₁₋₆fluoroalkyl;
R⁷ is selected from H, halo, C₁₋₆alkyl, C₁₋₆fluoroalkyl, OR¹², SR¹² and NR¹²R¹³;
R⁸ is a reactive functional group;
X is selected from O, S and NR¹⁴;
R⁹, R¹⁰, R¹¹, R¹², R¹³ and R¹⁴, are independently selected from H, C₁₋₆alkyl and C₁₋₆fluoroalkyl; and
L¹ and L² are independently a linker moiety,
provided when Ring A is phenyl, R⁵ and R⁶ are joined to form, together with the atoms therebetween, a 4 to 7 membered saturated or unsaturated ring, optionally containing one one or two heteroatoms selected from O, N, S, S(O) and S(O)₂ and optionally substituted with one or more substituents independently selected from C₁₋₆alkyl and C₁₋₆fluoroalkyl, and Ring A is optionally substituted with one or two additional substituents independently selected from CN, NO₂, halo, C₁₋₆alkyl, C₁₋₆fluoroalkyl, OR⁹ and SR⁹, or when Ring A is phenyl, R⁷ is OH and Ring A is 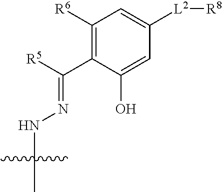
and optionally substituted with one or two additional substituents independently selected from CN, NO₂, halo, C₁₋₆alkyl, C₁₋₆fluoroalkyl, OR⁹ and SR⁹.

2. The compound of claim 1, wherein L¹ and L² independently comprise at least one ester, carbonate, carbamate or amide linkage and optionally one or more ether, sulfone, sulfoxide, thioether, thioamide, thioester and amine, and optionally one or more C₁-C₂₀alkylene groups, C₂-C₂₀alkenylene groups and C₂-C₂₀alkynylene groups.

3. The compound of claim 1, wherein L¹ and L² are independently selected from a direct bond, Z, R^{a}, Z—R^{a}, R^{a}—Z, R^{a}—Z—R^{b} and Z—R^{a}—Z^{a}, wherein Z and Z^{a} are independently selected from O, S, S(O), S02, NH, N(C₁₋₆alkyl), C(Q), C(Q)Y, YC(Q), YC(Q)Y^{a}, (C₁₋₆alkyleneY)ₚ and Y—(C₁₋₆alkyleneY)ₚ, wherein R^{a} and R^{b} are independently selected from C₁₋₁₀alkylene, C₂₋₁₀alkenylene and C₂₋₁₀alkynylene; Q, Y and Y^{a} are independently selected from O, S, NH and N(C₁₋₆alkyl); and p is selected from 1, 2, 3, 4, 5 and 6.

4. The compound of claim 3, wherein R^{a} and R^{b} are independently selected from C₁₋₆alkylene, C₂₋₆alkenylene and C₂₋₆alkynylene.

5. The compound of claim 3 or 4, wherein Q, Y and Y^{a} are independently selected from O, S, NH and N(CH₃).

6. The compound of any one of claims 3 to 5, wherein Z and Z^{a} are independently selected from O, S, S(O), SO₂, NH, N(CH₃), C(O), C(O)NH, NHC(O), NHC(O)O, OC(O)O, NHC(O)NH, OC(O)NH, NHC(NH)NH, (C₁₋₆alkyleneO)ₚ and O—(C₁₋₆alkyleneO)ₚ.

7. The compound of claim 1, wherein L¹ is selected from OC(O)C₁₋₁₀alkyleneO, NHC(O)C₁₋₁₀alkyleneO, C₁₋₆alkyleneO, OC(O)C₁₋₁₀alkyleneNH, NHC(O)C₁₋₁₀alkyleneNH, C₁₋₆alkyleneNH, C(O)C₁₋₁₀alkyleneO and C(O)C₁₋₁₀alkyleneNH.

8. The compound of any one of claims 1 to 7, wherein L² is selected from C₁₋₁₀alkyleneS and C₁₋₁₀alkylene.

9. The compound of any one of claims 1 to 8, wherein R¹ and R² are both D.

10. The compound of any one of claims 1 to 8, wherein R¹ and R² are both H.

11. The compound of any one of claims 1 to 10, wherein the ring to which R¹ and R² are bonded has the following stereochemistry:

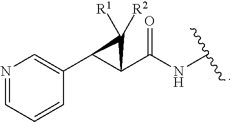

12. The compound of any one of claims 1 to 11, wherein R³ is F.

13. The compound of any one of claims 1 to 12, wherein R⁴ is selected from H, CH₃ and CF₃.

14. The compound of claim 13, wherein R⁴ is H.

15. The compound of any one of claims 1 to 14, wherein X is O.

16. The compound of any one of claims 1 to 15, wherein Ring A is a 5 or 6 membered heteroaromatic ring.

17. The compound of claim 16, wherein Ring A is selected from pyridinyl, pyrimidinyl, pyrazinyl and pyridazinyl.

18. The compound of any one of claims 1 to 17, wherein L¹ is located in the position para to 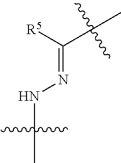
on Ring A.

19. The compound of any one of claims 1 to 18, wherein Ring A is optionally substituted with one or two substituents independently selected from CH₃, CF₃, CH₂CH₃, CH₂CH₂F, CH₂CF₂H and CH₂CF₃

20. The compound of any one of claims 1 to 19, wherein R⁶ is absent.

21. The compound of any one of claims 1 to 19, wherein R⁶ is selected from H, CN, halo, C₁₋₆alkyl and C₁₋₆fluoroalkyl.

22. The compound of any one of claims 1 to 21, wherein R⁵ is selected from H and CH₃.

23. The compound of any one of claims 1 to 19, wherein R⁵ and R⁶ are joined to form, together with the atoms therebetween, a 5 to 6 membered saturated or unsaturated carbocyclic ring, optionally substituted with one or more substituents independently selected from C₁₋₆alkyl and C₁₋₆fluoroalkyl.

24. The compound of any one of claims 1 to 19, wherein R⁵ and R⁶ are joined to form, together with the atoms therebetween, a 4 to 7 membered unsaturated ring, containing one or two heteroatoms selected from O, N, S, S(O) and S(O)₂ and optionally substituted with one or more substituents independently selected from C₁₋₆alkyl and C₁₋₆fluoroalkyl.

25. The compound of any one of claims 1 to 24, wherein R⁷ is selected from H, OH, CH₃, CF₃, CH₂CH₃, CH₂CH₂F, CH₂CF₂H and CH₂CF₃.

26. The compound of any one of claims 1 to 15, wherein, Ring A is a 5 or 6 membered unsaturated heterocycloalkyl ring, and Ring A is optionally substituted with one or two additional substituents independently selected from CH₃, CF₃, CH₂HC₃, CH₂CH₂F, CH₂CF₂H, CH₂CF₃ and ═O.

27. The compound of any one of claims 1 to 15, wherein, Ring A is phenyl and R⁵ and R⁶ are joined to form, together with the atoms therebetween, a 5 to 6 membered unsaturated ring, containing one or two heteroatoms selected from O, N, S, S(O) and S(O)₂, and Ring A is optionally substituted with one or two additional substituents independently selected from CN, NO₂, halo, C₁₋₆alkyl, C₁₋₆fluoroalkyl, OR⁹ and SR⁹.

28. The compound of claim 27, wherein Ring A is phenyl and R⁵ and R⁶ are joined to form, together with the atoms therebetween, a 5 to 6 membered unsaturated ring, containing one heteroatom selected from O, N and S.

29. The compound of claim 28, wherein the heteroatom is N or O.

30. The compound of any one of claims 27 to 29, wherein R⁷ is located in a position ortho to 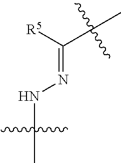
on Ring A, and is selected from H, Cl, F, CH₃, CF₃ and OR¹².

31. The compound of any one of claims 1 to 15, wherein Ring A is phenyl and R⁵ and R⁶ are joined to form, together with the atoms therebetween, a 5 to 6 membered unsaturated carbocyclic ring, and Ring A is optionally substituted with one or two additional substituents independently selected from CN, NO₂, halo, C₁₋₆alkyl, C₁₋₆fluoroalkyl, OR⁹ and SR⁹.

32. The compound of any one of claims 1 to 15, wherein Ring A is 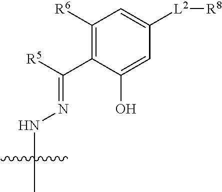
optionally substituted with one or two additional substituents independently selected from CN, NO₂, halo, C₁₋₆alkyl, C₁₋₆fluoroalkyl, OR⁹ and SR⁹.

33. The compound of claim 32, wherein R⁵ is CH₃.

34. The compound of any one of claims 1 to 33, wherein each R⁹, R¹⁰, R¹¹, R¹², R¹³ and R¹⁴ are independently selected from H and C₁₋₄alkyl.

35. The compound of any one of claims 1 to 34, wherein R⁸ is selected from a Michael addition acceptor, an amine, a maleimide, a N-hydroxysuccinimide ester and a thiol.

36. The compound of claim 1, wherein the compound of Formula (I) is selected from: 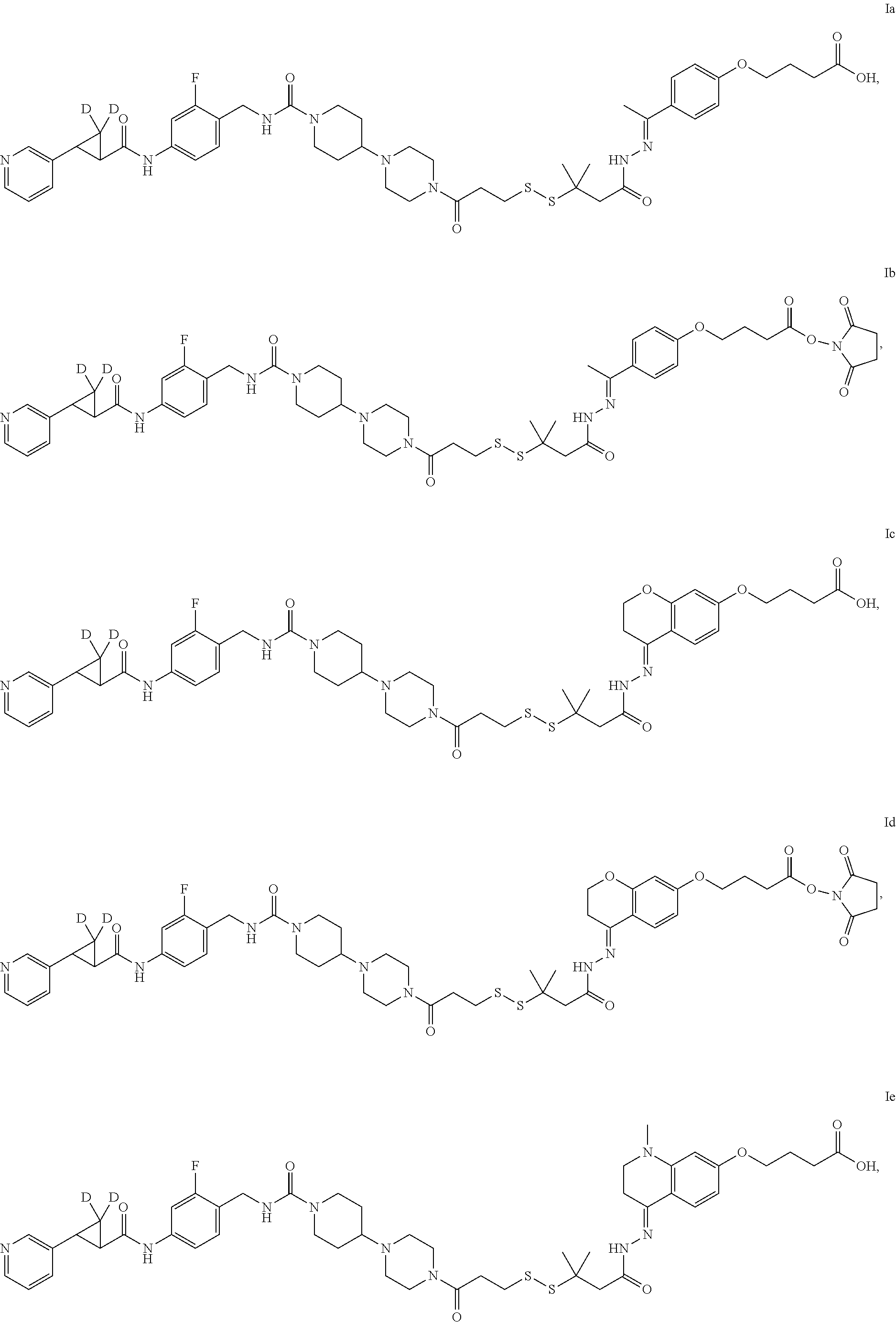 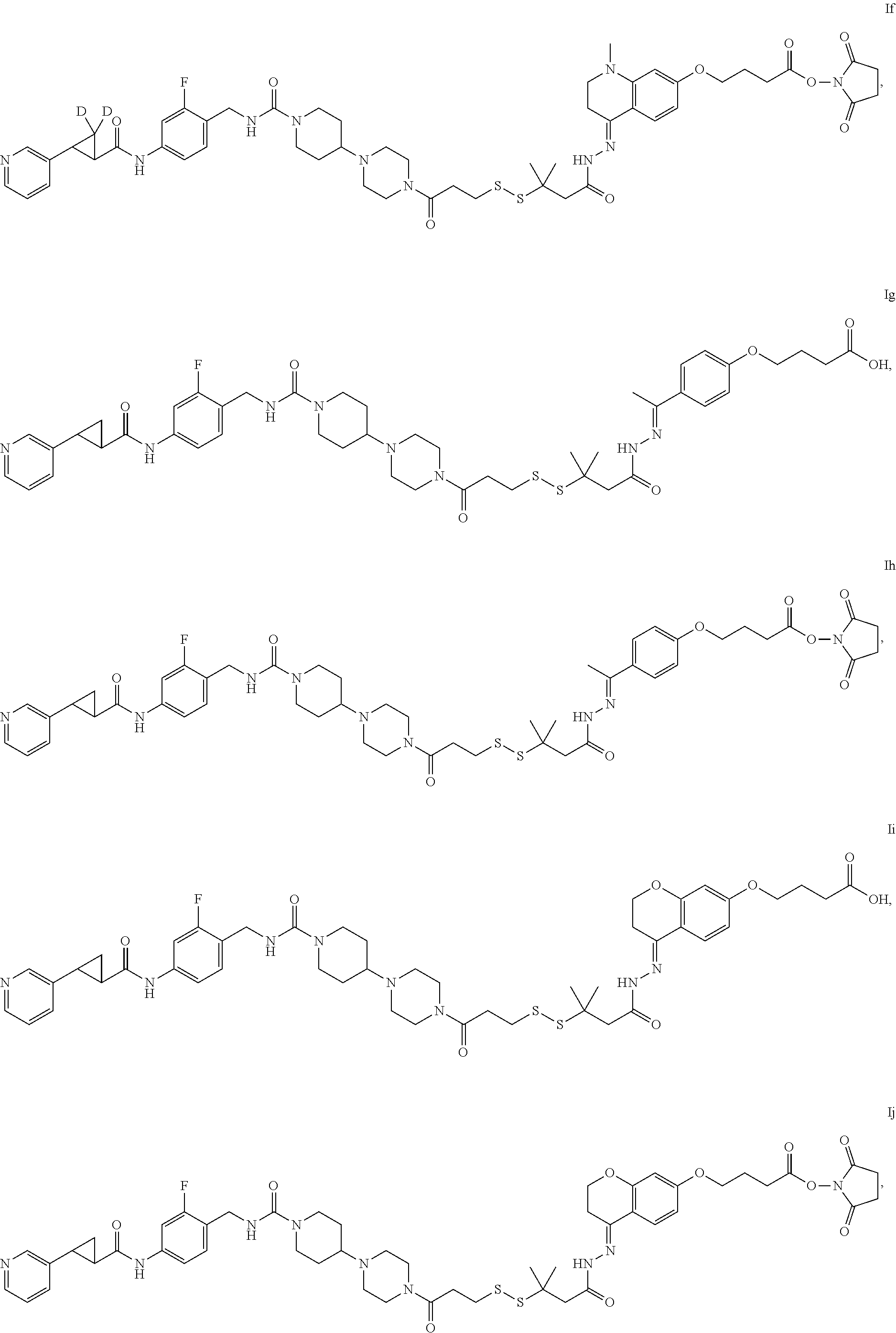 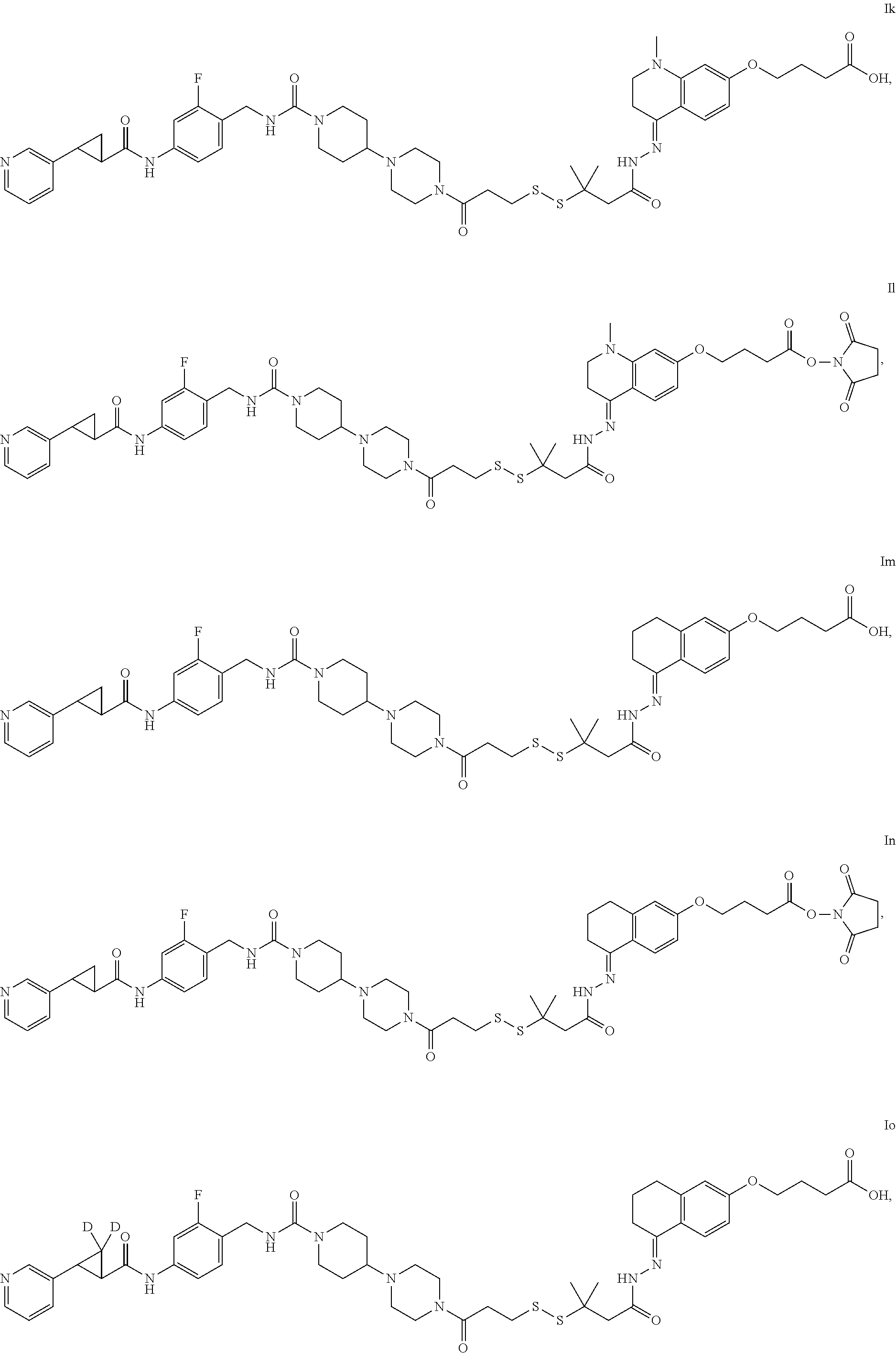 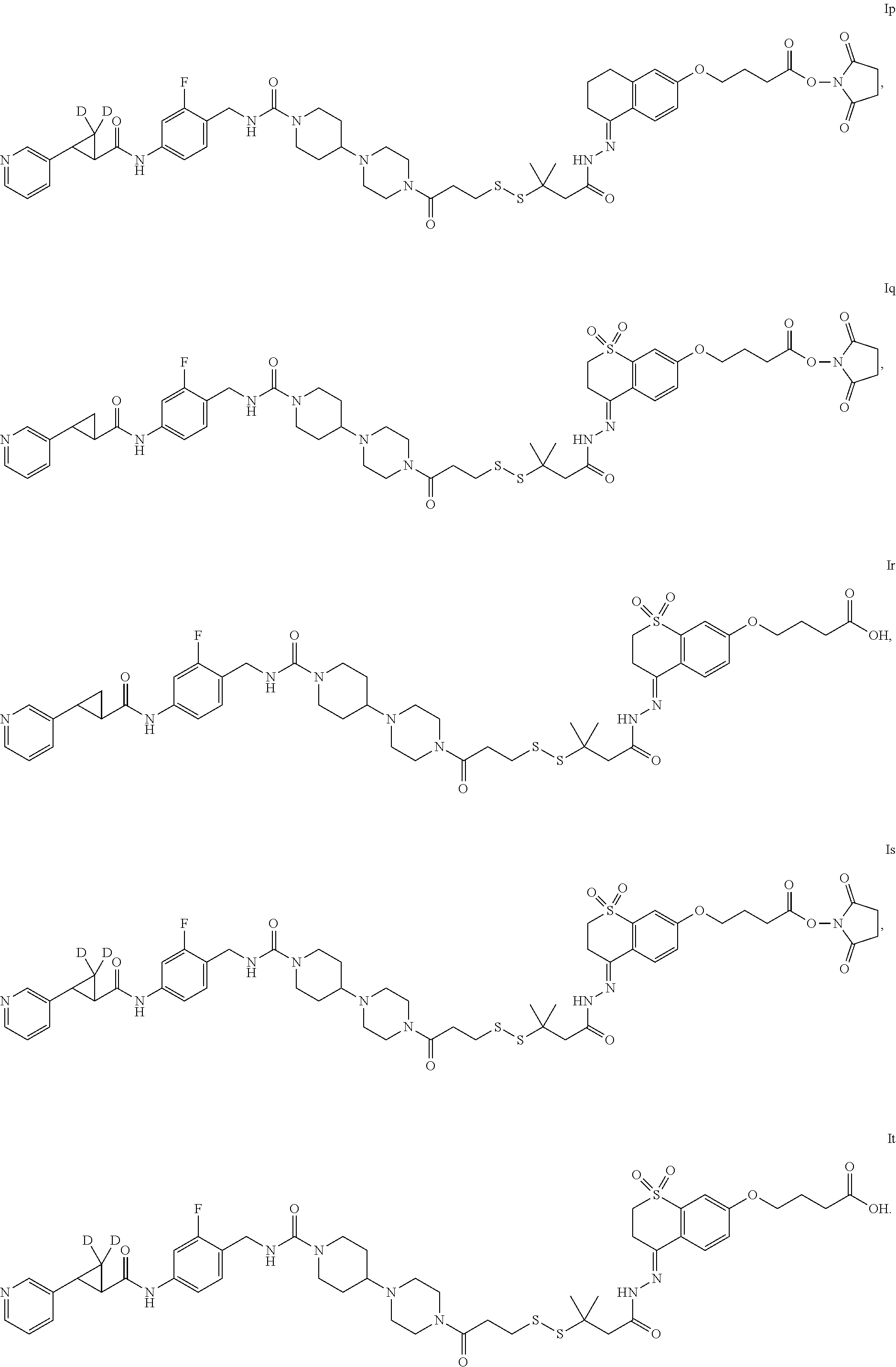 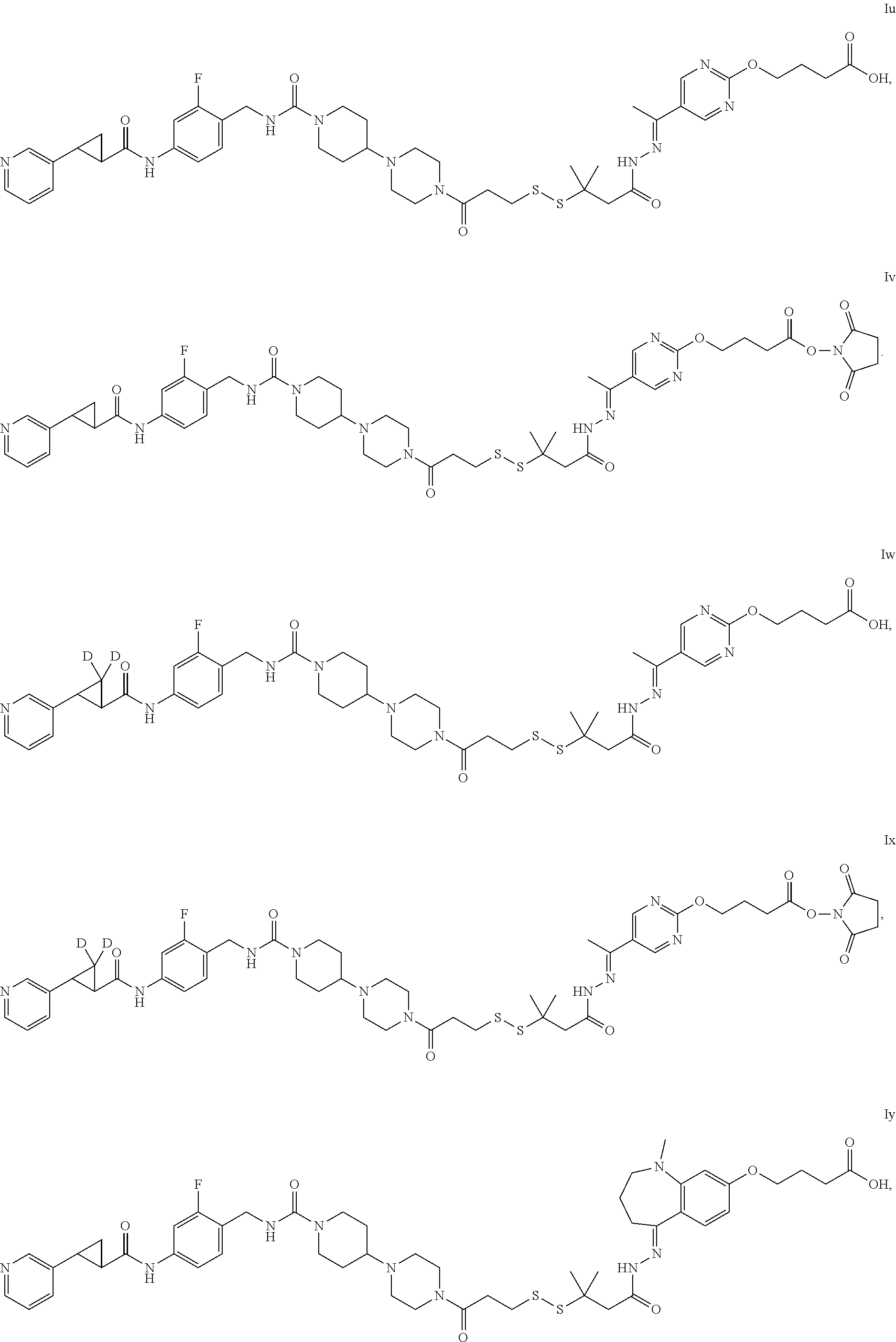 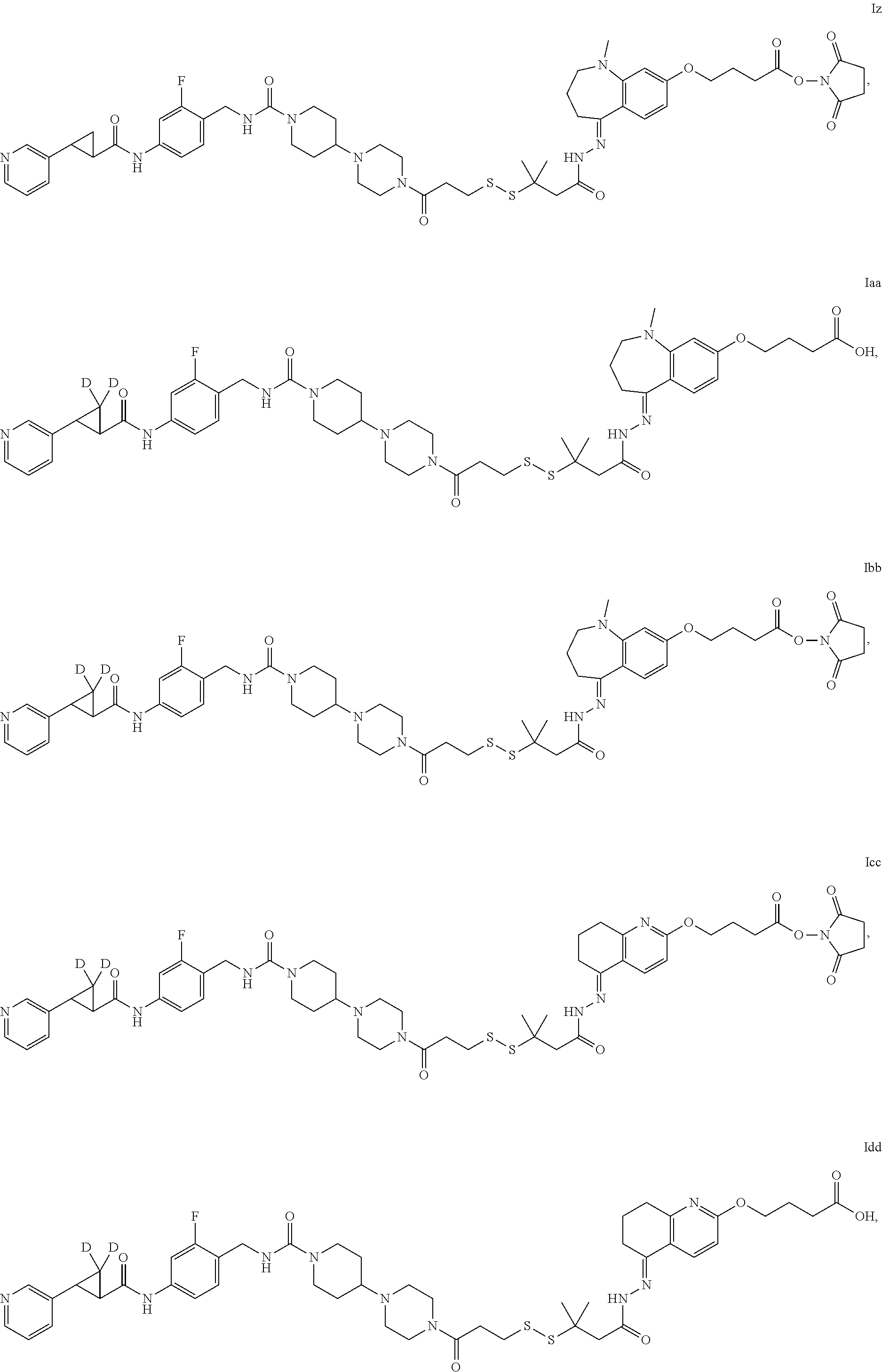 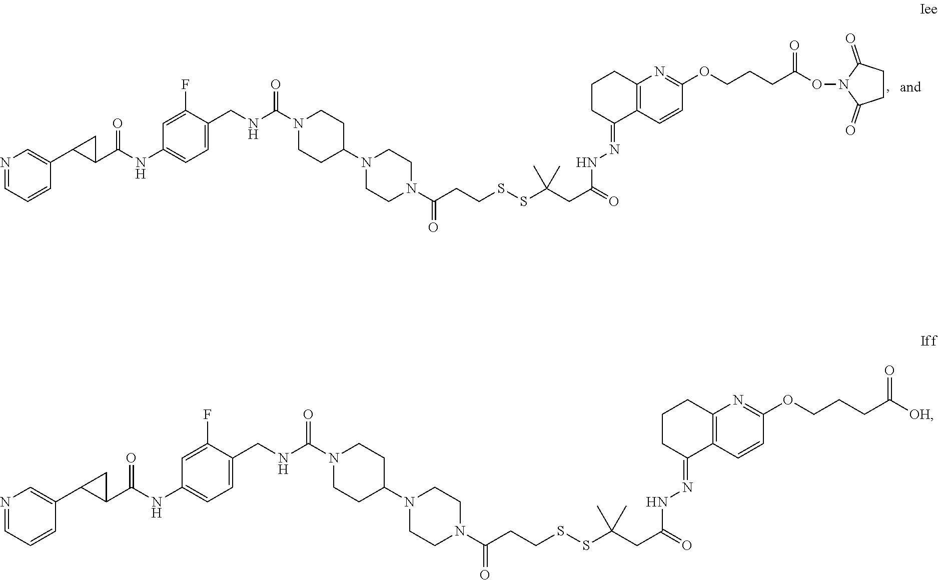 [SMILES: CC(C)(CC(=O)N/N=C1\CCCc2nc(OCCCC(=O)ON3C(=O)CCC3=O)ccc21)SSCCC(=O)N1CCN(C2CCN(C(=O)NCc3ccc(NC(=O)C4CC4c4cccnc4)cc3F)CC2)CC1 ; CC(C)(CC(=O)N/N=C1\CCCc2nc(OCCCC(=O)O)ccc21)SSCCC(=O)N1CCN(C2CCN(C(=O)NCc3ccc(NC(=O)C4CC4c4cccnc4)cc3F)CC2)CC1]
or a pharmaceutically acceptable salt and/or solvate thereof.

37. A compound of Formula (II): 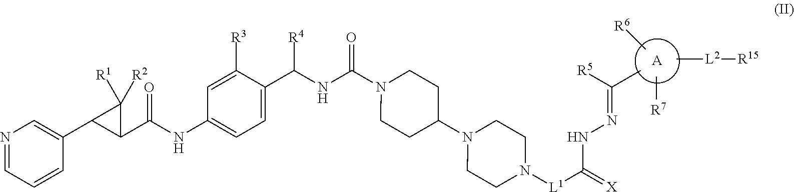
or a pharmaceutically acceptable salt and/or solvate thereof,
wherein
Ring A is phenyl, a 5 or 6 membered unsaturated heterocycloalkyl or a 5 or 6 membered heteraromatic ring, the latter two groups comprising 1 to 4 heteroatoms selected from O, N, and S, and Ring A is optionally substituted with one or two additional substituents independently selected from CN, NO₂, halo, C₁₋₆alkyl, C₁₋₆fluoroalkyl, ═O, OR⁹ and SR⁹;
R¹ and R² are independently selected from D and H;
R³ is selected from H and halo;
R⁴ is selected from H, C₁₋₄alkyl, and C₁₋₄fluoroalkyl;
R⁵ is selected from H, C₁₋₄alkyl and C₁₋₄fluoroalkyl;
R⁶ is absent or selected from H, CN, NO₂, halo, C₁₋₆alkyl, C₁₋₆fluoroalkyl, OR¹⁰, SR¹⁰ and NR¹⁰R¹¹, and when present R⁶ is adjacent to 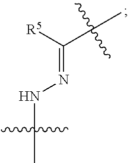
or
R⁵ and R⁶ are joined to form, together with the atoms therebetween, a 4 to 7 membered saturated or unsaturated ring, optionally containing one or two heteroatoms selected from O, N, S, S(O) and S(O)₂ and optionally substituted with one or more substituents independently selected from C₁₋₆alkyl and C₁₋₆fluoroalkyl;
R⁷ is selected from H, halo, C₁₋₆alkyl, C₁₋₆fluoroalkyl, OR¹², SR¹² and NR¹²R¹³;
R¹⁵ is a compound to be linked;
X is selected from O, S and NR¹⁴;
R⁹, R¹⁰, R¹¹, R¹², R¹³ and R¹⁴, are independently selected from H, C₁₋₆alkyl and C₁₋₆fluoroalkyl; and
L¹ and L² are independently a linker moiety,
provided when Ring A is phenyl, R⁵ and R⁶ are joined to form, together with the atoms therebetween, a 4 to 7 membered saturated or unsaturated ring, optionally containing one or two heteroatoms selected from O, N, S, S(O) and S(O)₂ and optionally substituted with one or more substituents independently selected from C₁₋₆alkyl and C₁₋₆fluoroalkyl, and Ring A is optionally substituted with one or two additional substituents independently selected from CN, NO₂, halo, C₁₋₆alkyl, C₁₋₆fluoroalkyl, OR⁹ and SR⁹, or
when Ring A is phenyl, R⁷ is OH and Ring A is 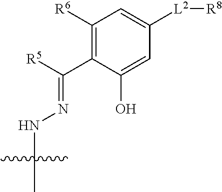
and optionally substituted with one or two additional substituents independently selected from CN, NO₂, halo, C₁₋₆alkyl, C₁₋₆fluoroalkyl, OR⁹ and SR⁹.

38. An antibody-drug conjugate (ADC), the conjugate having a Formula (III) 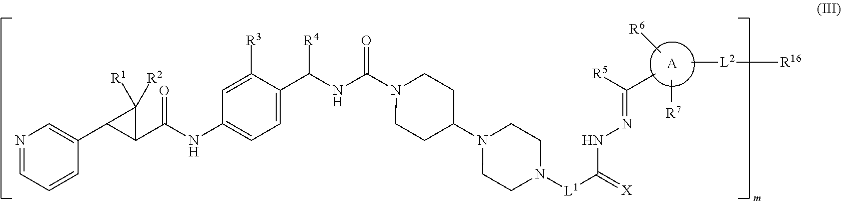
wherein
Ring A is phenyl, a 5 or 6 membered unsaturated heterocycloalkyl or a 5 or 6 membered heteraromatic ring, the latter two groups comprising 1 to 4 heteroatoms selected from O, N, and S, and Ring A is optionally substituted with one or two additional substituents independently selected from CN, NO₂, halo, C₁₋₆alkyl, C₁₋₆fluoroalkyl, ═O, OR⁹ and SR⁹;
R¹ and R² are independently selected from D and H;
R³ is selected from H and halo;
R⁴ is selected from H, C₁₋₄alkyl, and C₁₋₄fluoroalkyl;
R⁵ is selected from H, C₁₋₄alkyl and C₁₋₄fluoroalkyl;
R⁶ is absent or selected from H, CN, NO₂, halo, C₁₋₆alkyl, C₁₋₆fluoroalkyl, OR¹⁰, SR¹⁰ and NR¹⁰R¹¹, and when present R⁶ is adjacent to 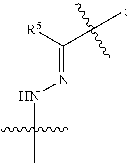
or
R⁵ and R⁶ are joined to form, together with the atoms therebetween, a 4 to 7 membered saturated or unsaturated ring, optionally containing one or two heteroatoms selected from O, N, S, S(O) and S(O)₂ and optionally substituted with one or more substituents independently selected from C₁₋₆alkyl and C₁₋₆fluoroalkyl;
R⁷ is selected from H, halo, C₁₋₆alkyl, C₁₋₆fluoroalkyl, OR¹², SR¹² and NR¹²R¹³;
R¹⁶ is an antibody;
X is selected from O, S and NR¹⁴;
R⁹, R¹⁰, R¹¹, R¹², R¹³ and R¹⁴, are independently selected from H, C₁₋₆alkyl and C₁₋₆fluoroalkyl;
L¹ and L² are independently a linker moiety, and
m is an integer from 1 to 20,
provided when Ring A is phenyl, R⁵ and R⁶ are joined to form, together with the atoms therebetween, a 4 to 7 membered saturated or unsaturated ring, optionally containing one or two heteroatoms selected from O, N, S, S(O) and S(O)₂ and optionally substituted with one or more substituents independently selected from C₁₋₆alkyl and C₁₋₆fluoroalkyl, and Ring A is optionally substituted with one or two additional substituents independently selected from CN, NO₂, halo, C₁₋₆alkyl, C₁₋₆fluoroalkyl, OR⁹ and SR⁹, or
when Ring A is phenyl, R⁷ is OH and Ring A is 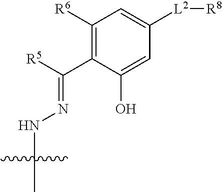
and optionally substituted with one or two additional substituents independently selected from CN, NO₂, halo, C₁₋₆alkyl, C₁₋₆fluoroalkyl, OR⁹ and SR⁹.

39. The antibody-drug conjugate of claim 39, wherein the antibody specifically binds to a receptor encoded by an ErbB gene, a c-Kit gene or a CD30 gene,

40. The antibody-drug conjugate of claim 38 or claim 39, wherein m is an integer from 1-10.

41. A compound of Formula (IV): 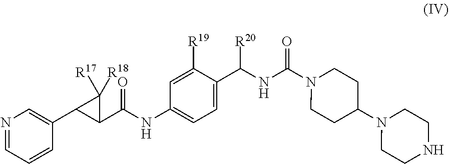
or a pharmaceutically acceptable salt and/or solvate thereof,
wherein:
R¹⁷ and R¹⁸ are independently selected from D and H;
R¹⁹ is selected from H and halo; and
R²⁰ is selected from H, C₁₋₄alkyl, and C₁₋₄fluoroalkyl;
provided at least one of R¹⁷ and R¹⁸ is D.

42. The compound of claim 41, wherein R¹⁷ and R¹⁸ are both D.

43. The compound of claim 41 or claim 42, wherein R¹⁹ is F.

44. The compound of any one of claims 41 to 43, wherein R²⁰ is selected from H, CH₃ and CF₃.

45. A pharmaceutical composition comprising one or more compounds of Formula (II) of claim 37 or a pharmaceutically acceptable salt and/or solvate thereof, and a pharmaceutically acceptable carrier and/or diluent.

46. A pharmaceutical composition comprising one or more compounds of Formula (III) of any one of claims 38 to 40, or a pharmaceutically acceptable salt and/or solvate thereof, and a pharmaceutically acceptable carrier and/or diluent.

47. A pharmaceutical composition comprising one or more compounds of Formula (IV) of any one of claims 41 to 44, or a pharmaceutically acceptable salt and/or solvate thereof, and a pharmaceutically acceptable carrier and/or diluent.

48. A method of inhibiting NAMPT in a cell, either in a biological sample or in a patient, comprising administering an effective amount of one or more compounds of Formula (II) of claim 37 or a pharmaceutically acceptable salt and/or solvate thereof, and/or one or more compounds of Formula (III) of any one of claims 38 to 40 or a pharmaceutically acceptable salt and/or solvate thereof, and/or one or more compounds of Formula (IV) of any one of claims 41 to 44 or a pharmaceutically acceptable salt and/or solvate thereof, to the cell.

49. A method of treating a disease, disorder or condition by inhibition of NAMPT comprising administering a therapeutically effective amount of one or more compounds of Formula (II) of claim 37 or a pharmaceutically acceptable salt and/or solvate thereof, and/or one or more compounds of Formula (III) of any one of claims 38 to 40 or a pharmaceutically acceptable salt and/or solvate thereof, and/or one or more compounds of Formula (IV) of any one of claims 41 to 44 or a pharmaceutically acceptable salt and/or solvate thereof, to a subject in need thereof.

50. A method of treating and/or diagnosing one or more diseases, disorders or conditions comprising administering an effective amount of one or more compounds of Formula (II) of claim 37 or a pharmaceutically acceptable salt and/or solvate thereof, and/or one or more compounds of Formula (III) of any one of claims 38 to 40 or a pharmaceutically acceptable salt and/or solvate thereof, to a subject in need thereof.

51. The method of claim 49 or claim 50, wherein the disease, disorder or condition is a neoplastic disorder.

52. The method of claim 51, wherein the neoplastic disorder is cancer.

53. The method of claim 52, wherein the cancer is selected from breast cancer, skin cancer, prostate cancer, head and neck cancer, colorectal cancer, pancreatic cancer, kidney cancer, lung cancer and brain cancer.

54. The method of claim 52, wherein the cancer is an ErbB-expressing cancer, a c-Kit-expressing cancer or a CD30 expressing cancer.

55. A method of preparing an ADC of Formula (III) as defined in claim 38 comprising:
(a) reacting a compound of Formula (I) as defined in any one of claims 1 to 37 with an antibody to provide the ADC of Formula (III); and optionally
(b) purifying the ADC of Formula (III).

56. A method of preparing a compound of Formula E 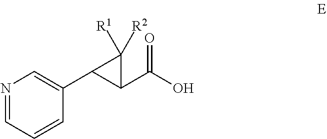
wherein R¹ and R² are both H or R¹ and R² are both D, comprising reacting a compound of Formula D 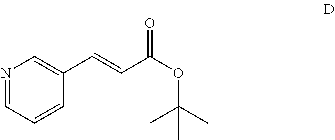
with trimethylsulfoxonium iodide or trimethylsulfoxonium-d₉ iodide.
